SEQUENCE LISTING (45 sequences)

SEQ ID NO: 1 - DNA, 1521 bp, Arabidopsis thaliana
atggacagagtggtggcaaaaatagcaaaaatccgttcgcagcttactaaattacgttcactattcttcctttacttcatctacttcttgttcttctcctttttagggtttttggcactcaagatcacaaagccaagaaccacttcacgtcctcatgactttgaccttttcttcacttctgtctctgccatcaccgtctcttccatgtctaccgtcgacatggaagtcttctccaacacccaacttatcttcctcactatcctcatgttcctcggtggcgaaatcttcacctcctttctcaacctctacgtctcctatttcaccaagttcgtcttccctcataacaagattagacatattttgggatcttataattcggacagttccatcgaggatcgctgtgacgttgagactgttactgattatcgcgagggtcttatcaagatcgatgaaagggcatctaagtgcttgtactcggtggttcttagttaccatcttgttactaacctagttggctctgtgttgcttcttgtgtacgtaaattttgttaaaacggcgagagatgttcttagttccaaagaaatctcacctctcactttctccgtcttcacaactgtttccacgtttgcaaactgcggatttgtccccacgaatgagaacatgatcatctttcgcaagaactctggtctcatctggctcctaatccctcaagtactgatgggaaacactttgttcccttgcttcttggttttgctcatatggggactttataagatcacaaagcgtgacgagtatggttacattctcaagaaccacaataagatgggatactctcatctactctcggttcgtctatgtgttcttcttggagtgacggtgctagggtttctgataatacagcttcttttcttctgcgcctttgaatggacctctgagtctctagaaggaatgagttcgtacgagaagttggttggatcgttgtttcaagtggtgaattcgcgacacaccggagaaactatagtagacctctctacactttccccagctatcttggtactctttattcttatgatgtatcttcctccatacactttatttatgccgttgacggaacaaaagacgatagagaaagaaggaggagatgatgattccgaaaatggaaagaaagttaaaaagagtggactcatcgtgtcacaactttcctttttgacgatatgtatctttctcatttcaatcaccgaaaggcaaaatctacaacgtgatccgataaatttcaacgtccttaacatcactctcgaagttatcagtgcatatggaaacgttggtttcactaccgggtacagctgtgaacggcgtgtggacatcagcgatggtggctgcaaagacgcgagttatgggtttgcaggacgatggagtccaatgggaaaattcgtactaataatagtaatgttttatggtaggtttaagcagttcacagccaaatctggccgcgcatggattctttacccctcgtcttcctaa

SEQ ID NO: 2 - protein, 506 aa, Arabidopsis thaliana
MDRVVAKIAKIRSQLTKLRSLFFLYFIYFLFFSFLGFLALKITKPRTTSRPHDFDLFFTSVSAITVSSMSTVDMEVFSNTQLIFLTILMFLGGEIFTSFLNLYVSYFTKFVFPHNKIRHILGSYNSDSSIEDRCDVETVTDYREGLIKIDERASKCLYSVVLSYHLVTNLVGSVLLLVYVNFVKTARDVLSSKEISPLTFSVFTTVSTFANCGFVPTNENMIIFRKNSGLIWLLIPQVLMGNTLFPCFLVLLIWGLYKITKRDEYGYILKNHNKMGYSHLLSVRLCVLLGVTVLGFLIIQLLFFCAFEWTSESLEGMSSYEKLVGSLFQVVNSRHTGETIVDLSTLSPAILVLFILMMYLPPYTLFMPLTEQKTIEKEGGDDDSENGKKVKKSGLIVSQLSFLTICIFLISITERQNLQRDPINFNVLNITLEVISAYGNVGFTTGYSCERRVDISDGGCKDASYGFAGRWSPMGKFVLIIVMFYGRFKQFTAKSGRAWILYPSSS

SEQ ID NO: 3 - DNA, 3817 bp, Arabidopsis thaliana
gcttaaaccgactcgagaactaaaatggacagagtggtggcaaaaatagcaaaaatccgttcgcagcttactaaattacgttcactattcttcctttacttcatctacttcttgttcttctcctttttagggtttttggcactcaagatcacaaagccaagaaccacttcacgtcctcatgactttgaccttttcttcacttctgtctctgccatcaccgtctcttccatgtctaccgtcgacatggaagtcttctccaacacccaacttatcttcctcactatcctcatgttcctcggtggcgaaatcttcacctcctttctcaacctctacgtctcctatttcaccaagttcgtcttccctcataacaagattagacatattttgggatcttataattcggacagttccatcgaggatcgctgtgacgttgagactgttactgattatcgcgagggtcttatcaagatcgatgaaagggcatctaagtgcttgtactcggtggttcttagttaccatcttgttactaacctagttggctctgtgttgcttcttgtgtacgtaaattttgttaaaacggcgagagatgttcttagttccaaagaaatctcacctctcactttctccgtcttcacaactgtttccacgtttgcaaactgcggatttgtccccacgaatgagaacatgatcatctttcgcaagaactctggtctcatctggctcctaatccctcaagtactgatgggaaacactttgttcccttgcttcttggttttgctcatatggggactttataagatcacaaagcgtgacgagtatggttacattctcaagaaccacaataagatgggatactctcatctactctcggttcgtctatgtgttcttcttggagtgacggtgctagggtttctgataatacagcttcttttcttctgcgcctttgaatggacctctgagtctctagaaggaatgagttcgtacgagaagttggttggatcgttgtttcaagtggtgaattcgcgacacaccggagaaactatagtagacctctctacactttccccagctatcttggtactctttattcttatgatgtaagtttctttcaatcctctcctcatatatcgaatcatttgcatgaactcgaaatatttagatcgaaaggtgcacttatttcgacataattttcacatatgaggtgatatgcatgcacacataggtgcagttttacgaatacattaaactaaaacaattgagatatagtgaaattaaatggttgacatttcactacagaaacaataaaaagtcaccgcatgaattaaagttggttgacatttcacaacagaaacaccaaacatattattatctcacgtaatgtagtttggaaaattatcaaatcatttacctaatttttttttttatgaagcaaacattttcctaactattattgtttattccataccaaaaaaaaaaacgtttatgatttatttaaggatttttttaattactacgtaaaggccaatcatgcatgagtggggtggtagaaccatagatttgcattagttttagtgctattcttttggctgaatccaccaattaatcagcttagcaattgaaagcatattttattaaccaaaattaattttaacatatctaacgtggtccatttggttaaagtattcactcccatggattgaggaagtaagatttaagttataaccatgcttggcaattatacatttaatgaatagtttgaataccaagctaatttataatttttggagattaattagtatttatttcggtaaatcattcttgtaacaaaaaaaatactaataatataaaaaattgagattgtgaagtttattttttcaccacaaacacaaatttcatagaatttttcaaggtttccacaaattagtttacgctcttaaaataacaaacaaaacaaaaagtagtatgcttgtcaaactttccacaaaaacattaacgccggaaaacttaaaacaaatagtcataatacatctttattaaataaataaaaagaaggcataatacatatataagttgataaaactctccatcacttaagagtaattttttgatgttttcacttagaagaatttatatatatctatattaaccattaattaaggaacctacaagagaatgtggtagctagacaagatttgatagagtgaatcagaatatatgtccatggttttagattcagtaaaaaaaaaatgtcccttcaatgtagtggagactggagaatgtcccttcatcatattcacaaagcattattctctgtttttccataaaaacttctttggactaattaaaaagtaaaatataattgcgtttccaaggtatcttcctccatacactttatttatgccgttgacggaacaaaagacgatagagaaagaaggaggagatgatgattccgaaaatggaaagaaagttaaaaagagtggactcatcgtgtcacaactttcctttttgacgatatgtatctttctcatttcaatcaccgaaaggcaaaatctacaacgtgatccgataaatttcaacgtccttaacatcactctcgaagttatcaggtatgtttctatatctcaagattctttaaccaaaaccaaataaaaaaattgtttcagcaatatataaatctctatattacatatcatgcatccataataactaaaaatttcaaaatattttcaaacaaaatacgtcatctaggtgatacattattctacaatttgacaacccaagtattcgtaattcacctaacaaaaaaagaggacgtgaataaaatcgtataagaaacatatatacagtaaaatctctataaattaataatgttgggactgcaaaattttattaatttagagagattttttacttatcgataaattaataaattaatagagagatcttcataatttaataactttgaaaaaatttctcattatagaaaatatctttctaaaatcaattacaagtaaaaaataatatcatgtatagaaaacaacaccaatatgttttgatatagtaaaatattagaactaaatttcaaataagaatatatacaaatttaagcaaaaaaatatcagaaatatatttttaagaaatttcttacatataatgtatatatatagttgatatatttgtgcaattattaatttctgatattgatggaccatatatttacataggactttcaaaaaaattattatctaattaatttatcgatttatgtcattttttatactactcccaactcgggaccggaagaatttattaatttatagaggttattaatttatcgagtataaatttataaaataaaaagaaacatatatatatatataatatatatatatatatataattcactcattatgaattgattgtgaattagaagttaagaattatcaacaaaataagttttgtaatggcagtgcatatggaaacgttggtttcactaccgggtacagctgtgaacggcgtgtggacatcagcgatggtggctgcaaagacgcgagttatgggtttgcaggacgatggagtccaatgggaaaattcgtactaataatagtaatgttttatggtaggtttaagcagttcacagccaaatctggccgcgcatggattctttacccctcgtcttcctaacatatttcgatcatatataaatgttatattaaaattagtcacttttagcattccttttctgtgagtgatgtacaataagtttgtatgtagtttgcatcatcgttaatcctcgttaatattatgataatgtatg

SEQ ID NO: 4 - DNA, 4308 bp, Artificial sequence
  expression cassette
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctcctcctcccatctataaattcctccccccttttcccctctctatataggaggcatccaagccaagaagagggagagcaccaaggacacgcgactagcagaagccgagcgaccgccttcttcgatccatatcttccggtcgagttcttggtcgatctcttccctcctccacctcctcctcacagggtatgtgcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggtacggaatcttgcgattttgtgagtaccttttgtttgaggtaaaatcagagcaccggtgattttgcttggtgtaataaaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttcatttaaatcaactagggatatcacaagtttgtacaaaaaagcaggcttcacaatggacagagtggtggcaaaaatagcaaaaatccgttcgcagcttactaaattacgttcactattcttcctttacttcatctacttcttgttcttctcctttttagggtttttggcactcaagatcacaaagccaagaaccacttcacgtcctcatgactttgaccttttcttcacttctgtctctgccatcaccgtctcttccatgtctaccgtcgacatggaagtcttctccaacacccaacttatcttcctcactatcctcatgttcctcggtggcgaaatcttcacctcctttctcaacctctacgtctcctatttcaccaagttcgtcttccctcataacaagattagacatattttgggatcttataattcggacagttccatcgaggatcgctgtgacgttgagactgttactgattatcgtgagggtcttatcaagatcgatgaaagggcatctaagtgcttgtactcggtggttcttagttaccatcttgttactaacctagttggctctgtgttgcttcttgtgtacgtaaattttgttaaaacggcgagagatgttcttagttccaaagaaatctcacctctcactttctccgtcttcacaacagtttccacgtttgcaaactgcggatttgtccccacgaatgagaacatgatcatctttcgcaaaaactctggtctcatctggctcctaatccctcaagtactgatgggaaacactttgttcccttgcttcttggttttgctcatatggggactttataagatcacaaagcgtgacgagtatggttacattctcaagaaccacaataagatgggatactctcatctactctcggttcgtctatgtgttcttcttggagtgacggtgctagggtttctgataatacagcttcttttcttctgcgcctttgaatggacctctgagtctctagaaggaatgagttcgtacgagaagttggttggatcgttgtttcaagtggtgaattcgcgacacaccggagaaactatagtagacctctctacactttccccagctatcttggtactctttattcttatgatgtatcttcctccatacactttatttatgccgttgacggaacaaaagacgatagagaaagaaggaggagatgatgattccgaaaatggaaagaaagttaaaaagagtggactcatcgtgtcacaactttcctttttgacgatatgtatctttctcatttcaatcaccgaaaggcaaaatctacaacgtgatccgataaatttcaacgtccttaacatcactctcgaagttatcagtgcatatggaaacgtgggtttcactaccgggtacagctgtgaacggcgtctggacatcagcgatggtggctgcaaagacgcgagttatgggtttgcaggacgatggagtccaatgggaaaattcgtactaataatagtaatgttttatggtaggtttaagcagttcacagtcaaatctggccgcgcatggatactttacccctcgtcttcctaacaacccagctttcttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattcattttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaac

SEQ ID NO: 5 - DNA, 52 bp, Artificial sequence
  primer prm3283
ggggacaagtttgtacaaaaaagcaggcttcacaatggacagagtggtggca

SEQ ID NO: 6 - DNA, 52 bp, Artificial sequence
  primer prm3286
ggggaccactttgtacaagaaagctgggtgaaatatgttaggaagacgaggg

SEQ ID NO: 7 - protein, 112 aa, Artificial sequence
  domain
GLIVSQLSFLTICIFLISITERQNLQRDPINFNVLNITLEVISAYGNVGFTTGYSCERRLDISDGGCKDASYGFAGRWSPMGKFVLIIVMFYGRFKQFTAKSGRAWILYPSS

SEQ ID NO: 8 - protein, 15 aa, Artificial sequence
  domain
EVISAYGNVGFTTGY

SEQ ID NO: 9 - protein, 15 aa, Artificial sequence
  consensus sequence
EXXSAXGXXGXXXGY

SEQ ID NO: 10 - protein, 5 aa, Artificial sequence
  signature
DPXNX

SEQ ID NO: 11 - DNA, 1632 bp, Mesembryanthemum crystallinum
atggggagatttgggtttcttaaggagaaagttcagcaactttatggttgtctttgtgtaggcttattttaccttcttagtactttgttttgggtatccaacaaaatttatgactttattacctatcgtatgagtcatttttccattgaagtgtgttacttcatctttgtgtcgtgtttagggttcttaatcttgaggaatataaagcctaggacgtatccggttaaacctggagaattagatatgttctttacctcggtctcggccgcaacagtttcatccatggctactgttgaaatggaggtattttcagatgcccaactccatattatgaccattttgatgttcataggaggggaggttttcacttccatggtaggccttcattttaaggcctctaggcttgggaatacacccttaggtgtaaaaagtagggctaattctgtggctagtttaccatgcccaccagaagattttgatcacattgagttaggcatcatcactaccacaaccaccacaacaacaacaaccacaaccacattgcaaaaaactaaatcagaaattgatttcctcttaaagtctagatcaattagggttttgggttttgtagttttggcctacctactaatagttcatgtcttaggcacaataatggtctatgcataccttagaattgagcctagtgcgaagagggttctagagactaaagggttaaaatctatcacattcgcgattttcactagtgtttcaacattttcaagctgtgggttcgtccctaccaatgaaaacatgatcatttttcgacaaaattccggcctcctcttgatgctaatccctcaagtcctcctaggaaacacattgttaccatctttcttaagattaacaatttgggtcctgggaaaatttacaaagaaggatgaatctaagtatcttatgaggaacacaaaggagattgcataccatcatttgctccctaccaagcactctaagtatgtggttgtgaccgttttcgggttcattttggcgtcactcattatgttttgctcaatggattggaacttgaagggattaagtgatcttaatgtttaccaaaaactggtgggtgcattgttccaatgtgtgaatgcaagacatactggtgaaactattgttgatctctcaactattgcctcagctgtcttggttgttttcgtcataatgatgtatcttcccccttatacatcatttctgccaacaaaagatggagaagaagaatacccactagtatacaaaggagaaaagacaaaggggaagttgattttggacaatgtggtattctcacaactctcctaccttgtcatcttcatcatccttgtttgcatcacagagagaaaaagcatgaaagaagatcccctcaatttcaatgttctcaacattgttgttgaagttattagtgcatatggaaatgttggattttcaactggctacagttgttcaaggcaattaaaaccagatgcaaattgtgtagataaatggtatggatttgtaggaaaatggagtgaccaagggaagatcatcctcatttttgtcatgttctttggaaggctcaagaaattcaacatgaaaggaggcagagcatggaagctcctataa

SEQ ID NO: 12 - protein, 543 aa, Mesembryanthemum crystallinum
MGRFGFLKEKVQQLYGCLCVGLFYLLSTLFWVSNKIYDFITYRMSHFSIEVCYFIFVSCLGFLILRNIKPRTYPVKPGELDMFFTSVSAATVSSMATVEMEVFSDAQLHIMTILMFIGGEVFTSMVGLHFKASRLGNTPLGVKSRANSVASLPCPPEDFDHIELGIITTTTTTTTTTTTTLQKTKSEIDFLLKSRSIRVLGFVVLAYLLIVHVLGTIMVYAYLRIEPSAKRVLETKGLKSITFAIFTSVSTFSSCGFVPTNENMIIFRQNSGLLLMLIPQVLLGNTLLPSFLRLTIWVLGKFTKKDESKYLMRNTKEIAYHHLLPTKHSKYVVVTVFGFILASLIMFCSMDWNLKGLSDLNVYQKLVGALFQCVNARHTGETIVDLSTIASAVLVVFVIMMYLPPYTSFLPTKDGEEEYPLVYKGEKTKGKLILDNVVFSQLSYLVIFIILVCITERKSMKEDPLNFNVLNIVVEVISAYGNVGFSTGYSCSRQLKPDANCVDKWYGFVGKWSDQGKIILIFVMFFGRLKKFNMKGGRAWKLL

SEQ ID NO: 13 - DNA, 1518 bp, Mesembryanthemum crystallinum
atggagaaatatttggctctcttccacaagaaattagatcaattttttaatttttgttatacaaaagctttatcatatattagctccttgtatgagtacataatcttccaagttcacccattttggcatcacctcttttattatatcatagtttctctcttgggttacttctccttgaaggccacttcaagaaagcagaggagctcccctgttttctctgatccccgccacgatgacctcgacctcttcttcacctctgtctccgccaccaccatctcgagcatgtcgacaatcgagatggaggatttctcgagtccccagctcgtcgttctcatcatcctcatgctttctggtggggaagtcttcctttccctccttggcctccagctccgtaaatccaagcataggaagagggagcaaaagaacatcctcttgaactcgccctccgaaggtatgaaatataagtcactggtggcactaggacatgttgttttgggctacctcttggtgtgccatataattgggtacagtctagcttccttgtacataagcatttctagaagtgcaagcaatgtgctcgagagaaaacacattgagatgcatctcttctccctcttcgtcaccgtctccacattctccaactgtggattcatcccaacaaatgagaacatgatggtctttaagggaaattcaggattgctattgatcctcatcccacaaatcctcctagggaacaaactgtacccatcttgcctaaggctagttctttgggtgctcgaaaagctaaccggcaaagccgagttcagctacatattgaagaaaaatgatgagcttggttatgggttgtttttctcgcaagtcgatgcgttgctcttggctgtgactgcggttggattggttatggttcagtttgtggtgttttgcatcttggagtggaattcggtagcgttgatggatcttaattggcatcaaaaactggtcggatctctctttcaaactgtcaattctaggcacagtggagaatctatcattgatctctcccttatttcccctgccaccatggttctcttcgttgtcatgatgtatcttccatcctataccacatttatacctgttggatacgacaaagaaatatcgccagaaatgagcagcaagaagagcaaaaagcaaggttgcagcattgcagaaaatctcaaattctctcagctttcttacttggctatctttgttatgattgtttgcatcactgaaaggagaaatttagtggaagaccctctcaacttcaacgtcttcaacattatcgtagaagtcataagtgcatatggaaatgttggattctccatgggttatagttgtaaaagaagacttggagacattggaacttgcaaggatgcaggatatggcttcgtgggtcgatggagtagacaagggaaaatggttatcatcttggtcatgctttttggtaggcttaagagatttaattttaaaggcggaaaagcttggaaactttctctttaa

SEQ ID NO: 14 - protein, 505 aa, Mesembryanthemum crystallinum
MEKYLALFHKKLDQFFNFCYTKALSYISSLYEYIIFQVHPFWHHLFYYIIVSLLGYFSLKATSRKQRSSPVFSDPRHDDLDLFFTSVSATTISSMSTIEMEDFSSPQLVVLIILMLSGGEVFLSLLGLQLRKSKHRKREQKNILLNSPSEGMKYKSLVALGHVVLGYLLVCHIIGYSLASLYISISRSASNVLERKHIEMHLFSLFVTVSTFSNCGFIPTNENMMVFKGNSGLLLILIPQILLGNKLYPSCLRLVLWVLEKLTGKAEFSYILKKNDELGYGLFFSQVDALLLAVTAVGLVMVQFVVFCILEWNSVALMDLNWHQKLVGSLFQTVNSRHSGESIIDLSLISPATMVLFVVMMYLPSYTTFIPVGYDKEISPEMSSKKSKKQGCSIAENLKFSQLSYLAIFVMIVCITERRNLVEDPLNFNVFNIIVEVISAYGNVGFSMGYSCKRRLGDIGTCKDAGYGFVGRWSRQGKMVIILVMLFGRLKRFNFKGGKAWKLSL

SEQ ID NO: 15 - DNA, 1653 bp, Eucalyptus camaldulensis
atgatgagctttttttcgtctttaggcaaagaggttgcgttcctttgtagcgcttcatggatcaaactagcctgcatctgcaggtccctttgcttcctcctttcttgctgtttccgcttcttgctcctccgagtcaattccttctgcatccaagttttttacttcgtcttcctctcgttcctcggcttctgggtcctcaaggccctagggccgcggaccgattcctttaggcctagggacttggatctgttcttcacttccgtctctgcgacgaccgtctcgagcatgtccacggtcgagatggaagtcttctccaactcacagcttgtcgtgatgaccgtcctgatgttcatcggaggagaggtcttcatttccctcgttgggcttcatcttaggaaatccaagctccggtggcgcatcagaaccgaagacaaagtcgcctctgccgatggcaatctctgcccttccgccccaacaaatgacattgttgaccacatagagttaggtgtagtggcgaaaacagattgcctgaactcgcaggtagagcctcaattttacagaccgcaagacaagtcctcagatcttgactatttgaaatattgttcggttaggtttttgtgttatgtagtgttaggttaccttctggtagtccaagttctaggagttgccgcagtttcgctctatatcacactagttcctagcgccagagatgtgctaaagaagaaaggtctcaaaatggtgaccttttccgtcttcaccaccgtgtcgacctttgccagttgtgggtttgtcccgaccaatgaaaacatgattatcttcagcaaaaactctggcctcctcctgattctcatccctcaggcccttcttgggaacatgctgttcccatcgagcctacgtttgacgctttggctcatcgggagattcagcgagaaagatgaaatcgggtacttgttgagcaggacaagtgaaattggttacaagcacttgcttccgagcctctactcgtcgctactgggggtcacggtgttggggttcgtcggtattcagtttataatgttttgctcgatgcagtgggattcggaatcgttgaatggactaagctcctgtgagaaaatcgtgggcgcgctgtttcagtgcgtgaacagcagacataccggcgagacggtcgtcgatctgtccacagttgctcccgccatcttggtgctattcgtggtcatgatgtaccttccgccttacacgtcgttccttccggtgaaaggcaatgagaggttccctgaaaacggcgaaagaagaaagccgaaacagagttatagactgcttttggagaatctcaaattttcacagctttcctacctggccatctttatcatcgtgatttgcatcaccgagaggaaaaagatgaaggaagatcccctcaatttcaacgtgttgaacatcgtggtcgaagtggtaagcgcatatggaaatgtggggtttacgacaggatacagttgcgaacgccaactaaggcctgtcaaaggctgcgaggacaagtggtatggattctctgggaaatggagcgatgaaagcaaaatcatcctcatcgtagtcatgtttttcggaagattaaagaagttcaacatgaagggcggccgagcttggatactcttgtag

SEQ ID NO: 16 - protein, 550 aa, Eucalyptus camaldulensis
MMSFFSSLGKEVAFLCSASWIKLACICRSLCFLLSCCFRFLLLRVNSFCIQVFYFVFLSFLGFWVLKALGPRTDSFRPRDLDLFFTSVSATTVSSMSTVEMEVFSNSQLVVMTVLMFIGGEVFISLVGLHLRKSKLRWRIRTEDKVASADGNLCPSAPTNDIVDHIELGVVAKTDCLNSQVEPQFYRPQDKSSDLDYLKYCSVRFLCYVVLGYLLVVQVLGVAAVSLYITLVPSARDVLKKKGLKMVTFSVFTTVSTFASCGFVPTNENMIIFSKNSGLLLILIPQALLGNMLFPSSLRLTLWLIGRFSEKDEIGYLLSRTSEIGYKHLLPSLYSSLLGVTVLGFVGIQFIMFCSMQWDSESLNGLSSCEKIVGALFQCVNSRHTGETVVDLSTVAPAILVLFVVMMYLPPYTSFLPVKGNERFPENGERRKPKQSYRLLLENLKFSQLSYLAIFIIVICITERKKMKEDPLNFNVLNIVVEVVSAYGNVGFTTGYSCERQLRPVKGCEDKWYGFSGKWSDESKIILIVVMFFGRLKKFNMKGGRAWILL

SEQ ID NO: 17 - DNA, 1650 bp, Eucalyptus camaldulensis
atgatgcgctttccttgtttaggcaaagaagtcgtgtgtctttgtattgcttcatgcctcaaaatagcccgcctcttcaggtccttctgcttcctcttttcttggtacttccgcttcctgctcctccgagtcaactccttttgtgtccagcttttgtacttcgtcttcctctcgctccttggcttctgggtccttaaggcatcgaggccgctaacccattccttcaggcctagggacttggatctgttcttcacctctgtctccttggctactgtctcgagcatgtccacggtggagatggaggtcctctccgactcccagctcgtcgtgatgaccgtcctgatgttcgtcggaggggaagtcttcacctctcttgccgggcttcacttcaggaaactgaaactccagtcgctcctgaaaaccgaagaaacagtcgcctccgtccataccaatctttgcccttcgaacccaaccaacggtgttgttgaccacgtagagttagctgtagtaacgaattcagattgcctgaactctcgagtagaacctcagttgcacggaccgcgagacgagtccccagatctcgactatttgaaatatcgttccgctaacgttttgtgtcttgtagtgttaggttaccttctgggagtcaatgttctcggtgtcgccgcagtttcactctatatgatacttgtttccagcgcgagagacgtgctaaagcggaagggcctcaaaatgatgacattttccatcttcaccactgtgtcgaccttcgccagttgcgggttcgtcccgacgaacgaaaacatggctgtcttcaacaagaactccggcctcctcctaattctcatccctcaggtccttctcgggaacacgctgttcccgtcgaccttacggctggtactttggctcttgggaaaattcagcaagaaagcagaaatcgattatttgttgagcagaacaggtgaaattggttacaagcacttgcttccgagcctctactcgtcgctcctggtggtaacggtgttggggtttattggtgttcagttcataatgttttgctcaatgcagtgggattcagaatcgttagatggactaaactcgtacgagaaaattgtggccgtgctctttcagtgcgtgaacaccagatatacgggcgagacgatcgttaatctgtccaaagtatctcctgccatcttggtgttattcgtcgtcatgatgtaccttccgccatacacgtccttccttccggcgataggtggtgaggagctcctgggaaatggggaaagaaaaaaggcgaaacgaagtcataaactaatgttgaagagtctcatattctcacagccctcctacttggccatctttatcatcacgatttgcataacggagaaggaaaagatgaagaaagatcctctcaactttaatgtgttcaacatcgtggtcgaagtgataagtgcatatgggaatgtggggttcacgataggttacaattgcgatcggcaactaagacgtattgaaggctgcgaggacaagtggtacggattctctgggaaatggagcgatgagggcaaaattatcctcattgccgtcatgattttcggaagattaaagaagttcaacatgaaaggtggccgaaattggatactcttgtga

SEQ ID NO: 18 - protein, 549 aa, Eucalyptus camaldulensis
MMRFPCLGKEVVCLCIASCLKIARLFRSFCFLFSWYFRFLLLRVNSFCVQLLYFVFLSLLGFWVLKASRPLTHSFRPRDLDLFFTSVSLATVSSMSTVEMEVLSDSQLVVMTVLMFVGGEVFTSLAGLHFRKLKLQSLLKTEETVASVHTNLCPSNPTNGVVDHVELAVVTNSDCLNSRVEPQLHGPRDESPDLDYLKYRSANVLCLVVLGYLLGVNVLGVAAVSLYMILVSSARDVLKRKGLKMMTFSIFTTVSTFASCGFVPTNENMAVFNKNSGLLLILIPQVLLGNTLFPSTLRLVLWLLGKFSKKAEIDYLLSRTGEIGYKHLLPSLYSSLLVVTVLGFIGVQFIMFCSMQWDSESLDGLNSYEKIVAVLFQCVNTRYTGETIVNLSKVSPAILVLFVVMMYLPPYTSFLPAIGGEELLGNGERKKAKRSHKLMLKSLIFSQPSYLAIFIITICITEKEKMKKDPLNFNVFNIVVEVISAYGNVGFTIGYNCDRQLRRIEGCEDKWYGFSGKWSDEGKIILIAVMIFGRLKKFNMKGGRNWILL

SEQ ID NO: 19 - DNA, 1593 bp, Oryza sativa
atgacgagcatttaccatgatttcattcataacaagctgcagagcttcggaaggatcggtagatattttgttaattttgttgttctagcccatagattcattgccttgcatattcacccattctggattcagttgtcctacttccttctcattagcatcctaggttcagttctattgatgttcctgaagccaagcaacccagaattcagaccaggttacattgacatgctgttcttgtcaacctctgctctgacactttccagcctcatcaccatcgaaatggaggttctctcaagctcacaaattgtggttattacactgctgatgcttctcgggggtgaagtctttgtttctttcctgggcctcatgcttagactgaaccataagcacaacccagagttttcaggggacaaggtcagttcagttcctatcgagcttgatacaatcaactcggcaagcactgtgataagttgtgaagagctgcagctggaagcagcaattcctgaagttccatcttccaccattaaggatttgaagaggagcaagcgcctgaggtggttcttaggatttgtagtcttcagctattttgttgtgatccatgtcgctggctttctgctggttctctggtacataagtcgtgtctcatctgcaaaagctccactgaagaagaaagggatcaacattgcactcttctcattctcggtcacggtctcctcgtttgcgaatgtggggctcgtgccgacaaatgagaacatggcaatcttctccaagaacccgggcctcctcctcctgttcatcggccagattcttgcaggcaatacactttaccctctcttcctaaggctattgatatggttcctggggaaggtgaccaagttgagagaactgaagctcatgatcaagaaccctgaggagctacagtatgattatttgcttcctaagttgccaacggcgtttctggcatcaactgtcattggccttatggcttccttggtcacactgttcggtgctgtcgactggaattcttcagtctttgatggactcagctcttaccagaagattatcaatgcattgttcatggcagtgaacgcaaggcactcgggggagaactccatcgactgctcactcatcgcccctgctgttctagtactgttcatcatcttaatgtacttaccaccatcgacaacatttgcactgtccaatggagatgaaaaaactgcaaataagaaagcgaaaagaaaattaggtttagtggtgcagaacttggcattttcacagcttgcctgcatctcagtctttgtgatagttgcattcatcactgagaggagtcgtctcagaaatgatccactcaacttctctgctctgaacatgatatttgaaatcatcagtgcatatgggaatgtagggctatccactggttacagctgttcgaggttgcagaaactgcacccggggagcatctgccaggacaagccgtacagcttgtccggatggtggagtgatgaaggaaagttgctgctagtctttgtgatgctctatggaaggctcaaggccttcacaaaaggtacaggtgaatattggaggctatggtaa

SEQ ID NO: 20 - protein, 530 aa, Oryza sativa
MTSIYHDFIHNKLQSFGRIGRYFVNFVVLAHRFIALHIHPFWIQLSYFLLISILGSVLLMFLKPSNPEFRPGYIDMLFLSTSALTLSSLITIEMEVLSSSQIVVITLLMLLGGEVFVSFLGLMLRLNHKHNPEFSGDKVSSVPIELDTINSASTVISCEELQLEAAIPEVPSSTIKDLKRSKRLRWFLGFVVFSYFVVIHVAGFLLVLWYISRVSSAKAPLKKKGINIALFSFSVTVSSFANVGLVPTNENMAIFSKNPGLLLLFIGQILAGNTLYPLFLRLLIWFLGKVTKLRELKLMIKNPEELQYDYLLPKLPTAFLASTVIGLMASLVTLFGAVDWNSSVFDGLSSYQKIINALFMAVNARHSGENSIDCSLIAPAVLVLFIILMYLPPSTTFALSNGDEKTANKKAKRKLGLVVQNLAFSQLACISVFVIVAFITERSRLRNDPLNFSALNMIFEIISAYGNVGLSTGYSCSRLQKLHPGSICQDKPYSLSGWWSDEGKLLLVFVMLYGRLKAFTKGTGEYWRLW

SEQ ID NO: 21 - DNA, 1593 bp, Oryza sativa
atgacgagcatttaccaagaattcattcataccaagtgtcagagcttcagaagcattggtagatatgttcttcattcaattgtcctcatctatagattcgtttccttgcatgttcacccattctggatccaactgtcctacttcctcctcattagcatcctaggttcagttctattaatgtttctgaagccaagcagcccagagttcaagccaggttacattgacatgttgttcttgtcaacctctgctatgaccgtttccggcctcagcaccatcgaaatggaggttctctcaagctcacaaatcgtggtccttacactgctgatgcttgttgggggtgaagtctttgtttctttccttggcctcatgcttagactgaagcataagcacaatccagaattctcaggggacagggtcagttcagttcctatcgagctcgatacgatcgagccgacaagaactgtgatgagcagtgaagagttgcagatagaagcagcagctccagatgttccatcttccaccattaaggatctgaagaggagcaagcgcctgaggtggttcttaggatttgtggtcttcagctattttgttgtcatccatgtcgttggctttctgctggttctctggtacataagccgtgtctcatctgcaaaggctccactgaagaagaaagggatcaacattgcactcttctcattctcggtcaccgtctcctcgtttgcgaatggggggctcgtgccgacgaatgagaacatggcaatcttctccaagaacccgggcctcctcctcctgttcatcggccagattcttgcaggcaacacactttaccctctcttcctaaggatactgatatggttcctgggaaaggtgaccaagttgaaagacctgaagctcatgatcaagaactctgatgagctacagtatgattatttgcttcctaagttgccgactgcgtttctcgcatcaactgtcattggccttatggcttccttggtcacactgttcggttctgtcgactggaattcttcagtctttgatggactcagctcttaccagaagattatcaatgcattgttcatggcagtgaacgcaaggcactcgggggagaactccatcgactgctcactcatcgcccctgctgttctagtactgttcatcatcttaatgtacttaccaccatcgacaacatttgcactgtccaatggagatgaaaaaactgcaaataagaaagcgaaaagaaaattaggtttagtggtgcggaacctggcattttcacagcttgcctgcaacgcggtctttgtgatagttgcgctcatcactgagaggagtcgtctcagaaatgatccactcaacttctctgctctgaacatgatatttgaagtcatcagtgcatatgggaatgtaggactaaccactggttacagctgttcgaggttgcaaaaactgcacccggggagcatctgccaggacaagccgtacagcttgtccggatggtggagcgatgaaggaaagttgctgctagtctctgtgatgctctatggaaggctcaaggccttcaccaaaggtacgggtgaatattggaggctatggtag

SEQ ID NO: 22 - protein, 530 aa, Oryza sativa
MTSIYQEFIHTKCQSFRSIGRYVLHSIVLIYRFVSLHVHPFWIQLSYFLLISILGSVLLMFLKPSSPEFKPGYIDMLFLSTSAMTVSGLSTIEMEVLSSSQIVVLTLLMLVGGEVFVSFLGLMLRLKHKHNPEFSGDRVSSVPIELDTIEPTRTVMSSEELQIEAAAPDVPSSTIKDLKRSKRLRWFLGFVVFSYFVVIHVVGFLLVLWYISRVSSAKAPLKKKGINIALFSFSVTVSSFANGGLVPTNENMAIFSKNPGLLLLFIGQILAGNTLYPLFLRILIWFLGKVTKLKDLKLMIKNSDELQYDYLLPKLPTAFLASTVIGLMASLVTLFGSVDWNSSVFDGLSSYQKIINALFMAVNARHSGENSIDCSLIAPAVLVLFIILMYLPPSTTFALSNGDEKTANKKAKRKLGLVVRNLAFSQLACNAVFVIVALITERSRLRNDPLNFSALNMIFEVISAYGNVGLTTGYSCSRLQKLHPGSICQDKPYSLSGWWSDEGKLLLVSVMLYGRLKAFTKGTGEYWRLW

SEQ ID NO: 23 - DNA, 1530 bp, Oryza sativa
atgcctattcggctgcatatctttgtcaattctgcaaggcatgccatcaactcatcggcattcatttgtcggttcatcgcataccatcttagcccgcttttgattcacttatcctattttcttatcattgatatacttggttttgtttccttggtggtgctcaggccaagcaaccacaagtacaatcctcgctatgttgatatgtttttcctatcgacatctgcagttacagtcataggattagccaccatacaaatggaggatctttccagctctcagatagctatcctgactctcttgatgttcttagacagtaagatgttcctttcctttcttggccttgtccttgagtcaagcaagcaaaacaagcatgatcctgaaaaccgtagagtcagttcagttactgtgtgcaagcaatcacagctagaagaggcgactccccaaactccatccatgaactccattgatatcaagaagagatgcctcaaatacttagtgtttgtggtgctggcatacatgatcattattcttgtcactggttctctattggtgttcatgtacatagctcatgtttcaagtgctagagatgtgctaacaaggaaaagcatcaacaaagctctcttctcgatatcggtcacagtctcctcattcacaaatggagggttattgccgacaaatgagagtatggttgtattctcctcaaacaatggcctcctgttgctactcattggccagattcttgcaggcagcacactgttccctgtgtttctgaggttggtgatatgggcattgagaggactaagattagcaaaagctgaagagcctgacttcatgatgaacaacagcagcgcagtaggtttcagtcacctgctgcctaacttgcagacaatatttcttgcagttgtggaggttgcttttgtagccatgacagtcatcctcttctgctgcttgaactgggattctgtagtgtttgcagggctcagctccctccagaagataaccaatgcattgttcatggcagtgaatgcaaggcaggcaggagaaaattccattgattgttcccttgttgcgccagcggctttagtactattcatggtcatgatgtacactccatccttgacaaagttgttctcagcatgtcaagatcacaaacgaatcggcccagaaagcgacgatagaacaagcaaggggaaaccattcctgaagatgatggcattttcaccactcggcttcaacaccacagtgataatgctggtctgcatcactgaaaggagatcactctccactgaccctctcaatttatccacatttaacatcatctttgaggtgataagtgcctacgggaacattgggttgtccactggctacagctgctcgaggcagctgcagcaccaggaggggattgcctgccatgagaaggcgtacaacttctcggggtggtggagtgaaccagggaaactgattcttgttcttgcaatgctctgcgggaggctcaactcaaaggattccacaagcgcacgaactaggtga

SEQ ID NO: 24 - protein, 509 aa, Oryza sativa
MPIRLHIFVNSARHAINSSAFICRFIAYHLSPLLIHLSYFLIIDILGFVSLVVLRPSNHKYNPRYVDMFFLSTSAVTVIGLATIQMEDLSSSQIAILTLLMFLDSKMFLSFLGLVLESSKQNKHDPENRRVSSVTVCKQSQLEEATPQTPSMNSIDIKKRCLKYLVFVVLAYMIIILVTGSLLVFMYIAHVSSARDVLTRKSINKALFSISVTVSSFTNGGLLPTNESMVVFSSNNGLLLLLIGQILAGSTLFPVFLRLVIWALRGLRLAKAEEPDFMMNNSSAVGFSHLLPNLQTIFLAVVEVAFVAMTVILFCCLNWDSVVFAGLSSLQKITNALFMAVNARQAGENSIDCSLVAPAALVLFMVMMYTPSLTKLFSACQDHKRIGPESDDRTSKGKPFLKMMAFSPLGFNTTVIMLVCITERRSLSTDPLNLSTFNIIFEVISAYGNIGLSTGYSCSRQLQHQEGIACHEKAYNFSGWWSEPGKLILVLAMLCGRLNSKDSTSARTR

SEQ ID NO: 25 - DNA, 1659 bp, Oryza sativa
atgcatccaccaagtttagtgctagataccttgaagcgtatcaaactatacatagccatgaagctcctgttaccgaattcggaggtgcctcggatctattgggagaaagctcagcatctctgtgggttcctgtccatgaagctcatttccagagccagatgtgtggcaagttctgtcaaacaatcttatagttttctggtttgcaaaagtaaccctttggtagttcagctcgtttactttgtgataatctcatttgctggtttccttgctctgaagaatctcaagccccagggtaagccaggtccaaaggatttggacctattgttcacctctgtgtctacacttactgtctcgagcatggcaacagtagagatggaagacttatctgacaggcaactctgggttctgatccttctgatgctaatgggaggagaggtgttcacatcaatgctagggctctacttcaacaatgccaatgccaacagaaatgagaacagccagagaagtttaccttcaatcagcttggacattgaattcaacagtcctgcaaacaatggggatcacaaaattacggaatgtggccaatcagaagaaactatgtcgcaaaaccaggtacagcaaaacaaaagcataacatataatccttgcgctgtgttggttcgcatagtgacaggttatttcgtagctactgtcatttccagttctgtcatcattattatttacttttggattgattcagatgcaagaaatgtactgaaaagtaaggagatcaatatgtataccttttgcatcttcacagcagtgtcctcgttcgcaaactgtggcttcacgccactaaatagtaacatgcaacccttcagaaagaactgggtccttttgctcctagtgatcccgcagattctagcaggcaacaccttgttttcaccactcttgcggctatgcgtatgggttttggggaaggtcagtggaaaagcagagtatgcttacatccttcagcatcctggggagactggctacaaacatttgcatgtgaggagaaattctgtttacattgttctgagtgttactggactgattctactgcaagtaatgtttatttgctcgtttgagtggaactcagagagcttagagggaatgaattggttacagaaattggtaggattattattccagagtgttaatacaagacaagccggtgaatctatccttgatatatcaacactttctccatcaactctgttgctatttgcagtcgtcatgtaccttccttccgatgcttcatttctcactgccaatgctgataaccagcccttgacagataaaaagacaaactcgattagcagagcactgtggaggaattttaccgttaacaagctttcttgtttagcaatgttcacattcctggcatgcataacagaaaggaagtcaatttcttctgatccactaaatttcaacatcttcagcatagtttttgagataatcagcgctttcggcaatgtaggctactcactaggatacagttgccagaagttgttgaagcctgatgccacttgcaaagatgcttcctacgggttcgtcggcaggtggaccgaggagggtaagctgatagttatcctggtgatgttcctggggaggctcaaggagttcatcctgaaatga

SEQ ID NO: 26 - protein, 552 aa, Oryza sativa
MHPPSLVLDTLKRIKLYIAMKLLLPNSEVPRIYWEKAQHLCGFLSMKLISRARCVASSVKQSYSFLVCKSNPLVVQLVYFVIISFAGFLALKNLKPQGKPGPKDLDLLFTSVSTLTVSSMATVEMEDLSDRQLWVLILLMLMGGEVFTSMLGLYFNNANANRNENSQRSLPSISLDIEFNSPANNGDHKITECGQSEETMSQNQVQQNKSITYNPCAVLVRIVTGYFVATVISSSVIIIIYFWIDSDARNVLKSKEINMYTFCIFTAVSSFANCGFTPLNSNMQPFRKNWVLLLLVIPQILAGNTLFSPLLRLCVWVLGKVSGKAEYAYILQHPGETGYKHLHVRRNSVYIVLSVTGLILLQVMFICSFEWNSESLEGMNWLQKLVGLLFQSVNTRQAGESILDISTLSPSTLLLFAVVMYLPSDASFLTANADNQPLTDKKTNSISRALWRNFTVNKLSCLAMFTFLACITERKSISSDPLNFNIFSIVFEIISAFGNVGYSLGYSCQKLLKPDATCKDASYGFVGRWTEEGKLIVILVMFLGRLKEFILK

SEQ ID NO: 27 - DNA, 1596 bp, Oryza sativa
atgaatcattgtcttgtagtatcccacaaaaaactccaaactttccgcacatttgcagctagcaagttctcttcttttaccaaatctgcacagaagtctataaaatactccttccagttcatctaccaaaacaatccactctttgtccatgtagcttactttgccctgatctcctttgctggatatggatctctaaaggtcctcaagccacgagacaagtcaaatactctgaaagacttggacgtgctatttacttccgtatctgcatcaactgtttcaagcatggctactgttgaaatggaggatttctcaagcgctcaactctgggttttgactattttaatgctgattggtggtgaggtattcacttcaatgcttggcattcactttatgagagccgaatttggtacaaaagagtcagtcagcacaagggatcactcaccttgcattgatattgagtctattacttccacaaaatttggtcccagcacccagggcacaaaagttacagtttcattttctgaactccgcatggaaaatggaggacatgtagagcccaagacgattaaatttttaggttttgtagtgatgggatatcttctaataacaaacttaggcggctccctacttatttacctctaccttaacctggtaccaagtgcacataaaattctaaagagaaaaggcattgggatcatcgtattctcagtatttacagccatctcctcagttggaaattgtggcttcactccagtaaatgagaatatgattatctttcagaagaactccattcttctattgctaattcttcctcagatactagcaggaaatacattatttgcaccatgcttgagattaatggtgtggtcacttgagaagattaccggaaaaaaggattgtcgttacattcttgaatatccaaaggccattggatataaacatcttatgagtaccagggaaagtgtttatttgactttaacagttgtgagcttgatcattctgcaaaccgtattgttcctctctttggagtggagctcggtagctttggatggaatgagcaactatcaaaagatagtatccgctctatttcagtcggtcaatgctaggcatgcaggtgaatctgttacagatctgtcaaacctctcttcagcaatcctagtcctatacaccatcatgatgtatctccctggttacacttcgtttttacccagacatgatggtgaggattctaagaccgagaagataaacaaaagaaaagggctattggagaactggatcttctcacatatgtcttatttggctatctttgtaatgctaatttgcatcacagaacgggactcgatggctacagatccacttaatttcaatgttttcagcatattgtttgaagtcgtcagtgcatatggaaatgtggggttctcggttggctacagctgcaagaggctactgaaccatgatgcacgctgcaaggatgcctcgtacgggtttgcggggaaatggagcgacaatgggaaagcgatcctgatcatcgtcatgcttttcgggaggcttaaaacgtttaacatgaagggtggaagagcctggaagcttagataa

SEQ ID NO: 28 - protein, 531 aa, Oryza sativa
MNHCLVVSHKKLQTFRTFAASKFSSFTKSAQKSIKYSFQFIYQNNPLFVHVAYFALISFAGYGSLKVLKPRDKSNTLKDLDVLFTSVSASTVSSMATVEMEDFSSAQLWVLTILMLIGGEVFTSMLGIHFMRAEFGTKESVSTRDHSPCIDIESITSTKFGPSTQGTKVTVSFSELRMENGGHVEPKTIKFLGFVVMGYLLITNLGGSLLIYLYLNLVPSAHKILKRKGIGIIVFSVFTAISSVGNCGFTPVNENMIIFQKNSILLLLILPQILAGNTLFAPCLRLMVWSLEKITGKKDCRYILEYPKAIGYKHLMSTRESVYLTLTVVSLIILQTVLFLSLEWSSVALDGMSNYQKIVSALFQSVNARHAGESVTDLSNLSSAILVLYTIMMYLPGYTSFLPRHDGEDSKTEKINKRKGLLENWIFSHMSYLAIFVMLICITERDSMATDPLNFNVFSILFEVVSAYGNVGFSVGYSCKRLLNHDARCKDASYGFAGKWSDNGKAILIIVMLFGRLKTFNMKGGRAWKLR

SEQ ID NO: 29 - DNA, 1503 bp, Oryza sativa
atgcccacgtcgcggcgcgcgctcgccggcggcgctttgtccatgcacgtcgcctacttcctcgccatctcctgcctcggctacggcctcctcggggtgctcaaggtgcgggagcccggcgccgcgccgcgccgcatcgaccgcttcttcaccgccgtgtcggccgccacggtgtcgagcatgtccaccgtcgagatggaggtgttctccaacggccagctcgtggtgctcaccgtcctgatgctcctcggcggcgaggtgttcgtctcgctcgtggggctcgcgtccaagtggtccaagctgcggagcgacgccatggacagatcccggcgcgtcgagagccacggcgacgtcgcgctcgccgacatcgacggcggcgacgtcgagaacccgacgtcgtcgggagaggaggcggcgagccgacgccgcccgatggacgcggacacgctgcggcacaatgcggtgcgcgcgctgttctacatcgtgctcgccatcttcgcggtggtgcacgtcgtcggcgccgtggccgtcgcggcgtacgtgctcgcgtcgccgggcgcgaggcggacgctgggggacaagtcgctgaacacgtggacgttcgcggtgttcacgacggtgtcgacgttctccaactgcgggttcatgccgacgaacgagaacatggtggtgttcaagcgggacgcgccgctgcagctgctgctggtgccgcaggtgctggcggggaacacgctgttcgcgccgctgctggcggcgtgcgtgtgggcggcggcggcggcgacgcggcgggaggagctcgtggagatggcgagggaggggggcagggcggcggcggccgggtacgcgcacctgatgccggcgcggcggtgctggatgctggcggcgacggtggcggcgttcgtcgccgtgctgatggcgctggtgtgcggcatggagtggggcggggcgctgcaggggatgagcccgtgggagaaggtggtgaacgcgctgttcctcgccgtgaacgcccggcacaccggcgagtccaccgtcgacctctccatcctcgcgccggccatcctcgtgctcttcgtcctcatgatgtatctacctccgtacacgacgtggttcccatttgaagagaattccactactaaggatagtaatgcagagaaccagggaatcagactgctcgagagtacacttttgtcacaactctcctacctgaccatctttgtcattgccatctgcatcaccgagagaagaaagctcaaagaagaccccctcaacttcagtgtgctaagcattgttgtcgaagttgtcagtgcatatggaaatgtggggttctcaatgggctacagttgcagtagacagatcaatccagaccatctctgcacagataaatggactggtttcgtagggagatggagcgactctggcaaactgatactcatttttgtgatgttctttgggaggctcaagaagttcagcatgaaaggaggcaaagcctggaaacttagttag

SEQ ID NO: 30 - protein, 500 aa, Oryza sativa
MPTSRRALAGGALSMHVAYFLAISCLGYGLLGVLKVREPGAAPRRIDRFFTAVSAATVSSMSTVEMEVFSNGQLVVLTVLMLLGGEVFVSLVGLASKWSKLRSDAMDRSRRVESHGDVALADIDGGDVENPTSSGEEAASRRRPMDADTLRHNAVRALFYIVLAIFAVVHVVGAVAVAAYVLASPGARRTLGDKSLNTWTFAVFTTVSTFSNCGFMPTNENMVVFKRDAPLQLLLVPQVLAGNTLFAPLLAACVWAAAAATRREELVEMAREGGRAAAAGYAHLMPARRCWMLAATVAAFVAVLMALVCGMEWGGALQGMSPWEKVVNALFLAVNARHTGESTVDLSILAPAILVLFVLMMYLPPYTTWFPFEENSTTKDSNAENQGIRLLESTLLSQLSYLTIFVIAICITERRKLKEDPLNFSVLSIVVEVVSAYGNVGFSMGYSCSRQINPDHLCTDKWTGFVGRWSDSGKLILIFVMFFGRLKKFSMKGGKAWKLS

SEQ ID NO: 31 - DNA, 1665 bp, Oryza sativa
atgagttctctggatgccactactcctagatatgacgagtttaaaaggatctaccaccttttccttttccatgcacacccattctggctccaactgctgtacttcctcttcatctccctcttgggtttcttgatgctgaaagctctgccgatgaagaccagcatggtgccgaggcccatggacttggacctgatcttcacgtcggtgtcggcgacgacggtgtcgagcatggtcgccgtcgagatggagtccttctccaactcccagctcctcctcatcaccctcctcatgctgcttggtggtgaggtcttcaccagcatccttggcctctacttcaccaacgccaagtactcctccaagatgatagcaaccttacctgatgatgacgaccatggtggcagtggcaaaccaccaccaccaacgacgtcaccttcgtctaccctagtggagctcgagctcgctcctcccatggacgtcgtcgtcgtcaaccctaccaccactgcgacgacgcacgacgaggtagagctagggttaggacgtcggaacaagcgcggctgcacctgcactactactcacacgtcgtcgtcatcatcggcatcgaagacgacgacgacgaggctactgatgttcgtggtgatggggtaccacgcggtggtgcacgtcgccgggtacacggccatcgtcgtgtacctcagcgccgtcggcggcgcgggggcggtggtcgccgggaaggggatcagcgcgcacacgttcgccatcttcaccgtcgtctcgacgttcgccaactgcgggttcgtgccgacgaacgaagggatggtgtcgttcaggtcgttcccggggctcctcctcctcgtcatgccgcacgtcctcctcgggaacacgctcttcccggtcttcctcaggctggccatcgccgcgctcgagagggtcaccgggtggccggagctcggcgagctcctgatccggcggcggaggggcggcggcgagggctaccaccagctgttgccgagctcgcgcacgcggttcctggccctcaccgtggccgtgctcgtggtggcgcagctggcgctcttctgcgccatggagtggggctccgacgggctgcgggggctcaccgcgggccagaagctcgtcggcgcgctcttcatggcggtcaactcgaggcactccggtgagatggtgctcgacctctccaccgtgtcgtcggccgtcgtcgtgctctacgtggtgatgatgtacctgccaccttacaccacttttgtacctgtccaagacaaacaccagcaaacgggagcacagtccgggcaggagggcagcagcagcagcagcatatggcagaagctgctcatgtcgccgctctcgtgcctagccatcttcatcgtcgtcatctgcatcacggagcggcggcaaatcgccgacgaccccatcaactacagcgtcctcaacatcgtcgtcgaggttatcagtgcgtatggcaatgtggggttcagcacggggtacagctgcgcgaggcaggtgaggcccgacggcagctgcagagacctgtgggttggcttctcagggaagtggagcaaacaagggaagctcactctcatggccgtcatgttctacggcaggctcaagaagttcagcctgcacggtggtcaggcatggaagatagaataa

SEQ ID NO: 32 - protein, 554 aa, Oryza sativa
MSSLDATTPRYDEFKRIYHLFLFHAHPFWLQLLYFLFISLLGFLMLKALPMKTSMVPRPMDLDLIFTSVSATTVSSMVAVEMESFSNSQLLLITLLMLLGGEVFTSILGLYFTNAKYSSKMIATLPDDDDHGGSGKPPPPTTSPSSTLVELELAPPMDVVVVNPTTTATTHDEVELGLGRRNKRGCTCTTTHTSSSSSASKTTTTRLLMFVVMGYHAVVHVAGYTAIVVYLSAVGGAGAVVAGKGISAHTFAIFTVVSTFANCGFVPTNEGMVSFRSFPGLLLLVMPHVLLGNTLFPVFLRLAIAALERVTGWPELGELLIRRRRGGGEGYHHLLPSSRTRFLALTVAVLVVAQLALFCAMEWGSDGLRGLTAGQKLVGALFMAVNSRHSGEMVLDLSTVSSAVVVLYVVMMYLPPYTTFVPVQDKHQQTGAQSGQEGSSSSSIWQKLLMSPLSCLAIFIVVICITERRQIADDPINYSVLNIVVEVISAYGNVGFSTGYSCARQVRPDGSCRDLWVGFSGKWSKQGKLTLMAVMFYGRLKKFSLHGGQAWKIE

SEQ ID NO: 33 - DNA, 1530 bp, Oryza sativa
atgcctattcggctgcatatctttgtcagttctgcaaggcatgccatcaactcgtcggcattgatttgtcggttcatcgcattccatcttagcccgcttttgattcacctgtcctattttcttatcattgatgtacttggttttgttgccttggtggtgctcaggccaagcaaccacaagtacaatcctcgctatatcgatatgtttttcctatcgacatctgcagttacagtcacaggattagccaccacacaaatggaggatctttctagctctcagatagctgtcctgactctcttgatgttcttaggaagtgagatgttcctttcctttcttggccttgtccttgagtcgagcaagcaaaacaagcatgatcctgaaaaccgtagagtaagttcagttaccgtgtgtgagcagtcacatctagaagaggcaattccacaaactccatccatgaactccactgatatcaagaggagctgccacaaatacttagtgtttgtggtgttggcatacatgattattattcttgtcactggttctctattggtgttcatgtacatagctcatgtttcaagtgctagagatgtgctaacaaggaaaagcatcaacaaagctctcttctcgatatcggtcacagtctcctcatttacaaatggagggttattgccgacaaatgagagtatggctgtattctcctcaaacaatggcctcctgttgctactcatcggccagattcttgcaggcagcacactgctccctatgtttctgaggttggtgatatgggcattgagaggactaagattagcaaaagctgaagagccagacttcatgatgaacaacagcagctcagtaggtttcagtcacctgctacctaacttgcagacaatatttcttgcagctgtggaggttgcttttgtaggcatgacagtcatcctcttctgctgcttgaactgggattctgcagtgtttgcagggctaacctccctccagaagataaccaatgcattgttcatggcagtgagtgcaaggcaggcaggagaaaattccattgattgttcccttgttgcgccagcggctttagtactattcatggtcatgatgtacactccatccttgacaaagttgttctcagcatgtcaagatcacaaacaaatcggcccggaaagcgacgatagaacaagcaaggggaaaccattcctgaagacgatggcattttcaccactagccttcaacaccacagtgataatgctggtctgcatcactgaaaggagatcgatctccactgaccctctcaatttctccacatttaacatcatctttgaggtgataagtgcctacgggaacattgggttgtccactggctacagctgctcgagacagctacagcaccaggatgggattgcttgccatgagaagccatacagcttctcggggtggtggagtgaaccagggaagctgattcttgttcttgcgatgctctacgggaggctcaactcaaaggattccacaagcgcacgaactaggtga

SEQ ID NO: 34 - protein, 509 aa, Oryza sativa
MPIRLHIFVSSARHAINSSALICRFIAFHLSPLLIHLSYFLIIDVLGFVALVVLRPSNHKYNPRYIDMFFLSTSAVTVTGLATTQMEDLSSSQIAVLTLLMFLGSEMFLSFLGLVLESSKQNKHDPENRRVSSVTVCEQSHLEEAIPQTPSMNSTDIKRSCHKYLVFVVLAYMIIILVTGSLLVFMYIAHVSSARDVLTRKSINKALFSISVTVSSFTNGGLLPTNESMAVFSSNNGLLLLLIGQILAGSTLLPMFLRLVIWALRGLRLAKAEEPDFMMNNSSSVGFSHLLPNLQTIFLAAVEVAFVGMTVILFCCLNWDSAVFAGLTSLQKITNALFMAVSARQAGENSIDCSLVAPAALVLFMVMMYTPSLTKLFSACQDHKQIGPESDDRTSKGKPFLKTMAFSPLAFNTTVIMLVCITERRSISTDPLNFSTFNIIFEVISAYGNIGLSTGYSCSRQLQHQDGIACHEKPYSFSGWWSEPGKLILVLAMLYGRLNSKDSTSARTR

SEQ ID NO: 35 - DNA, 1602 bp, Triticum aestivum
atgggccgggtgaaaagattttaccaggatttcatccatatcaagctgcatagcttctgccgtatcagtggatatgttgtcgattcaatagcttttgtctatagatttgttgcattgcatgttcaccccttctggatccaactgtcctacttccttgccattgctatacttggttcagtcctcttgatgtcgctgaaaccaagcaaccctgacttcagccctccttacattgacatgttattcttgtcaacttctgctctaacagtttctggcctcagcaccatcacgatggaggatctctcaagctctcaaattgtggttttgacattgctcatgcttataggaggggagatctttgtttcactcttagggctcatgcttagagtgaaccatcaagacatgcaagatcttccaagcgtgaagatcagctcggttcctgtcgagcttgaagagctagacttgcccaacagcatggcactatgtgatgagtcgcagcttgaagaagcagctcatgcaattccacccaagaaatgtacagagttgaagaggagtaggtctgtcaagtgcttaggatatgtggtctttgggtactttgccatgatccatgtcttgggctttctgctggtttttctgtatataactcatgtgccaactgcaagtgccccactgaacaagaaagggatcaacatcgtgctcttctcactatcagtcaccgttgcctcctgtgcgaatgcaggactcgtgcccacaaatgagaacatggtcatcttctcaaagaattcaggcctcttgttgctgctgagtggccagatgctcgcaggcaatacattgttccctctcttcctgaggctactggtgtggttcctggggaggatcacaaaggtgaaggagctgaggctcatgatcaataaccccgaggaagtgcgttttgctaatttgcttgctaggttgccaactgtgtttctctcctcaacggtcgttggccttgtagcagctggggtcacgatgttctgcgctgttgattggaattcttcagtctttgatgggctcagctcttatcagaagactgtcaatgcattcttcatggtggtgaatgcgaggcactcaggggagaattccatcgactgctcgctcatgtcccctgccattatagtactattcatcgtcatgatgtatttgccatcatcagcaacatttgcaccacccagtggagatactaaaaccaccaatgagaacacgaaagggaaagtcaagagagggtcgttggtgcagaatttggcattctcaccgctcgggtgtaacatcatctttgtgatggttgcctgcatcacggaaaggagaaggctcagaaacgatccactcaacttctccaccttgaacatgatatttgaggtcatcagcgcatatggcaatgcagggttatccactggttacagttgttctagactgcatcagctgcacccagagatcatctgccaggacaaaccatacagcttttctggatggtggagtgacggaggaaagtttgtgctaatattggtcatgctctatggaaggcttaaggctttcacactggccacgggtaaatcctggaaagtatga

SEQ ID NO: 36 - protein, 533 aa, Triticum aestivum
MGRVKRFYQDFIHIKLHSFCRISGYVVDSIAFVYRFVALHVHPFWIQLSYFLAIAILGSVLLMSLKPSNPDFSPPYIDMLFLSTSALTVSGLSTITMEDLSSSQIVVLTLLMLIGGEIFVSLLGLMLRVNHQDMQDLPSVKISSVPVELEELDLPNSMALCDESQLEEAAHAIPPKKCTELKRSRSVKCLGYVVFGYFAMIHVLGFLLVFLYITHVPTASAPLNKKGINIVLFSLSVTVASCANAGLVPTNENMVIFSKNSGLLLLLSGQMLAGNTLFPLFLRLLVWFLGRITKVKELRLMINNPEEVRFANLLARLPTVFLSSTVVGLVAAGVTMFCAVDWNSSVFDGLSSYQKTVNAFFMVVNARHSGENSIDCSLMSPAIIVLFIVMMYLPSSATFAPPSGDTKTTNENTKGKVKRGSLVQNLAFSPLGCNIIFVMVACITERRRLRNDPLNFSTLNMIFEVISAYGNAGLSTGYSCSRLHQLHPEIICQDKPYSFSGWWSDGGKFVLILVMLYGRLKAFTLATGKSWKV

SEQ ID NO: 37 - DNA, 1458 bp, Suaeda maritima subsp. salsa
atgttgagtttcaaatttatagtagaaaaatgcaaacatttttatacttctctttatctacttcttgtctatgtttttacatccttatattggttaatttcaaaaatctatgattttattatggtttatgtttgccacttgattattgagttatgttacttcattcttgtatcttcttttggattcttgtttttaaaaaccctaattccaaaatcatctaataatagtaataacaataataattcaataaatggtttagatttgtttttcacctcggtttccgccacaaccgtctcaagcatgtcaaccctagagatggaggtattctcaaattcccaactcattgttttaaccattttgatgttcataggaggtgaggtctttacctccatggtaggtctccatttttcggcctcgaaacttgtgtacactcccctgcaagcaaggagtagagtaaactcagtggctagcttaccacttcctcctgaaagtattgagttaggaatcattattccatcatcaacaacaacaacacaagaaattagggtttcatcaaccattgaaaaaacaaaatcagaaatagatttcctaataaaatcaaaatcaattagggttttaggctttgtggttttgtcttacttaatcatagttcatttcctaggaatttcaatggtattaacatacattaatacaatcccaaatgctaaaaatgttttagacaaaaaaggtcttaaaacattcactttttcaattttttcagttgtttcaacttttgcaagttgtggtttcacccctacaaatgaaaacatgcaagtttttagcaaaaactctggcctcttgttgatgctaatccctcaaatcctccttgggaacacattgtttccttcgtttcttcgattttcgatatggatgctaggaaaatttgtcaaaaaagacgaaaccaagtatctaatgagaaactcaaaggaaattgagtaccatcatttgctctctagcaaacactctaggtttttaattgtgacggttttagggttcattttggtgcaatttataatgttttgttcaatggaatggaatttcgatggtctgaataatgatcataatatttatcaaaaattagtgggaatattatttcaatgtgttaattcaagacatacaggtgaaagcattgttgatctttcctccattaattcagctatgttggtgattttcatcgttatgatgtaccttccaccatatacttcctttctgccaataaaggatgaagaaaaagaatatccaaacatggtattatttcacaaaggagaaaagaaacggaagaagattttgaagaatttcctattttcacagcttggccatattgccatttcatcattattatttgcatcacagagaagcaaaagattaaagatgatcccctcaatttcaacgttttcaacattgcctttgaggttctaa

SEQ ID NO: 38 - protein, 485 aa, Suaeda maritima subsp. salsa
MLSFKFIVEKCKHFYTSLYLLLVYVFTSLYWLISKIYDFIMVYVCHLIIELCYFILVSSFGFLFLKTLIPKSSNNSNNNNNSINGLDLFFTSVSATTVSSMSTLEMEVFSNSQLIVLTILMFIGGEVFTSMVGLHFSASKLVYTPLQARSRVNSVASLPLPPESIELGIIIPSSTTTTQEIRVSSTIEKTKSEIDFLIKSKSIRVLGFVVLSYLIIVHFLGISMVLTYINTIPNAKNVLDKKGLKTFTFSIFSVVSTFASCGFTPTNENMQVFSKNSGLLLMLIPQILLGNTLFPSFLRFSIWMLGKFVKKDETKYLMRNSKEIEYHHLLSSKHSRFLIVTVLGFILVQFIMFCSMEWNFDGLNNDHNIYQKLVGILFQCVNSRHTGESIVDLSSINSAMLVIFIVMMYLPPYTSFLPIKDEEKEYPNMVLFHKGEKKRKKILKNFLFSQLGHIAISSLLFASQRSKRLKMIPSISTFSTLPLRF

SEQ ID NO: 39 - DNA, 3708 bp, Saccharomyces cerevisiae
atgcattttagaagaacgatgagtagagtgcccacattggcatctcttgaaatacgatataaaaaatctttcggccataaatttcgtgattttattgctctatgtggtcactattttgctccagttaaaaaatatatcttccccagttttatcgcggttcactacttctacacgatatccctgacattaataacttcaatcctgctatatcccattaagaataccagatacattgatacattgtttttagcagcgggcgcagttacacaaggtggcttaaatactgtggatatcaacaatctaagcttataccaacaaattgttctgtatatcgtatgctgcatatcaacaccaattgcagttcatagttgcttggcatttgtacggctttactggtttgagcgctacttcgatggtattagagactcttctagacgaaattttaagatgagaagaacgaaaacaatcttagaaagggaactaacagcaagaaccatgaccaagaatagaacaggtacccaaagaacgtcttatcctaggaaacaagctaaaacagatgatttccaagaaaaattgttcagcggagaaatggttaatagagatgagcaggactcagttcacagcgaccagaattctcatgacattagtagggacagcagcaataataatacgaatcacaatggtagcagtggcagtttagatgatttcgttaaggaagacgaaacggatgacaatggagaatatcaggagaacaactcctactcgacggtaggtagttcgtctaacacagttgcagacgaaagtttaaatcagaagcccaagccaagcagtcttcggtttgatgagccacacagcaaacaaagacccgcaagagttccctcagagaaatttgcaaaaagaaggggttcaagagatattagcccagccgatatgtatcgatccattatgatgctacaaggtaagcatgaagcaactgctgaagatgaaggtccccctttagtcatcgggtcccctgcggatggcacaagatataaaagtaatgtcaataagctaaagaaggccaccggcataaatggtaacaaaatcaagattcgagataagggaaatgaaagtaacactgatcaaaattccgtgtcaagtgaagcaaacagtacggcgagcgtttcggacgaaagctcgttacacacaaattttggtaacaaagtaccttcattaagaacaaatactcatagatcaaattcgggcccgatagccattactgataacgcagaaacagacaaaaagcatgggccatcaattcaattcgatataactaaacctcctagaaaaatttcaaaaagagtttcaaccttcgatgatttgaacccaaaatcttccgttctttatcgaaaaaaagcatcgaagaagtacctcatgaaacattttcctaaagcgcggcgaatacggcaacaaattaagagaaggctttctactggttcaattgagaaaaacagcagtaacaatgtttcagatagaaaacctattactgatatggatgatgatgatgatgacgatgacaacgacggcgataacaacgaagaatactttgctgacaacgaaagcggcgatgaagatgaacgagtacagcagtctgaaccacattctgattcagaactcaaatcgcaccaacaacagcaagaaaaacaccaactgcagcagaacctgcaccgcatgtataaaaccaaatcatttgatgataatcgttcaagagcagttcctatggaacgttccaggaccatcgatatggcagaggctaaggatctaaatgagctcgcaaggacgcctgattttcaaaaaatggtctatcaaaattggaaagcccatcatagaaaaaaaccgaactttaggaagaggggatggaataacaagatatttgaacatggtccctatgcatctgacagcgatcgcaattatcctgataatagtaatactggaaacagtattcttcattacgcagagtctattttacatcatgatggctctcataaaaatggaagcgaagaagcctcttccgactctaatgagaatatctattccacgaatggaggaagcgaccacaatggtcttaacaactatcctacttacaacgacgatgaagaaggctattatggtttacatttcgataccgattatgacctagatcctcgtcatgatttatctaaaggcagtggtaaaacgtatctatcatggcaaccaactattggacgtaactcaaacttccttggattaacaagagcccagaaagatgaattaggtggtgtcgagtacagagcaatcaaacttttatgcaccatattggttgtctactacgttggatggcatattgttgcttttgttatgttagtaccttggattattttgaaaaagcattatagtgaagttgttagagatgatggtgtttcacctacatggtggggattttggacagcaatgagtgcatttaatgatttaggtttgacattaactccaaattcaatgatgtcgtttaacaaagctgtatacccattgatcgttatgatttggtttatcattatcggaaatacagggtttcccatccttcttagatgcatcatttggataatgtttaaaatttctcctgatttatcacagatgagagaaagtttaggttttctcttagaccatccacgtcgttgtttcaccttgctatttcctaaggcagctacatggtggctacttttaacgcttgcaggattgaatataactgattggattttatttattattctagattttggctcaacagttgtgaaatcattatcgaaaggctatagagtccttgtcggcctgtttcaatctgttagcacaagaactgctggattcagcgttgtcgatttaagtcaactgcatccttctatccaagtctcctatatgctaatgatgtatgtctccgtattaccattggccatctctattcgacggacaaatgtttacgaggagcaatctttaggactatatggagatatggggggagaaccagaagatacggatactgaagacgatggtaacgatgaagatgacgacgaggaaaacgagagtcacgaaggtcaaagtagtcaaagaagtagttcgaacaacaacaacaataacaacaggaaaaagaaaaagaaaaagaaaactgaaaatccaaatgaaatatctacaaaatcctttatcggtgcccatttaaggaaacagctttcatttgacttgtggtttctatttttagggttatttatcatttgcatttgtgaaggggacaagataaaggacgtacaagaaccaaactttaatatatttgcaattctttttgaaattgttagcgcttacggtacagttgggctatcgctaggttatccggacaccaaccaatcgttttcaagacagtttactacattatctaagttggtgatcatagctatgctgatcagaggcaagaatagaggtctaccatactcactggatcgtgcaattatcttgcctagtgatagacttgaacatattgaccaccttgagggcatgaaattgaagagacaggctagaaccaatacagaagacccaatgacggaacatttcaagagaagtttcactgatgtgaaacatcgttggggagctcttaagcgtaagaccacacattcccgaaatcctaaaaggagcagcacaacgctctaa

SEQ ID NO: 40 - protein, 1235 aa, Saccharomyces cerevisiae
MHFRRTMSRVPTLASLEIRYKKSFGHKFRDFIALCGHYFAPVKKYIFPSFIAVHYFYTISLTLITSILLYPIKNTRYIDTLFLAAGAVTQGGLNTVDINNLSLYQQIVLYIVCCISTPIAVHSCLAFVRLYWFERYFDGIRDSSRRNFKMRRTKTILERELTARTMTKNRTGTQRTSYPRKQAKTDDFQEKLFSGEMVNRDEQDSVHSDQNSHDISRDSSNNNTNHNGSSGSLDDFVKEDETDDNGEYQENNSYSTVGSSSNTVADESLNQKPKPSSLRFDEPHSKQRPARVPSEKFAKRRGSRDISPADMYRSIMMLQGKHEATAEDEGPPLVIGSPADGTRYKSNVNKLKKATGINGNKIKIRDKGNESNTDQNSVSSEANSTASVSDESSLHTNFGNKVPSLRTNTHRSNSGPIAITDNAETDKKHGPSIQFDITKPPRKISKRVSTFDDLNPKSSVLYRKKASKKYLMKHFPKARRIRQQIKRRLSTGSIEKNSSNNVSDRKPITDMDDDDDDDDNDGDNNEEYFADNESGDEDERVQQSEPHSDSELKSHQQQQEKHQLQQNLHRMYKTKSFDDNRSRAVPMERSRTIDMAEAKDLNELARTPDFQKMVYQNWKAHHRKKPNFRKRGWNNKIFEHGPYASDSDRNYPDNSNTGNSILHYAESILHHDGSHKNGSEEASSDSNENIYSTNGGSDHNGLNNYPTYNDDEEGYYGLHFDTDYDLDPRHDLSKGSGKTYLSWQPTIGRNSNFLGLTRAQKDELGGVEYRAIKLLCTILVVYYVGWHIVAFVMLVPWIILKKHYSEVVRDDGVSPTWWGFWTAMSAFNDLGLTLTPNSMMSFNKAVYPLIVMIWFIIIGNTGFPILLRCIIWIMFKISPDLSQMRESLGFLLDHPRRCFTLLFPKAATWWLLLTLAGLNITDWILFIILDFGSTVVKSLSKGYRVLVGLFQSVSTRTAGFSVVDLSQLHPSIQVSYMLMMYVSVLPLAISIRRTNVYEEQSLGLYGDMGGEPEDTDTEDDGNDEDDDEENESHEGQSSQRSSSNNNNNNNRKKKKKKKTENPNEISTKSFIGAHLRKQLSFDLWFLFLGLFIICICEGDKIKDVQEPNFNIFAILFEIVSAYGTVGLSLGYPDTNQSFSRQFTTLSKLVIIAMLIRGKNRGLPYSLDRAIILPSDRLEHIDHLEGMKLKRQARTNTEDPMTEHFKRSFTDVKHRWGALKRKTTHSRNPKRSSTTL

SEQ ID NO: 41 - DNA, 2670 bp, Saccharomyces cerevisiae
atgccaacagctaagaggacgtcatccagggcttcgttggcactgcccttccagttacggttggtgcacaagaaatcatggggccatcggctaagagacttcatttccgggttcttaaaatcatgcagacccattgctaaatacgttttccccaacttcatcgtggtgcactatatctacctgatcacgctgtcgattatcgggtccattctgttatatccgtgcaagaacacggcgttcatcgatgtgctatttctggctgctggagcgtctacacagggcgggctggccaccaagagcactaacgatttcaacctgtaccagcagatagtggtgtacgtcattacattgctgtccacgcctatacttattcatgggtttttggcctttgtcaggctgtattggtttgaaaggtacttcgacaacattagggatatctccaaacagaattttaaactaagaaggaccatgacgttgcaacaaagggaactatcgggcagcagtggcaatgccgctcgaagtaggagtttcaaggacaacctgttccgtgggaagtttgtttccagagaagacccacgacaatccgcttcagatgtgccgatggactctcctgacacgtccgcattgtcctcaatctcaccgttgaatgtttcctcctctaaggaggaatccagtgacacgcaaagctcgcctccaaacttctcaagtaagcgccaaccctcagacgttgacccaagagacatttacaaatcgataatgatgctacaaaaacaacaagagaagagcaacgcaaactccacggattctttttcgagcgagaccaatggacccgctttcattgtgcaggaacgtcatgagagaagagccccccactgctcactgaaacgccattctgtcctgccatcttctcaggaattgaacaagctagcccagacgaaaagtttccagaaattgcttggcttgcggagagatgaaggtgaccatgactactttgacggtgctcctcacaaatatatggtcaccaagaagaaaaaaatatctagaacgcaatcatgtaacatcccaacgtatactgcttcaccgagtcctaaaacctcaggccaagtagttgaaaatcatagaaacttggccaagtcggcgccttcatcttttgttgatgaggagatgagcttttcaccgcaagagtctttgaatttacagttccaagcgcacccgcccaaaccaaaacgacgtgaaggtgatataggccaccccttcaccagaacaatgagcaccaactatctatcgtggcagccaacctttggcagaaactccgtcttcattggactcacaaagcaacaaaaggaggaactcggcggtgtcgaatatcgtgctttgagattgctgtgctgcattctcatggtatactacatcggattcaacattttggcgtttgtgaccatcgttccatgggcctgtacgaggcaccactactcagagattattagacgaaatggagtttctccaacctggtgggggtttttcactgcaatgagtgcattcagcaacttgggtctgtctttgaccgctgattcaatggtttcctttgatactgcgccgtatccgctgattttcatgatgttcttcatcatcataggcaatacaggcttcccaattatgttacgatttatcatttggatcatgttcaagacctcgagagacctatctcagtttaaggaaagtcttgggtttctcttggatcatccgcgcaggtgttttacgttgctgttccccagcggccccacatggtggctgtttacaactttagtcgtcttaaacgctacggattggattcttttcataattctggatttcaactccgctgtagtaaggcaggttgctaaaggttatcgagctctcatgggcctcttccagtctgtatgcacaagaactgctggattcaacgttgttgacttaagtaaattacacccgtccattcaggtgtcttatatgctaatgatgtacgtttcggtcctgccgctggcgatttccattagaagaacgaatgtttatgaggagcaatcgttgggactatacgatagtggacaagatgacgaaaatatcacccacgaagacgatataaaggaaacagaccatgatggcgaatccgaagagcgagacactgtatctacaaagtccaagccgaagaaacagtccccaaaatcgtttgttggtgctcatttgaggaggcaactctcttttgatttatggtacctattccttggattatttataatatgcatatgcgagggcagaaaaatcgaagacgttaataaacctgatttcaatgtctttgctatattgtttgaagttgttagcgcttatggtacagtgggtttgtcattgggttacccaaacaccaacacatcactatctgcccagttcaccgtattatcgaagctagtcataattgccatgctaataagaggaagaaatagaggtttaccatacactttggatcgtgccatcatgctgccaagtgacaaactggaacaaattgatcgtttacaagatatgaaagctaagggtaagttgttagccaaagttggtgaggatccaatgactacttacgtcaaaaagagatcccacaaactgaaaaaaatagcaacaaagttttgggggaagcattaa

SEQ ID NO: 42 - protein, 889 aa, Saccharomyces cerevisiae
MPTAKRTSSRASLALPFQLRLVHKKSWGHRLRDFISGFLKSCRPIAKYVFPNFIVVHYIYLITLSIIGSILLYPCKNTAFIDVLFLAAGASTQGGLATKSTNDFNLYQQIVVYVITLLSTPILIHGFLAFVRLYWFERYFDNIRDISKQNFKLRRTMTLQQRELSGSSGNAARSRSFKDNLFRGKFVSREDPRQSASDVPMDSPDTSALSSISPLNVSSSKEESSDTQSSPPNFSSKRQPSDVDPRDIYKSIMMLQKQQEKSNANSTDSFSSETNGPAFIVQERHERRAPHCSLKRHSVLPSSQELNKLAQTKSFQKLLGLRRDEGDHDYFDGAPHKYMVTKKKKISRTQSCNIPTYTASPSPKTSGQVVENHRNLAKSAPSSFVDEEMSFSPQESLNLQFQAHPPKPKRREGDIGHPFTRTMSTNYLSWQPTFGRNSVFIGLTKQQKEELGGVEYRALRLLCCILMVYYIGFNILAFVTIVPWACTRHHYSEIIRRNGVSPTWWGFFTAMSAFSNLGLSLTADSMVSFDTAPYPLIFMMFFIIIGNTGFPIMLRFIIWIMFKTSRDLSQFKESLGFLLDHPRRCFTLLFPSGPTWWLFTTLVVLNATDWILFIILDFNSAVVRQVAKGYRALMGLFQSVCTRTAGFNVVDLSKLHPSIQVSYMLMMYVSVLPLAISIRRTNVYEEQSLGLYDSGQDDENITHEDDIKETDHDGESEERDTVSTKSKPKKQSPKSFVGAHLRRQLSFDLWYLFLGLFIICICEGRKIEDVNKPDFNVFAILFEVVSAYGTVGLSLGYPNTNTSLSAQFTVLSKLVIIAMLIRGRNRGLPYTLDRAIMLPSDKLEQIDRLQDMKAKGKLLAKVGEDPMTTYVKKRSHKLKKIATKFWGKH

SEQ ID NO: 43 - DNA, 3726 bp, Saccharomyces uvarum
atgcacattagagggaccatgagcagagtgcccacattagcatcgttcgaagtacgatacaaaaaatccttcggccacaaatttcgtgatttcattgctctatgtggccactattgctccccaattaaaaaatatattttccctaattttatcgcagtccattatttctatacgatagtcctcacgctaataacctctattctgttgtatccagttaagaacatcagatatatcgacgctttgtttttggcagcaggtgcggtcactcaaggtggtttgaatactgtggatgtcaacaatctaaccttatatcaacagattattttgtacattatatgctgcatatcaacgcccattgccgttcatagttgtttggcattcgtaagactctattggtttgaacgttattttgacggtattagagactcgtccaggctaaatttcaaaatgagaagaacaaagacaatcttggaaagggaactaaccgctagaaccatgaccaaaagtaagacaggagggactcaaagagtgtcacgtcccgggaaatcagacaagagagacgatttccaagaaaaattgtttaatggagaaatggttaatagagatgagcaggactcggttcatagtagccacaattctcgtgatagtaatagcaatgccaataccaatagtagcaacaataatagcatcaaccacaatggtagtagtggcagtttggatgattatgttagggaagacaaagcggatgaggtcgaaaaataccacggaaataagtcctattcaagtgtaggtagttcgtccaatacagccacagatgaaaatataagtcaaaaattaaagccaagtagtcttcgattcgatgagtcgcaaaataaaagaaaacgtacgggagccccttcagaaaaatttgccaaaaggagaggttcaagagatatcactcctgaagatatgtatcgatccattatgatgctacaaggtgagcacgagggtactgcagaagatgaaggtccacctttagttattggatcgcctacggatggtacaagaaatatggacaacggtagtgagtcaaagtctgctcccacgatgaatgagagtaaaatcaggattcaggataaaggagctaaaattagccttgaccaggactccgtattacatagttcaaattcttccgcatgcacttcggatgaggattctctacctacaaattttggaggcacaacaccttcattgagcgcaaagccacgggaatcaagttcaggcccaatagcgttcactgaaggggaaaatgctgatagaaaacaaggcccatcgattcagttcaatatcactaaaccgccaaggaaggcttctaaatcgaagagggtttctactatggatgacttgaacccaagatcaatttttccgcatcaaaaaaaatcatcgaaaggatacataatgaaacatttgccgaaagcccgtcgaatacggcagcagattaaaagaagactttccacgggttcgatcgataaaaacagtagcagtgacgctctcgatagagggctcatttctggtctaaatgatgatgatgatggcaacgaaggtgataacatggaagagtactttgccgacaacgaaagtggggatgaagatgaccgaatgcagcaatctgaaccacagtctggatcagaacttaaattgaaacaacagcaacagcaccaattgcagcaaaacctgcatcgtatgtacaagacgaaatcttttgacgataatcgttcaaaacctgtcctcatggaaaggtctaagactatcgatatggctgaagctagagatttaaatgaacttgccaggactcctgactttcaaaaaatggtttataagaattggaaagcccatcatagaaataaacctaacttcaaaagaaggggctggaatggcaagatgtttgaacatggtccttatacatctgacagcgaccataattatcaggataatggaaataatagtaatagtatagttcactacgcagaatctattctacatcgtgataattctcatagaaatggaagcgaagatgtttcttcagattctaatgagaccacctatcctttgaatggaaacaatgatcacagccaaaacgatgctaatggctatcccacctacaacgatgaagaggaaggctattacggtttacatttcgattctgattataacttggatcctcaccatgctctatccagtaacgctagcaaaaactacttgtcatggcaaccaaccattggacgtaattcaaactttcttgggttgacaagagcccaaaaggacgaattgggtggtgtcgagtacagagccatcaaactactatgtaccatattggttgtctattatgttggatggcacatcgtctcttttgttatgttggtgccttggattaacttgaaaaagcactatagcgatattgtcagaagcgatggcgtttcacctacatggtggggtttttggacagcaatgagtgccttcaacgatttgggattaacattaaccccggactcaatgatgtcatttgataaagctgtatatcccttgatcgttatgatttggtttatcattattggaaatacagggtttcccatccttcttagatgcattatttggataatgttcaaactttcccctgatttgtcacagatgagagagagtttaggctttcttttagatcacccgcgtcgttgttttactctattatttccaaaggctgccacatggtggctgcttttgacgcttgtgggactaaattttacagattggatcttatttattattttggattttggatcaactgtagttaaatcgttatcaaaaggttatagagttcttgttggtttattccaatctgtcagtacgaggaccgccggtttcagtgtcgttgatttgagtcagttacacccctctatccaagtctcctatatgcttatgatgtatgtctccgtgttgccattagctatttctattagacggacaaatgtttacgaggagcaatctttgggactgtatggagaaatgggaggtaaaccagaagatactgatactgaagaggacggcgactgcgatgatgaagatgacgacaacgaagaagaggagagccatgaaggtggaagtagccaaaggggcaaatcaaagaaagaaacaaaaaagaaaaaaaagagaaaagaaaatgagaaccccaacgaagaatcaaccaagtccttcattggtgcgcatttaagaagacagctatcttttgatttatggtttttgtttcttgggctatttatcatttgtatctgcgaacgtgacaagataaaagacatccagagaccgaacttcaatgtatttacaattctttttgagattgtaagcgcttatggtacagtcggactatcattgggctatccgaacactaaccaatcattttcaagacagttaaccacattatctaaattaatcatcatagctatgctgattagaggtaagaatagaggtctgccctattctttggaccgtgctattatcctgccaagtgatagacttgaacatattgatcacattgaggacttgaaattgaagagacaggccagaaccgatacggatgatccgatgacggagcatctcaagagaagcattagcgatgccaaacatcgttgggacgaacttaaacacaagagaagcctttcccggagttcgaaaagaagcaccaaaaccaactaa

SEQ ID NO: 44 - protein, 1241 aa, Saccharomyces uvarum
MHIRGTMSRVPTLASFEVRYKKSFGHKFRDFIALCGHYCSPIKKYIFPNFIAVHYFYTIVLTLITSILLYPVKNIRYIDALFLAAGAVTQGGLNTVDVNNLTLYQQIILYIICCISTPIAVHSCLAFVRLYWFERYFDGIRDSSRLNFKMRRTKTILERELTARTMTKSKTGGTQRVSRPGKSDKRDDFQEKLFNGEMVNRDEQDSVHSSHNSRDSNSNANTNSSNNNSINHNGSSGSLDDYVREDKADEVEKYHGNKSYSSVGSSSNTATDENISQKLKPSSLRFDESQNKRKRTGAPSEKFAKRRGSRDITPEDMYRSIMMLQGEHEGTAEDEGPPLVIGSPTDGTRNMDNGSESKSAPTMNESKIRIQDKGAKISLDQDSVLHSSNSSACTSDEDSLPTNFGGTTPSLSAKPRESSSGPIAFTEGENADRKQGPSIQFNITKPPRKASKSKRVSTMDDLNPRSIFPHQKKSSKGYIMKHLPKARRIRQQIKRRLSTGSIDKNSSSDALDRGLISGLNDDDDGNEGDNMEEYFADNESGDEDDRMQQSEPQSGSELKLKQQQQHQLQQNLHRMYKTKSFDDNRSKPVLMERSKTIDMAEARDLNELARTPDFQKMVYKNWKAHHRNKPNFKRRGWNGKMFEHGPYTSDSDHNYQDNGNNSNSIVHYAESILHRDNSHRNGSEDVSSDSNETTYPLNGNNDHSQNDANGYPTYNDEEEGYYGLHFDSDYNLDPHHALSSNASKNYLSWQPTIGRNSNFLGLTRAQKDELGGVEYRAIKLLCTILVVYYVGWHIVSFVMLVPWINLKKHYSDIVRSDGVSPTWWGFWTAMSAFNDLGLTLTPDSMMSFDKAVYPLIVMIWFIIIGNTGFPILLRCIIWIMFKLSPDLSQMRESLGFLLDHPRRCFTLLFPKAATWWLLLTLVGLNFTDWILFIILDFGSTVVKSLSKGYRVLVGLFQSVSTRTAGFSVVDLSQLHPSIQVSYMLMMYVSVLPLAISIRRTNVYEEQSLGLYGEMGGKPEDTDTEEDGDCDDEDDDNEEEESHEGGSSQRGKSKKETKKKKKRKENENPNEESTKSFIGAHLRRQLSFDLWFLFLGLFIICICERDKIKDIQRPNFNVFTILFEIVSAYGTVGLSLGYPNTNQSFSRQLTTLSKLIIIAMLIRGKNRGLPYSLDRAIILPSDRLEHIDHIEDLKLKRQARTDTDDPMTEHLKRSISDAKHRWDELKHKRSLSRSSKRSTKTN

SEQ ID NO: 45 - DNA, 1828 bp, Oryza sativa
gcttgagtcatagggagaaaacaaatcgatcatatttgactcttttccctccatctctcttaccggcaaaaaaagtagtactggtttatatgtaaagtaagattctttaattatgtgagatccggcttaatgcttttcttttgtcacatatactgcattgcaacaattgccatatattcacttctgccatcccattatatagcaactcaagaatggattgatatatcccctattactaatctagacatgttaaggctgagttgggcagtccatcttcccaacccaccaccttcgtttttcgcgcacatacttttcaaactactaaatggtgtgttttttaaaaatattttcaatacaaaagttgctttaaaaaattatattgatccatttttttaaaaaaaatagctaatacttaattaatcacgtgttaaaagaccgctccgttttgcgtgcaggagggataggttcacatcctgcattaccgaacacagcctaaatcttgttgtctagattcgtagtactggatatattaaatcatgttctaagttactatatactgagatgaatagaataagtaaaattagacccaccttaagtcttgatgaagttactactagctgcgtttgggaggacttcccaaaaaaaaaagtattagccattagcacgtgattaattaagtactagtttaaaaaacttaaaaaataaattaatatgattctcttaagtaactctcctatagaaaacttttacaaaattacaccgtttaatagtttggaaaatatgtcagtaaaaaataagagagtagaagttatgaaagttagaaaaagaattgttttagtagtatacagttataaactattccctctgttctaaaacataagggattatggatggattcgacatgtaccagtaccatgaatcgaatccagacaagttttttatgcatatttattctactataatatatcacatctgctctaaatatcttatatttcgaggtggagactgtcgctatgtttttctgcccgttgctaagcacacgccacccccgatgcggggacgcctctggccttcttgccacgataattgaatggaacttccacattcagattcgataggtgaccgtcgactccaagtgctttgcacaaaacaactccggcctcccggccaccagtcacacgactcacggcactaccacccctgactccctgaggcggacctgccactgttctgcatgcgaagctatctaaaattctgaagcaaagaaagcacagcacatgctccgggacacgcgccacccggcggaaaagggctcggtgtggcgatctcacagccgcatatcgcatttcacaagccgcccatctccaccggcttcacgaggctcatcgcggcacgaccgcgcacggaacgcacgcggccgacccgcgcgcctcgatgcgcgagcccatccgccgcgtcctccctttgcctttgccgctatcctctcggtcgtatcccgtttctctgtcttttgctccccggcgcgcgccagttcggagtaccagcgaaacccggacacctggtacacctccgccggccacaacgcgtgtcccccctacgtggccgcgcagcacatgcccatgcgcgacacgtgcacctcctcatccaaactctcaagtctcaacggtcctataaatgcacggatagcctcaagctgctcgtcacaaggcaagaggcaagaggcaagagcatccgtattaaccagccttttgagacttgagagtgtgtgtgactcgatccagcgtagtttcagttcgtgtgttggtgagtgattccagccaagtttgcg

(19) United States
(12) Patent Application Publication
(10) Pub. No.: US 2008/0108502 A1
(43) Pub. Date: May 8, 2008

(54) Plants Having Improved Growth Characteristics And Method For Making The Same

(75) Inventor: Ana Isabel Sanz Molinero, Gentbrugge (BE)

Correspondence Address: CONNOLLY BOVE LODGE & HUTZ, LLP, P O BOX 2207, WILMINGTON, DE, 19899, US

(73) Assignee: CropDesign N.V., Zwijnaarde (BE)

(21) Appl. No.: 11/666,582

(22) PCT Filed: Oct. 27, 2005
(86) PCT No.: PCT/EP05/55590

§ 371 Date: Oct. 11, 2007

Related U.S. Application Data
(60) Provisional application No. 60/624,892, filed on Nov. 4, 2004.

(30) Foreign Application Priority Data
Oct. 29, 2004 (EP) 04105406.5

(51) Int. Cl. A01N 33/00; A01H 5/00; C07H 21/04; C12N 5/02; C12Q 1/68

(52) U.S. Cl. 504/326; 435/419; 435/6; 536/24.1; 800/290; 800/298

(57) ABSTRACT

The present invention concerns a method for improving growth characteristics of plants by increasing activity in a plant of an HKT protein or a homologue thereof. One such method comprises introducing into a plant an HKT nucleic acid molecule or functional variant thereof. The invention also relates to transgenic plants having improved growth characteristics, which plants have increased expression of a nucleic acid encoding an HKT protein. The present invention also concerns constructs useful in the methods of the invention.

The present invention relates generally to the field of molecular biology and concerns a method for improving plant growth characteristics. More specifically, the present invention concerns a method for improving plant growth characteristics, in particular for increasing yield, by increasing activity in a plant of a Na⁺-K⁺ cotransporter protein (HKT) or a homologue thereof. The present invention also concerns plants having increased HKT activity, which plants have improved growth characteristics relative to corresponding wild type plants. The invention also provides constructs useful in the methods of the invention.

Given the ever-increasing world population, and the dwindling area of land available for agriculture, it remains a major goal of agricultural research to improve the efficiency of agriculture and to increase the diversity of plants in horticulture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic complements that may not always result in the desirable trait being passed on from parent plants. Advances in molecular biology have allowed mankind to manipulate the germplasm of animals and plants. Genetic engineering of plants entails the isolation and manipulation of genetic material (typically in the form of DNA or RNA) and the subsequent introduction of that genetic material into a plant. Such technology has led to the development of plants having various improved economic, agronomic or horticultural traits. Traits of particular economic interest are growth characteristics such as high yield. Yield is normally defined as the measurable produce of economic value from a crop. This may be defined in terms of quantity and/or quality. Yield is directly dependent on several factors, for example, the number and size of the organs, plant architecture (for example, the number of branches), seed production and more. Root development, nutrient uptake and stress tolerance may also be important factors in determining yield. Crop yield may therefore be increased by optimising one of the above-mentioned factors.

A major abiotic stress factor for plants is salinity. Salt stress, and in particular Na⁺ stress, may negatively influence several cellular processes. Besides hyperosmotic damage, which comprises membrane dysfunction, high sodium concentrations may interfere with Na⁺ sensitive enzymes and may result in distorted ion transport (for an overview of cellular and molecular responses, see Hasegawa et al., 2000. Annu. Rev. Plant Physiol. Plant Mol. Biol. 51, 463-499). On the other hand, potassium is an important nutrient, necessary for neutralizing negative charges on proteins, activation of K⁺-dependent enzymes, maintenance of cell turgor and osmotic homeostasis. Na⁺ and K⁺ transport are linked to each other: experimental data indicate that both ions use the same transport proteins and plants may compensate shortage of potassium by taking up sodium (Pitman, 1967. Nature 216, 1343-1344; Pitman et al., 1968. Aust. J. Biol. Sci 21, 871-881). Voltage-insensitive monovalent-cation channels (VIC) play an important role in the uptake of Na⁺ and K⁺ in plant cells (White, 1999. Trends Plant Sci. 4, 245-246). In addition, several K⁺ up taking proteins have been described in Arabidopsis (Maser et al., 2001. Plant Physiol. 126:1646-1667): AKT/KAT-type channel proteins, HAK/AT/KUP-like transporter proteins and HKT-type transporter proteins. Plant HKT proteins are part of a superfamily of cation transporters that also comprise the yeast Trk proteins and prokaryotic KdpA, KrkH and KtrB proteins (Schachtman & Liu, 1999. Trends Plant Sci. 4, 281-286).

The transmembrane protein AtHKT1 was shown to be also involved in Na⁺ uptake in roots and in salt tolerance in Arabidopsis (Uozumi et al., 2000. Plant Physiol. 122, 1249-1259; Rus et al., 2001. Proc. Natl. Acad. Sci. USA 98, 14150-14155); its rice counterpart (OsHKT1) fulfilled a similar function under conditions of potassium deprivation (Garciadeblás et al, 2003. Plant J. 34, 788-801). AtHKT1 was also reported to be important in recirculation of Na⁺ from the shoots to the roots, thereby contributing to plant salt tolerance (Berthomieu et al, 2003. EMBO J. 22, 2004-2014). In rice, 9 types of HKT transporters have been identified and characterised (Garciadeblás et al, 2003. Plant J. 34, 788-801, Horie et al, 2001. Plant J. 27, 129-138). In addition, rice HKT1 not only mediated Na⁺ and K⁺ transport, but also mediated transport of other alkali cations (Golldack et al., 2002. Plant J. 31, 529-542). The effect of high and low HKT1 protein levels was studied in rice (Golldack et al., 2002): the salt tolerant line Pokkali and the salt sensitive line IR29 differed significantly from each other in their ability to take up or exclude sodium. High concentrations of alkali ions repressed OsHKT1 expression in both lines, but this repression was less pronounced in root and leaf vascular tissues of the IR29 line than in the salt tolerant line Pokkali.

Few data are available with respect to heterologous expression of HKT1 genes in plants. Schroeder and Schachtman (WO96/05722) disclose an HKT1 protein from wheat and suggested use of this HKT1 protein to modulate salt uptake from the environment by manipulating its expression, resulting in plants that can be used for desalinization (increased sodium uptake upon enhanced HKT1 expression) or in plants that are resistant to toxic alkali metals (decreased or inhibited uptake by repressing HKT1 expression). However as shown by Laurie et al. (Plant J. 32, 139-149, 2002), plants overexpressing HKT1 had a phenotype that was not much different from the control plants: under NaCl stress, the fresh weight of the HKT1 overexpressing line was reduced to a similar degree as for the control plants and there was no significant increase in the Na⁺ content of roots, compared to the control. Laurie et al. have also shown that the conditions for obtaining downregulated expression of HKT1 are quite complex. So far, the prior art relating to HKT1 was mainly focused on cation transport and ion homeostasis, and many studies were performed in yeast or Xenopus oocytes, which results may not be fully reflective for HKT1 function in plants.

AtHKT1 is a protein encoded by a single gene in Arabidopsis thaliana. The gene is thought to be present in all plant genomes, and may sometimes occur as a small gene family, for example as in rice. The AtHKT1 gene is expressed in roots and to a lesser extent in other tissues (Uozumi et al., 2000). A three dimensional model of an HKT type protein was constructed using sequence and hydrophobicity analysis (Durell et al., Biophys. J. 77, 775-788, 1999; Durell and Guy, Biophys. J. 77, 789-807, 1999): the protein was shown to be hydrophobic and comprised a core structure of eight transmembrane domains flanking four loops that formed a pore (in other words: four units of a transmembrane domain—pore forming domain—transmembrane domain), each pore forming domain was predicted to be located partly inside the membrane (see FIG. 1) and comprised a conserved Glycine or Serine residue. Heterologous expression of wild type AtHKT1 revealed this protein to selectively mediate Na⁺ transport while K⁺ transport occurred to a lesser degree (Uozumi et al., 2000). Other HKT proteins from different plant species show a Na⁺/K⁺ symporter activity. The ion selectivity is believed to be determined by the first pore forming domain (or P-loop). Mutating Ser-68 to Glycine in the AtHKT1 gene restored the permeability for K⁺. Glycine residues corresponding to the mutated Gly-68 of AtHKT1 were found in other HKT proteins; it was shown that this is the first Gly in a conserved “GYG” motif that functions as an ion selectivity filter (Mäser et al., Proc. Natl. Acad. Sci. USA 99, 6428-6433, 2002). In addition to Na⁺ and K⁺, HKT type proteins were also shown to transport other cations like Rb⁺, Li⁺ and Cs⁺ (Golldack et al., 2002). Besides Arabidopsis, HKT proteins were isolated from various other plant species, including rice, wheat, Eucalyptus camaldulensis, Hordeum vulgare and Mesembryanthemum crystallinum.

It has now surprisingly been found that increasing activity of an HKT protein or a homologue thereof in a plant gives rise to plants having improved growth characteristics compared to wild type plants, in particular under non-stress conditions.

According to one embodiment of the present invention, there is provided a method for improving the growth characteristics of a plant, comprising increasing activity of an HKT protein or a homologue thereof in a plant. Optionally there may follow a step for selecting plants having improved growth characteristics.

Advantageously, performance of the methods according to the present invention results in plants having a variety of improved growth characteristics, especially increased yield, particularly seed yield.

The term “increased yield” as defined herein is taken to mean an increase in any one or more of the following, each relative to corresponding wild type plants: (i) increased biomass (weight) of one or more parts of a plant, particularly aboveground (harvestable) parts, increased root biomass or increased biomass of any other harvestable part; (ii) increased total seed yield, which includes an increase in seed biomass (seed weight) and which may be an increase in the seed weight per plant or on an individual seed basis; (iii) increased number of flowers (“florets”) per panicle (iv) increased number of (filled) seeds; (v) increased seed size, which may also influence the composition of seeds; (vi) increased seed volume, which may also influence the composition of seeds (including oil, protein and carbohydrate total content and composition); (vii) increased individual seed area; (viii) increased individual seed length and/or width; (ix) increased harvest index, which is expressed as a ratio of the yield of harvestable parts, such as seeds, over the total biomass; and (x) increased thousand kernel weight (TKW), which is extrapolated from the number of filled seeds counted and their total weight. An increased TKW may result from an increased seed size and/or seed weight. An increased TKW may result from an increase in embryo size and/or endosperm size.

Taking corn as an example, a yield increase may be manifested as one or more of the following: increase in the number of plants per hectare or acre, an increase in the number of ears per plant, an increase in the number of rows, number of kernels per row, kernel weight, TKW, ear length/diameter, among others. Taking rice as an example, a yield increase may be manifested by an increase in one or more of the following: number of plants per hectare or acre, number of panicles per plant, number of spikelets per panicle, number of flowers per panicle, increase in the seed filling rate, increase in TKW, among others. An increase in yield may also result in modified architecture, or may occur as a result of modified architecture.

According to a preferred feature, performance of the methods according to the present invention results in plants having increased yield and more particularly, increased biomass and/or increased seed yield. Therefore, according to the present invention, there is provided a method for improving growth characteristics of a plant, in particular for increasing plant yield, which method comprises increasing activity of an HKT protein or a homologue thereof in a plant.

Since the improved plants according to the present invention have increased yield, it is likely that these plants exhibit an increased growth rate (during at least part of their life cycle), relative to the growth rate of corresponding wild type plants at a corresponding stage in their life cycle. The increased growth rate may be specific to one or more parts or cell types of a plant (including seeds), or may be throughout substantially the whole plant. Plants having an increased growth rate may have a shorter life cycle. The life cycle of a plant is taken to mean the time needed to grow from a dry mature seed up to the stage where the plant has produced dry mature seeds similar to the starting material. This life cycle may be influenced by factors such as early vigour, growth rate, flowering time and speed of seed maturation. An increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible. If the growth rate is sufficiently increased, it may allow for the sowing of further seeds of the same plant species (for example sowing and harvesting of rice plants followed by sowing and harvesting of further rice plants all within one conventional growing period). Similarly, if the growth rate is sufficiently increased, it may allow for the sowing of further seeds of different plants species (for example the sowing and harvesting of rice plants followed by, for example, the sowing and optional harvesting of soy bean, potatoes or any other suitable plant). Harvesting additional times from the same rootstock in the case of some plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves plotting growth experiments, such parameters may be: T-Mid (the time taken for plants to reach 50% of their maximal size) and T-90 (time taken for plants to reach 90% of their maximal size), amongst others.

Performance of the methods of the invention gives plants having an increased growth rate. Therefore, according to the present invention, there is provided a method for increasing the growth rate of plants, which method comprises increasing activity of an HKT protein or a homologue thereof in a plant.

An increase in yield and/or growth rate occurs whether the plant is under non-stress conditions or whether the plant is exposed to various stresses compared to control plants. It should thus be understood that in the present invention the increase in yield and/or growth rate is not dependent on the growth conditions, whether they be stress conditions or non-stress conditions. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the typical stresses to which a plant may be exposed. These stresses may be the everyday biotic and/or abiotic (environmental) stresses to which a plant is exposed. Typical abiotic or environmental stresses include temperature stresses caused by atypical hot or cold/freezing temperatures; salt stress; water stress (drought or excess water). Abiotic stresses may also be caused by chemicals. Biotic stresses are typically those stresses caused by pathogens, such as bacteria, viruses, fungi and insects. The term “non-stress conditions” as used herein are those environmental conditions that do not significantly go beyond the everyday climatic and other abiotic stress conditions that plants may encounter. Persons skilled in the art are aware of normal soil conditions and climatic conditions for a given geographic location.

The abovementioned growth characteristics may advantageously be improved in any plant.

The term “plant” as used herein encompasses whole plants, ancestors and progeny of the plants and plant parts, including seeds, shoots, stems, leaves, roots (including tubers), flowers, and tissues and organs. The term “plant” also encompasses plant cells, suspension cultures, callus tissue, embryos, meristematic regions, gametophytes, sporophytes, pollen, and microspores. The term “plant” furthermore encompasses plants, plant parts or plant cells modified by human intervention, such as mutated or transformed plants or plant parts, which mutated or transformed plants or plant parts comprise the gene/nucleic acid of interest or the specific modification in the gene/nucleic acid of interest.

Plants that are particularly useful in the methods of the invention include algae, ferns, and all plants which belong to the superfamily Viridiplantae, in particular monocotyledonous and dicotyledonous plants, including fodder or forage legumes, ornamental plants, food crops, trees, or shrubs selected from the list comprising Abelmoschus spp., Acer spp., Actinidia spp., Agropyron spp., Allium spp., Amaranthus spp., Ananas comosus, Annona spp., Apium graveolens, Arabidopsis thaliana, Arachis spp, Artocarpus spp., Asparagus officinalis, Avena sativa, Averrhoa carambola, Benincasa hispida, Bertholletia excelsea, Beta vulgaris, Brassica spp., Cadaba farinosa, Camellia sinensis, Canna indica, Capsicum spp., Carica papaya, Carissa macrocarpa, Carthamus tinctorius, Carya spp., Castanea spp., Cichorium endivia, Cinnamomum spp., Citrullus lanatus, Citrus spp., Cocos spp., Coffea spp., Cola spp., Colocasia esculenta, Corylus spp., Crataegus spp., Cucumis spp., Cucurbita spp., Cynara spp., Daucus carota, Desmodium spp., Dimocarpus longan, Dioscorea spp., Diospyros spp., Echinochloa spp., Eleusine coracana, Eriobotrya japonica, Eugenia uniflora, Fagopyrum spp., Fagus spp., Ficus carica, Fortunella spp., Fragaria spp., Ginkgo biloba, Glycine spp., Gossypium hirsutum, Helianthus spp., Hibiscus spp., Hordeum spp., Ipomoea batatas, Juglans spp., Lactuca sativa, Lathyrus spp., Lemna spp., Lens culinaris, Linum usitatissimum, Litchi chinensis, Lotus spp., Luffa acutangula, Lupinus spp., Macrotyloma spp., Malpighia emarginata, Malus spp., Mammea americana, Mangifera indica, Manihot spp., Manilkara zapota, Medicago sativa, Melilotus spp., Mentha spp., Momordica spp., Morus nigra, Musa spp., Nicotiana spp., Olea spp., Opuntia spp., Ornithopus spp., Oryza spp., Panicum miliaceum, Passiflora edulis, Pastinaca sativa, Persea spp., Petroselinum crispum, Phaseolus spp., Phoenix spp., Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Rubus spp., Saccharum spp., Sambucus spp., Secale cereale, Sesamum spp., Solanum spp., Sorghum bicolor, Spinacia spp., Syzygium spp., Tamarindus indica, Theobroma cacao, Trifolium spp., Triticosecale rimpaul, Triticum spp., Vaccinium spp., Vicia spp., Vigna spp., Vitis spp., Zea mays, Zizania palustris, Ziziphus spp., amongst others.

According to a preferred feature of the present invention, the plant is a crop plant such as soybean, sunflower, canola, alfalfa, rapeseed or cotton. Further preferably, the plant according to the present invention is a monocotyledonous plant such as sugarcane, most preferably a cereal, such as rice, maize, wheat, millet, barley, rye, oats or sorghum.

The activity of an HKT protein or a homologue thereof may be increased by increasing levels of the HKT polypeptide, for example by increasing the level of nucleic acids encoding an HKT protein. Alternatively, activity may also be increased when there is no change in levels of an HKT, or even when there is a reduction in levels of an HKT. This may occur when the intrinsic properties of the polypeptide are altered, for example, by making a mutant or selecting a variant that is more active than the wild type.

The term “HKT protein or homologue thereof” as defined herein refers to a polypeptide with HKT activity and which polypeptide comprises four units of a transmembrane domain—pore forming domain—transmembrane domain. Preferably, the HKT protein has a sequence as represented by SEQ ID NO: 2, or is a homologue thereof having in increasing order of preference at least 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95% overall sequence identity to the amino acid represented by SEQ ID NO: 2. The overall sequence identity is determined using an alignment algorithm that can perform global alignments, such as the Needleman Wunsch algorithm in the program GAP (GCG Wisconsin Package, Accelrys).

An “HKT protein or a homologue thereof” falling within the above definition may readily be identified using routine techniques well known to persons skilled in the art. For example, HKT acitivity as cation transporter may readily be determined in vitro or in vivo using techniques well known in the art. For example, the complementation and the effect on K⁺ uptake upon cloning of an HKT gene in the Escherichia coli strain LB2003 (deficient for the K⁺ uptake systems Trk, Kup and Kdp) may be assayed as described by Uozumi et al. (2000). Similarly, the Saccharomyces cerevisiae strain CY162 (deficient for the high affinity uptake systems trk1 and trk2) may be complemented by cloning an HKT gene, whereas the yeast strain G19 (deficient for the Na⁺ extruding ATPase genes ENA1 to ENA4) or a wild type yeast strain will exhibit growth inhibition upon cloning of an HKT gene (Horie et al., 2001). Alternatively, a voltage clamp assay may be performed using Xenopus laevis oocytes (see for example Horie et al., 2001) or a cation uptake/depletion test in roots or yeast as described by Garciadeblas et al. (Plant J. 34, 788-801, 2003) and Banuelos et al. (Plant Physiol. 130, 784-795, 2002). At least one of the above mentioned assays (or other assays known in the art) will demonstrate the cation transporting activity of an HKT protein or a homologue thereof. Alternatively, such “HKT protein or homologue thereof”, when expressed under control of a WSI18 promoter in the Oryza sativa cultivar Nipponbare, increases seed yield compared to corresponding wild type plants. This increase in seed yield may be measured in several ways, for example as an increase of the number of filled seeds. Increased HKT activity thus encompasses at least one of increased levels of a nucleic acid encoding an HKT protein or a homologue thereof, increased levels of an HKT protein or a homologue thereof, increased transporter activity or increased seed yield.

Techniques for measuring increased levels of an HKT encoding nucleic acid are known in the art and include for example Northern Blotting, Real Time-PCR or Quantitative-PCR. Increased HKT protein levels may be determined using for example Western Blotting, by estimating band/spot intensity after gel electrophoresis of a crude protein sample, or by testing enzymatic activity (if applicable).

The various structural domains in an HKT protein may be identified using specialised databases e.g. SMART (Schultz et al. (1998) Proc. Natl. Acad. Sci. USA 95, 5857-5864; Letunic et al. (2002) Nucleic Acids Res 30, 242-244; http://smart.embl-heidelberg.de/), InterPro (Mulder et al., (2003) Nucl. Acids. Res. 31, 315-318; http://www.ebi.ac.uk/interpro/), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp53-61, AAAIPress, Menlo Park; Hulo et al., Nucl. Acids. Res. 32:D134-D137, (2004), http://www.expasy.org/prosite/) or Pfam (Bateman et al., Nucleic Acids Research 30(1):276-280 (2002), http://www.sanger.ac.uk/Software/Pfam/).

The structural domains in an HKT protein are known in the art and the topological model is supported by experimental evidence (Kato et al., Proc. Natl. Acad. Sci. USA 98, 6488-6493, 2001). The HKT group of proteins consists of proteins comprising four “transmembrane domain—pore domain—transmembrane domain” units (MPM). Each of the four pore forming domains comprises a conserved Glycine or Serine residue. The conserved Glycine (or Serine) residue in the first pore forming domain (indicated by an asterisk in domain A in FIG. 1) determines cation specificity. Each MPM unit comprises an MPM motif as characterised by Durell et al. (1999). For example, the fourth MPM unit in AtHKT1 (SEQ ID NO: 2) comprises the motif:

GLIVSQLSFLTICIFLISITERQNLQRDPINFNVLNITLEVISAYGNVGFTTGYSCERRLDISDGGCKD ASYGFAGRWSPMGKFVLIIVMFYGRFKQFTAKSGRAWILYPSS

(SEQ ID NO: 7), wherein the bold underlined parts of the sequence represent the transmembrane motif and the underlined part in italics indicates the pore-forming loop motif, with the conserved Glycine residue in bold italics.

HKT proteins, such as SEQ ID NO: 2, also comprise a TrkH domain (Pfam accession number PF02386, Interpro accession IPR003445) (starting at G145 and ending with Y502 for SEQ ID NO: 2), which is characteristic for a group of proteins comprising potassium transport proteins (Trk) and V-type sodium ATP synthase subunit J or translocating ATPase J.

Preferably, the HKT protein useful in the present invention comprises in the fourth pore-forming domain a consensus sequence corresponding to EVISAYGNVGFTTGY (SEQ ID NO: 8) wherein the residues indicated in bold are invariably conserved and wherein the other residues may vary (see for example FIG. 2). Preferably, the consensus sequence in the fourth pore-forming domain corresponds to

(SEQ ID NO: 9)

E(V, I) (I, V) SA(Y, F) G(N, T) (V, A, I) G(F, L,
Y) (T, S) (T, I, L, V, M) GY.

More preferably, the consensus sequence in the fourth pore-forming domain corresponds to

E(V, I) (I, V) SA(Y, F) GNVG(F, L, Y) (T, S) (T,
L, V) GY.

Alternatively, the HKT protein useful in the methods of the present invention comprises in the fourth pore-forming domain an SA(Y,F)GN sequence signature and a DP(I,L)N(Y,F,L) sequence signature (SEQ ID NO: 10). Preferably, the signature sequence of SEQ ID NO: 10 is DP(I,L)NF.

Whether a polypeptide has at least 35% identity to the amino acid represented by SEQ ID NO: 2 may readily be established by sequence alignment. Methods for the search and identification of HKT homologues would be well within the realm of persons skilled in the art. Such methods comprise comparison of the sequences represented by SEQ ID NO: 1 or SEQ ID NO: 2, in a computer readable format, with sequences that are available in public databases such as MIPS (http://mips.gsf.de/), GenBank (http://www.ncbi.nim.nih.gov/Genbank/index.html) or the EMBL Nucleotide Sequence Database (http://www.ebi.ac.uk/embl/index.html), using algorithms well known in the art for the alignment or comparison of sequences, such as GAP (Needleman and Wunsch, J. Mol. Biol. 48; 443-453 (1970)), BESTFIT (using the local homology algorithm of Smith and Waterman (Advances in Applied Mathematics 2; 482-489 (1981))), BLAST (Altschul, S. F., Gish, W., Miller, W., Myers, E. W. & Lipman, D. J., J. Mol. Biol. 215:403-410 (1990)), FASTA and TFASTA (W. R. Pearson and D. J. Lipman Proc.Natl.Acad.Sci. USA 85:2444-2448 (1988)), or using, for example, the VNTI AlignX multiple alignment program, based on a modified Clustal W algorithm (InforMax, Bethesda, Md., http://www.informaxinc.com), with default settings for gap opening penalty of 10 and a gap extension of 0.05. The software for performing BLAST analysis is publicly available through the National Centre for Biotechnology Information (NCBI). The homologues mentioned below were identified using BLAST default parameters (BLOSUM62 matrix, gap opening penalty 11 and gap extension penalty 1) and preferably the full-length sequences are used for analysis.

Examples of polypeptides falling under the definition of an “HKT or a homologue thereof” include two Eucalyptus camaldulensis homologues (SEQ ID NO: 16, GenBank Accession No. AAF97728 and SEQ ID NO: 18, GenBank Accession No. AAD53890); two homologues from Mesembryanthemum crystallinum (SEQ ID NO: 14, GenBank Accession No. AAK52962 and SEQ ID NO: 12, GenBank Accession No. AAO73474). Other homologues suitable for practising the method according to the invention include the rice homologues represented in GenBank Accessions AAG37274 (OsHKT1, SEQ ID NO: 20), BAB61791 (OsHKT2, SEQ ID NO: 22), CAD37187 (OsHKT3, SEQ ID NO: 24), CAD37183 (OsHKT4, SEQ ID NO: 26), CAD37185 (OsHKT6, SEQ ID NO: 28), CAD37197 (OsHKT7, SEQ ID NO: 30), BAB93392 (OsHKT8, SEQ ID NO: 32) and CAD37199 (OsHKT9, SEQ ID NO: 34), and a wheat homologue (GenBank Accession No. AAA52749, SEQ ID NO: 36). It may however be envisaged that HKT homologues with a sequence identity lower than 35% to SEQ ID NO: 2 may still be suitable in the methods of the present invention, examples of such proteins are a Suaeda maritima homologue (SEQ ID NO: 38, GenBank Accession No. AY530754), or non-plant homologues such as yeast TRK1 (SEQ ID NO: 40, GenBank Accession No. NP_{—}012406) or TRK2 (SEQ ID NO: 42, GenBank Accession No. CAA82128) or Sacharomyces uvarum TRK1 (GenBank Accession No. JU0466 or AAA34661).

Table 1 lists examples of HKT proteins and homologues from other organisms, the length of the protein and their sequence identity to SEQ ID NO: 2.

TABLE 1

| | | |
|---|---|---|
| | | % identity |
| | Length | to AAF68393- |
| Protein sequence | aa | AtHKT1 |

| | | | |
|---|---|---|---|
| SEQ ID NO: 2 | AAF68393-AtHKT1 | 506 | 100.0 |
| SEQ ID NO: 12 | AAO73474-McHKT2 | 543 | 43.2 |
| SEQ ID NO: 14 | AAK52962-McHKT1 | 505 | 43.0 |
| SEQ ID NO: 16 | AAF97728-EcHKT1 | 550 | 46.0 |
| SEQ ID NO: 18 | AAD53890-EcHKT2 | 549 | 45.5 |
| SEQ ID NO: 36 | AAA52749-TaHKT1 | 533 | 36.9 |
| SEQ ID NO: 20 | AAG37274-OsHKT1 | 530 | 36.9 |
| SEQ ID NO: 22 | BAB61791-OsHKT2 | 530 | 37.0 |
| SEQ ID NO: 24 | CAD37187-OsHKT3 | 509 | 35.1 |
| SEQ ID NO: 26 | CAD37183-OsHKT4 | 552 | 37.1 |
| SEQ ID NO: 28 | CAD37185-OsHKT6 | 531 | 43.1 |
| SEQ ID NO: 30 | CAD37197-OsHKT7 | 500 | 42.2 |
| SEQ ID NO: 32 | BAB93392-OsHKT8 | 554 | 42.0 |
| SEQ ID NO: 34 | CAD37199-OsHKT9 | 509 | 35.1 |
| SEQ ID NO: 38 | AY530754-SmHKT1 | 485 | 33.5 |
| SEQ ID NO: 40 | NP_012406-ScTRK1 | 1235 | 11.3 |
| SEQ ID NO: 42 | CAA82128-ScTRK2 | 889 | 13.9 |
| SEQ ID NO: 44 | JU0466-SuTRK1 | 1241 | 9.7 |

At, Arabidopsis thaliana; Mc, Mesembryanthemum crystallinum; Ec, Eucalyptus camaldulensis; Ta, Triticum aestivum; Os, Oryza sativa; Sc, Saccharomyces cerevisiae; Sm, Suaeda maritime; Su, Saccharomyces uvarum

Despite what may appear to be a relatively low sequence homology to SEQ ID NO: 2 (as low as approximately 35%), HKT proteins are highly conserved in structure, with full-length proteins having four “transmembrane domain—pore domain—transmembrane domain” (MPM) units and preferably comprise in the fourth pore forming domain a consensus sequence corresponding to EVISAYGNVGFTTGY (SEQ ID NO: 8) wherein the residues indicated in bold are invariably conserved and wherein the other residues may vary. Preferably, the consensus sequence in the fourth pore-forming domain corresponds to SEQ ID NO: 9. More preferably, the consensus sequence in the fourth pore-forming domain corresponds to E(V,I)(I,V)SA(Y,F)GNVG(F,L,Y)(T,S)(T,L,V)GY. Alternatively, the HKT protein useful in the present invention comprises in the fourth pore-forming domain an SA(Y,F)GN sequence and a DP(I,L)N(Y,F,L) sequence. Furthermore, when HKT sequences from different sources are compared, conserved amino acids may be identified throughout the sequence (FIG. 2, which does not represent a limiting list of HKT proteins) and sequence identity within the fourth MPM unit among different species can be as high as 70%. HKT genes may therefore easily be found in other plant species. It is therefore to be understood that the term “HKT polypeptide or a homologue thereof” is not limited to the sequences represented by SEQ ID NO: 2 nor to SEQ ID NO: 12 to 36 listed above, but that any polypeptide meeting the criteria of having HKT activity and having an HKT topology as outlined above and having at least 35% sequence identity to SEQ ID NO: 2 may be suitable for use in the methods of the invention. As pointed out above, it may however be envisaged that proteins having the HKT topology outlined above but having a sequence identity lower than 35% to SEQ ID NO: 2 may still be useful in the methods of the present invention.

The nucleic acid encoding an HKT polypeptide or a homologue thereof may be any natural or synthetic nucleic acid. An HKT polypeptide or a homologue thereof as defined hereinabove is encoded by an HKT nucleic acid/gene. Therefore the term “HKT nucleic acid/gene” as defined herein is any nucleic acid/gene encoding an HKT polypeptide or a homologue thereof as defined hereinabove. Examples of HKT nucleic acids include those encoding SEQ ID NO: 2 and the sequence represented by SEQ ID NO: 1, or nucleic acids encoding proteins represented by SEQ ID NO: 12 to 36 (with the GenBank accession numbers AAF97728, AAD53890, AAK52962, AAO73474, AAG37274, BAB61791, CAD37187, CAD37183, CAD37185, CAD37197, BAB93392, CAD37199, or AAA52749. However nucleic acids encoding proteins having the HKT topology outlined above but having a sequence identity lower than 35% to SEQ ID NO: 2 may also be useful in the methods of the present invention; examples include SEQ ID NO: 38 to 44. HKT nucleic acids/genes and functional variants thereof may be suitable in practising the methods of the invention. Functional variant HKT nucleic acid/genes include portions of an HKT nucleic acid/gene and/or nucleic acids capable of hybridising with an HKT nucleic acid/gene. The term “functional” in the context of a functional variant refers to a variant (i.e. a portion or a hybridising sequence) which encodes a polypeptide having HKT activity and which polypeptide comprises four units of a transmembrane domain—pore forming domain—transmembrane domain, wherein the fourth pore forming domain preferably comprises a consensus sequence corresponding to EVISAYGNVGFTTGY (SEQ ID NO: 8) wherein the residues indicated in bold are invariably conserved and wherein the other residues may vary. Preferably, the consensus sequence in the fourth pore-forming domain corresponds to SEQ ID NO: 9. More preferably, the consensus sequence in the fourth pore-forming domain corresponds to E(V,I)(I,V)SA(Y,F)GNVG(F,L,Y)(T,S)(T,L,V)GY. Alternatively, the variant HKT protein may comprise in the fourth pore-forming domain an SA(Y,F)GN sequence and a DP(I,L)N(Y,F,L) sequence.

The term portion as defined herein refers to a piece of DNA comprising at least 80 nucleotides or more, preferably at least 330 nucleotides, which portion encodes a polypeptide having HKT activity and which polypeptide comprises at least one unit of a transmembrane domain—pore forming domain—transmembrane domain. A portion may be prepared, for example, by making one or more deletions to an HKT nucleic acid. The portions may be used in isolated form or they may be fused to other coding (or non coding) sequences in order to, for example, produce a protein that combines several activities, one of them being HKT activity, such as for example cation transporter activity. When fused to other coding sequences, the resulting polypeptide produced upon translation could be bigger than that predicted for the HKT fragment. Preferably, the functional portion is a portion of a nucleic acid as represented by SEQ ID NO: 1, or a portion of nucleic acids encoding proteins as defined above, and which portion encodes a polypeptide having HKT activity.

Another type of variant HKT is a nucleic acid capable of hybridising under reduced stringency conditions, preferably under stringent conditions, with an HKT nucleic acid/gene as hereinbefore defined, which hybridising sequence encodes a polypeptide having HKT activity and which polypeptide comprises at least one unit of a transmembrane domain—pore forming domain—transmembrane domain. The hybridising sequence is preferably at least 80 nucleotides in length, more preferably at least 330 nucleotides in length. Preferably, the hybridising sequence is capable of hybridising to a nucleic acid as represented by SEQ ID NO: 1, or to nucleic acids encoding proteins as defined above, such as the nucleic acids encoding proteins represented by SEQ ID NO: 2, or SEQ ID NO: 12 to SEQ ID NO: 36.

The term “hybridisation” as defined herein is a process wherein substantially homologous complementary nucleotide sequences anneal to each other, such that the sense strand of one species will anneal to the antisense strand of the other species. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration, ionic strength and hybridisation buffer composition.

“Stringent hybridisation conditions” and “stringent hybridisation wash conditions” in the context of nucleic acid hybridisation experiments such as Southern and Northern hybridisations are sequence dependent and are different under different environmental parameters. The skilled artisan is aware of various parameters which may be altered during hybridisation and washing and which will either maintain or change the stringency conditions.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4 M. Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:
DNA-DNA hybrids (Meinkoth and Wahl, Anal. Biochem., 138: 267-284,1984):
   Tₘ=81.5° C.+16.6×log[Na⁺]^{a}+0.41×%[G/C^{b}]−500×[L^{c}]⁻¹−0.61×% formamide
DNA-RNA or RNA-RNA hybrids:
   Tₘ=79.8+18.5(log₁₀[Na⁺]^{a})+0.58(% G/C^{b})+11.8(% G/C^{b})²−820/L^{c}
oligo-DNA or oligo-RNA^{d} hybrids:
   For <20 nucleotides: Tₘ=2 (lₙ)
   For 20-35 nucleotides: Tₘ=22+1.46 (lₙ)
^{a} or for other monovalent cation, but only accurate in the 0.01-0.4 M range.
^{b} only accurate for % GC in the 30% to 75% range.
^{c} L=length of duplex in base pairs.
^{d} Oligo, oligonucleotide; lₙ, effective length of primer=2×(no. of G/C)+(no. of A/T).

Note: for each 1% formamide, the Tₘ is reduced by about 0.6 to 0.7° C., while the presence of 6 M urea reduces the Tₘ by about 30° C.

Specificity of hybridisation is typically the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. Generally, suitable stringent conditions for nucleic acid hybridisation assays or gene amplification detection procedures are as set forth above. More or less stringent conditions may also be selected. Generally, low stringency conditions are selected to be about 50° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. For example, stringent conditions are those that are at least as stringent as, for example, conditions A-L; and reduced stringency conditions are at least as stringent as, for example, conditions M-R. Non-specific binding may be controlled using any one of a number of known techniques such as, for example, blocking the membrane with protein containing solutions, additions of heterologous RNA, DNA, and SDS to the hybridisation buffer, and treatment with Rnase.

Examples of hybridisation and wash conditions are listed in table 2:

TABLE 2

| | | | | Wash |
|---|---|---|---|---|
| Stringency | Polynucleotide | Hybrid Length | Hybridization Temperature | Temperature |
| Condition | Hybrid^{±} | (bp)^{‡} | and Buffer^{†} | and Buffer^{†} |
| A | DNA:DNA | > or | 65° C. 1xSSC; or 42° C., 1xSSC | 65° C.; 0.3xSSC |
| | | equal to 50 | and 50% formamide | |
| B | DNA:DNA | <50 | Tb*; 1xSSC | Tb*; 1xSSC |
| C | DNA:RNA | > or | 67° C. 1xSSC; or 45° C., 1xSSC | 67° C.; 0.3xSSC |
| | | equal to 50 | and 50% formamide | |
| D | DNA:RNA | <50 | Td*; 1xSSC | Td*; 1xSSC |
| E | RNA:RNA | > or | 70° C. 1xSSC; or 50° C., 1xSSC | 70° C.; 0.3xSSC |
| | | equal to 50 | and 50% formamide | |
| F | RNA:RNA | <50 | Tf*; 1xSSC | Tf*; 1xSSC |
| G | DNA:DNA | > or | 65° C. 4xSSC; or 45° C., 4xSSC | 65° C.; 1xSSC |
| | | equal to 50 | and 50% formamide | |
| H | DNA:DNA | <50 | Th*; 4 xSSC | Th*; 4xSSC |
| I | DNA:RNA | > or | 67° C. 4xSSC; or 45° C., 4xSSC | 67° C.; 1xSSC |
| | | equal to 50 | and 50% formamide | |
| J | DNA:RNA | <50 | Tj*; 4 xSSC | Tj*; 4 xSSC |
| K | RNA:RNA | > or | 70° C. 4xSSC; or 40° C., 6xSSC | 67° C.; 1xSSC |
| | | equal to 50 | and 50% formamide | |
| L | RNA:RNA | <50 | Tl*; 2 xSSC | Tl*; 2xSSC |
| M | DNA:DNA | > or | 50° C. 4xSSC; or 40° C., 6xSSC | 50° C.; 2xSSC |
| | | equal to 50 | and 50% formamide | |
| N | DNA:DNA | <50 | Tn*; 6 xSSC | Tn*; 6xSSC |
| O | DNA:RNA | > or | 55° C. 4xSSC; or 42° C., 6xSSC | 55° C.; 2xSSC |
| | | equal to 50 | and 50% formamide | |
| P | DNA:RNA | <50 | Tp*; 6 xSSC | Tp*; 6xSSC |
| Q | RNA:RNA | > or | 60° C. 4xSSC; or 45° C., 6xSSC | 60° C.; 2xSSC |
| | | equal to 50 | and 50% formamide | |
| R | RNA:RNA | <50 | Tr*; 4 xSSC | Tr*; 4xSSC |

^{‡}The “hybrid length” is the anticipated length for the hybridising nucleic acid. When nucleic acids of known sequence are hybridised, the hybrid length may be determined by aligning the sequences and identifying the conserved regions described herein.
^{†}SSPE (1xSSPE is 0.15M NaCl, 10 mM NaH₂PO₄, and 1.25 mM EDTA, pH 7.4) may be substituted for SSC (1xSSC is 0.15M NaCl and 15 mM sodium citrate) in the hybridisation and wash buffers; washes are performed for 15 minutes after hybridisation is complete. The hybridisations and washes may additionally include 5 × Denhardt's reagent, 0.5-1.0% SDS, 100 μg/ml denatured, fragmented salmon sperm DNA, 0.5% sodium pyrophosphate, and up to 50% formamide.
*Tb − Tr: The hybridisation temperature for hybrids anticipated to be less than 50 base pairs in length should be 5-10° C. less than the melting temperature Tₘ of the hybrids; the Tₘ is determined according to the above-mentioned equations.
^{±}The present invention also encompasses the substitution of any one, or more DNA or RNA hybrid partners with either a PNA, or a modified nucleic acid.

For the purposes of defining the level of stringency, reference can conveniently be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989).

For example, a nucleic acid encoding SEQ ID NO: 2 or a homologue thereof may be used in a hybridisation experiment. Alternatively fragments thereof may be used as probes. Depending on the starting pool of sequences from which the HKT protein is to be identified, different fragments for hybridization may be selected. For example, when a limited number of homologues with a high sequence identity to HKT are desired, a less conserved fragment may be used for hybridisation. By aligning SEQ ID NO: 2 and homologues thereof, it is possible to design equivalent nucleic acid fragments useful as probes for hybridisation.

After hybridisation and washing, the duplexes may be detected by autoradiography (where radiolabeled probes are used) or by chemiluminescence, immunodetection, by fluorescent or chromogenic detection, depending on the type of probe labelling. Alternatively, a ribonuclease protection assay may be performed for detection of RNA: RNA hybrids

The HKT nucleic acid or variant thereof may be derived from any natural or artificial source. The nucleic acid/gene or variant thereof may be isolated from a microbial source, such as bacteria, yeast or fungi, or from a plant, algae or animal (including human) source. This nucleic acid may be modified from its native form in composition and/or genomic environment through deliberate human manipulation. The nucleic acid is preferably of plant origin, whether from the same plant species (for example to the one in which it is to be introduced) or whether from a different plant species. The nucleic acid may be isolated from a dicotyledonous species, preferably from the family Brassicaceae, further preferably from Arabidopsis thaliana. More preferably, the HKT isolated from Arabidopsis thaliana is represented by SEQ ID NO: 1 and the HKT amino acid sequence is as represented by SEQ ID NO: 2.

The activity of an HKT polypeptide or a homologue thereof may be increased by introducing a genetic modification (preferably in the locus of an HKT gene). The locus of a gene as defined herein is taken to mean a genomic region which includes the gene of interest and 10 kB up- or downstream of the coding region.

The genetic modification may be introduced, for example, by any one (or more) of the following methods: TDNA activation, TILLING, site-directed mutagenesis, homologous recombination, directed evolution or by introducing and expressing in a plant a nucleic acid encoding an HKT polypeptide or a homologue thereof, provided that each of the methods requires human intervention. Following introduction of the genetic modification there follows a step of selecting for increased activity of an HKT polypeptide, which increase in activity gives plants having improved growth characteristics.

T-DNA activation tagging (Hayashi et al. Science (1992) 1350-1353) involves insertion of T-DNA usually containing a promoter (may also be a translation enhancer or an intron), in the genomic region of the gene of interest or 10 kB up- or downstream of the coding region of a gene in a configuration such that such promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to overexpression of genes near to the inserted T-DNA. The resulting transgenic plants show dominant phenotypes due to overexpression of genes close to the introduced promoter. The promoter to be introduced may be any promoter capable of directing expression of a gene in the desired organism, in this case a plant. For example, constitutive, tissue-specific, cell type-specific and inducible promoters are all suitable for use in T-DNA activation.

A genetic modification may also be introduced in the locus of an HKT gene using the technique of TILLING (Targeted Induced Local Lesions IN Genomes). This is a mutagenesis technology useful to generate and/or identify, and to eventually isolate mutagenised variants of an HKT nucleic acid capable of exhibiting HKT activity. TILLING also allows selection of plants carrying such mutant variants. These mutant variants may even exhibit higher HKT activity than that exhibited by the gene in its natural form. TILLING combines high-density mutagenesis with high-throughput screening methods. The steps typically followed in TILLING are: (a) EMS mutagenesis (Redei and Koncz (1992), In: C Koncz, N-H Chua, J Schell, eds, Methods in Arabidopsis Research. World Scientific, Singapore, pp 16-82; Feldmann et al., (1994) In: E M Meyerowitz, C R Somerville, eds, Arabidopsis. Cold Spring Harbor Laboratory Press, Cold Spring Harbor, N.Y., pp 137-172; Lightner and Caspar (1998), In: J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum, Nat Biotechnol. 2000 April; 18(4):455-7, Stemple, Nature Rev. Genet. 5, 145-150, 2004).

Site directed mutagenesis may be used to generated variants of HKT nucleic acids or portions thereof that retain HKT activity, for example cation transporter activity. Several methods are available to achieve site directed mutagenesis, the most common being PCR-based methods (See for example Ausubel et al., Current Protocols in Molecular Biology. Wiley Eds. http://www.4ulr.com/products/currentprotocols/index.html).

Directed evolution may be used to generate functional variants of HKT nucleic acid molecules encoding HKT polypeptides or homologues, or portions thereof having an increased biological activity as outlined above. Directed evolution consists of iterations of DNA shuffling followed by appropriate screening and/or selection (Castle et al., (2004) Science 304(5674): 1151-4; U.S. Pat. Nos. 5,811,238 and 6,395,547).

TDNA activation, TILLING, site-directed mutagenesis and directed evolution are examples of technologies that enable the generation novel alleles and variants of HKT that retain HKT function and which are therefore useful in the methods of the invention.

Homologous recombination allows introduction in a genome of a selected nucleic acid at a defined selected position. Homologous recombination is a standard technology used routinely in biological sciences for lower organism such as yeast or the moss Physcomitrella. Methods for performing homologous recombination in plants have been described not only for model plants (Offringa et al. (1990) EMBO J. 9, 3077-3084) but also for crop plants, for example rice (Terada et al., (2002) Nature Biotechnol. 20, 1030-1034; or lida and Terada (2004) Curr. Opin. Biotechnol. 15, 132-138). The nucleic acid to be targeted (which may be an HKT nucleic acid molecule or variant thereof as hereinbefore defined) need not be targeted to the locus of an HKT gene, but may be introduced in, for example, regions of high expression. The nucleic acid to be targeted may be an improved allele used to replace the endogenous gene or may be introduced in addition to the endogenous gene.

According to a preferred embodiment of the invention, plant growth characteristics may be improved by introducing and expressing in a plant an isolated nucleic acid encoding an HKT polypeptide or a homologue thereof.

A preferred method for introducing a genetic modification (which in this case need not be in the locus of an HKT gene) is to introduce and express in a plant a nucleic acid encoding an HKT polypeptide or a homologue thereof. An HKT polypeptide or a homologue thereof as mentioned above is a polypeptide having HKT activity and four units of a transmembrane domain—pore forming domain—transmembrane domain. The HKT polypeptide has, in increasing order of preference, at least 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95% sequence identity to the amino acid sequence represented by SEQ ID NO: 2, or is as represented in SEQ ID NO: 2.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

Also encompassed by the term “homologues” are two special forms of homology, which include orthologous sequences and paralogous sequences, which encompass evolutionary concepts used to describe ancestral relationships of genes. The term “paralogous” relates to gene-duplications within the genome of a species leading to paralogous genes. The term “orthologous” relates to homologous genes in different organisms due to speciation.

Orthologues in, for example, monocot plant species may easily be found by performing a so-called reciprocal blast search. This may be done by a first blast involving blasting the sequence in question (for example, SEQ ID NO 1 or SEQ ID NO 2, being from Arabidopsis thaliana) against any sequence database, such as the publicly available NCBI database which may be found at: http://www.ncbi.nim.nih.gov. If orthologues in rice were sought, the sequence in question would be blasted against, for example, the 28,469 full-length cDNA clones from Oryza sativa Nipponbare available at NCBI. BLASTn or tBLASTX may be used when starting from nucleotides or BLASTP or TBLASTN when starting from the protein, with standard default values. The blast results may be filtered. The full-length sequences of either the filtered results or the non-filtered results are then blasted back (second blast) against the sequences of the organism from which the sequence in question is derived, in casu Arabidopsis thaliana. The results of the first and second blasts are then compared. An orthologue is found when the results of the second blast give as hits with the highest similarity a query HKT nucleic acid or HKT polypeptide. If one of the hits in the first BLAST is from the same organism, then a paralogue has been found. Such paralogue is also considered a homologue of HKT, provided that this homologue has HKT activity and comprises four units of a transmembrane domain—pore forming domain—transmembrane domain and preferably also comprises the conserved sequences defined above. In the case of large families, ClustalW may be used, followed by the construction of a neighbour joining tree, to help visualize the clustering.

A homologue may be in the form of a “substitutional variant” of a protein, i.e. where at least one residue in an amino acid sequence has been removed and a different residue inserted in its place. Amino acid substitutions are typically of single residues, but may be clustered depending upon functional constraints placed upon the polypeptide; insertions will usually be of the order of about 1 to 10 amino acid residues. Preferably, amino acid substitutions comprise conservative amino acid substitutions (Table 3). To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W.H. Freeman and Company).

TABLE 3
Examples of conserved amino acid substitutions:

| | Conservative | | Conservative |
|---|---|---|---|
| Residue | Substitutions | Residue | Substitutions |
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu, Val | | |

Less conserved substitutions may be made in case the above-mentioned amino acid properties are not so critical.

A homologue may also be in the form of an “insertional variant” of a protein, i.e. where one or more amino acid residues are introduced into a predetermined site in a protein. Insertions may comprise amino-terminal and/or carboxy-terminal fusions as well as intra-sequence insertions of single or multiple amino acids. Generally, insertions within the amino acid sequence will be smaller than amino- or carboxy-terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine)6-tag, glutathione S-transferase-tag, protein A, maltose-binding protein, dihydrofolate reductase, Tag 100 epitope, c-myc epitope, FLAG®-epitope, lacZ, CMP (calmodulin-binding peptide), HA epitope, protein C epitope and VSV epitope.

Homologues in the form of “deletion variants” of a protein are characterised by the removal of one or more amino acids from a protein.

Amino acid variants of a protein may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

The HKT polypeptide or homologue thereof may be a derivative. “Derivatives” include peptides, oligopeptides, polypeptides, proteins and enzymes which may comprise substitutions, deletions or additions of naturally and non-naturally occurring amino acid residues compared to the amino acid sequence of a naturally-occurring form of the protein, for example, as presented in SEQ ID NO 2. “Derivatives” of a protein encompass peptides, oligopeptides, polypeptides, proteins and enzymes which may comprise naturally occurring altered, glycosylated, acylated or non-naturally occurring amino acid residues compared to the amino acid sequence of a naturally-occurring form of the polypeptide. A derivative may also comprise one or more non-amino acid substituents compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

The HKT polypeptide or homologue thereof may be encoded by an alternative splice variant of an HKT nucleic acid molecule or gene. The term “alternative splice variant” as used herein encompasses variants of a nucleic acid sequence in which selected introns and/or exons have been excised, replaced or added. Such variants will be ones in which the biological activity of the protein is retained, which may be achieved by selectively retaining functional segments of the protein. Such splice variants may be found in nature or may be manmade. Methods for making such splice variants are well known in the art. Preferred splice variants are splice variants derived from the nucleic acid represented by SEQ ID NO: 3. Further preferred are splice variants encoding a polypeptide having HKT activity and comprising at least one, and preferably four unit(s) of a transmembrane domain—pore forming domain—transmembrane domain. A preferred splice variant is represented by SEQ ID NO: 1.

The homologue may also be encoded by an allelic variant of a nucleic acid encoding an HKT polypeptide or a homologue thereof, preferably an allelic variant of the nucleic acid represented by SEQ ID NO 1. Further preferably, the polypeptide encoded by the allelic variant has HKT activity and comprises at least one, and preferably four unit(s) of a transmembrane domain—pore forming domain—transmembrane domain. The homologue may also be encoded by an allelic variant of a nucleic acid represented by SEQ ID NO: 11, 13, 15, 17, 19, 21, 23, 25, 27, 29, 35 or 37. Allelic variants exist in nature and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

According to a preferred aspect of the present invention, enhanced or increased expression of the HKT nucleic acid molecule or variant thereof is envisaged. Methods for obtaining enhanced or increased expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of an HKT nucleic acid or variant thereof. For example, endogenous promoters may be altered in vivo by mutation, deletion, and/or substitution (see Kmiec, U.S. Pat. No. 5,565,350; Zarling et al., PCT/US93/03868), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, Mol. Cell Biol. 8, 4395-4405 (1988); Callis et al., Genes Dev. 1, 1183-1200 (1987)). Such intron enhancement of gene expression is typically greatest when placed near the 5′ end of the transcription unit. Use of the maize introns Adh1-S intron 1, 2, and 6, the Bronze-1 intron are known in the art. See generally, The Maize Handbook, Chapter 116, Freeling and Walbot, Eds., Springer, N.Y. (1994).

The invention also provides genetic constructs and vectors to facilitate introduction and/or expression of the nucleotide sequences useful in the methods according to the invention.

Therefore, there is provided a gene construct comprising:
(i) an isolated HKT nucleic acid molecule or functional variant thereof;
(ii) one or more control sequence(s) capable of driving expression of the nucleic acid sequence of (i); and optionally
(iii) a transcription termination sequence.

Preferably, the control sequence used in the gene construct is a seed-specific control sequence.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells.

Plants are transformed with a vector comprising the sequence of interest (i.e., an HKT nucleic acid or variant thereof). The sequence of interest is operably linked to one or more control sequences (at least to a promoter). The terms “regulatory element”, “control sequence” and “promoter” are all used interchangeably herein and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli (in case of an inducible promoter), or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative which confers, activates or enhances expression of a nucleic acid molecule in a cell, tissue or organ. The term “operably linked” as used herein refers to a functional linkage between the promoter sequence and the gene of interest, such that the promoter sequence is able to initiate transcription of the gene of interest.

Advantageously, any type of promoter may be used to drive expression of the nucleic acid sequence. The promoter may be an inducible promoter, i.e. having induced or increased transcription initiation in response to a developmental, chemical, environmental or physical stimulus. An example of such a promoter is a stress-inducible promoter. Additionally or alternatively, the promoter may be a constitutive promoter. The term “constitutive” as defined herein refers to a promoter that is expressed predominantly in at least one tissue or organ and predominantly at any life stage of the plant. Preferably the constitutive promoter is expressed predominantly throughout the plant. Additionally or alternatively, the promoter may be a tissue-specific promoter, i.e. one that is capable of preferentially initiating transcription in certain tissues, such as the leaves, roots, seed tissue etc.

Preferably, the isolated HKT nucleic acid or variant thereof is operably linked to a seed-specific promoter. Further preferably, the seed-specific promoter is embryo- and aleurone-specific and mainly active during the late embryogenic stages. The term “seed-specific” as defined herein refers to a promoter that is expressed predominantly in the seeds of the plant. Preferably the promoter is expressed predominantly in the embryo and/or aleurone layer. More preferably, the seed-specific promoter has a comparable expression profile to the WSI18 promoter. Most preferably, the seed-specific promoter is the WSI18 promoter from rice (WO2004/070039), as given in SEQ ID NO: 45 (corresponding to nucleotides 1 to 1828 of SEQ ID NO: 4). The WSI18 promoter from rice is responsive to abscisic acid (ABA) and highly induced upon conditions that involve ABA, such as seed desiccation. Therefore, a preferred promoter for use in the present invention may also be any other promoter induced by ABA and/or by stress conditions such as drought. It should be clear however that the applicability of the present invention is not restricted to the HKT nucleic acid represented by SEQ ID NO: 1, nor is the applicability of the invention restricted to expression of an HKT nucleic acid when driven by a WSI18 promoter. Examples of other seed-specific promoters that may also be used to drive expression of an HKT nucleic acid are listed in Table 4.

TABLE 4
Examples of seed-specific promoters for use in the performance of the present invention:

| | EXPRESSION | |
|---|---|---|
| GENE SOURCE | PATTERN | REFERENCE |
| seed-specific genes | seed | Simon, et al., Plant Mol. Biol. 5: 191, |
| | | 1985; Scofield, et al., J. Biol. Chem. |
| | | 262: 12202, 1987.; Baszczynski, et al., |
| | | Plant Mol. Biol. 14: 633, 1990. |
| Brazil Nut albumin | seed | Pearson, et al., Plant Mol. Biol. 18: 235-245, |
| | | 1992. |
| legumin | seed | Ellis, et al., Plant Mol. Biol. 10: 203-214, |
| | | 1988. |
| glutelin (rice) | seed | Takaiwa, et al., Mol. Gen. Genet. 208: |
| | | 15-22, 1986; Takaiwa, et al., FEBS |
| | | Letts. 221: 43-47, 1987. |
| zein | seed | Matzke et al Plant Mol Biol, 14(3): 323-32 |
| | | 1990 |
| napA | seed | Stalberg, et al, Planta 199: 515-519, |
| | | 1996. |
| wheat LMW and HMW | endosperm | Mol Gen Genet 216: 81-90, 1989; NAR |
| glutenin-1 | | 17: 461-2, 1989 |
| wheat SPA | seed | Albani et al, Plant Cell, 9: 171-184, 1997 |
| wheat α, β, γ-gliadins | endosperm | EMBO J. 3: 1409-15, 1984 |
| barley Itr1 promoter | endosperm | |
| barley B1, C, D, hordein | endosperm | Theor Appl Gen 98: 1253-62, 1999; |
| | | Plant J 4: 343-55, 1993; Mol Gen Genet |
| | | 250: 750-60, 1996 |
| barley DOF | endosperm | Mena et al, The Plant Journal, 116(1): |
| | | 53-62, 1998 |
| blz2 | endosperm | EP99106056.7 |
| synthetic promoter | endosperm | Vicente-Carbajosa et al., Plant J. 13: |
| | | 629-640, 1998. |
| rice prolamin NRP33 | endosperm | Wu et al, Plant Cell Physiology 39(8) |
| | | 885-889, 1998 |
| rice α-globulin Glb-1 | endosperm | Wu et al, Plant Cell Physiology 39(8) |
| | | 885-889, 1998 |
| rice OSH1 | embryo | Sato et al, Proc. Natl. Acad. Sci. USA, |
| | | 93: 8117-8122, 1996 |
| rice α-globulin REB/OHP-1 | endosperm | Nakase et al. Plant Mol. Biol. 33: 513-522, |
| | | 1997 |
| rice ADP-glucose PP | endosperm | Trans Res 6: 157-68, 1997 |
| maize ESR gene family | endosperm | Plant J 12: 235-46, 1997 |
| sorgum γ-kafirin | endosperm | PMB 32: 1029-35, 1996 |
| KNOX | embryo | Postma-Haarsma et al, Plant Mol. Biol. |
| | | 39: 257-71, 1999 |
| rice oleosin | embryo and aleuron | Wu et at, J. Biochem., 123: 386, 1998 |
| sunflower oleosin | seed (embryo and dry | Cummins, et al., Plant Mol. Biol. 19: |
| | seed) | 873-876, 1992 |
| PRO0117, putative rice 40S | weak in endosperm | WO2004/070039 |
| ribosomal protein | | |
| PRO0135, rice alpha-globulin | strong in endosperm | |
| PRO0136, rice alanine | weak in endosperm | |
| aminotransferase | | |
| PRO0147, trypsin inhibitor | weak in endosperm | |
| ITR1 (barley) | | |
| PRO0175, rice RAB21 | embryo + stress | WO2004/070039 |
| PRO0218, rice oleosin 18 kd | aleurone + embryo | |

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences which may be suitable for use in performing the invention. Such sequences would be known or may readily be obtained by a person skilled in the art.

The genetic constructs of the invention may further include an origin of replication sequence, which is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

The genetic construct may optionally comprise a selectable marker gene. As used herein, the term “selectable marker gene” includes any gene which confers a phenotype on a cell in which it is expressed to facilitate the identification and/or selection of cells which are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin), to herbicides (for example bar which provides resistance to Basta; aroA or gox providing resistance against glyphosate), or genes that provide a metabolic trait (such as manA that allows plants to use mannose as sole carbon source). Visual marker genes result in the formation of colour (for example β-glucuronidase, GUS), luminescence (such as luciferase) or fluorescence (Green Fluorescent Protein, GFP, and derivatives thereof).

In a preferred embodiment, the genetic construct as mentioned above, comprises an HKT nucleic acid in sense orientation coupled to a promoter that is preferably a seed-specific promoter, such as for example the rice WSI18 promoter. Therefore, another aspect of the present invention is a vector construct carrying an expression cassette essentially similar to SEQ ID NO 4, comprising a WSI18 promoter, the rice HKT gene and the T-zein+T-rubisco deltaGA transcription terminator sequence. A sequence essentially similar to SEQ ID NO 4 encompasses a first nucleic acid sequence encoding a protein homologous to SEQ ID NO 2 or hybridising to SEQ ID NO 1, which first nucleic acid is operably linked to a WSI18 promoter or a promoter with a similar expression pattern, additionally or alternatively the first nucleic acid is linked to a transcription termination sequence.

The present invention also encompasses plants or parts (including plant cells) thereof obtainable by the methods according to the present invention. The present invention therefore provides plants or parts thereof (including plant cells) obtainable by the method according to the present invention, which plants have introduced therein an isolated HKT nucleic acid or variant thereof, or which plants have introduced therein a genetic modification, preferably in the locus of an HKT gene.

The invention also provides a method for the production of transgenic plants having improved growth characteristics, comprising introduction and expression in a plant of an isolated HKT nucleic acid or a variant thereof.

More specifically, the present invention provides a method for the production of transgenic plants having improved growth characteristics, which method comprises:
(i) introducing into a plant or plant cell an isolated HKT nucleic acid or variant thereof; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation.

The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation of plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens et al. (1982) Nature 296, 72-74; Negrutiu et al. (1987) Plant Mol. Biol. 8, 363-373); electroporation of protoplasts (Shillito et al. (1985) Bio/Technol 3, 1099-1102); microinjection into plant material (Crossway et al. (1986) Mol. Gen. Genet. 202, 179-185); DNA or RNA-coated particle bombardment (Klein et al. (1987) Nature 327, 70) infection with (non-integrative) viruses and the like. Transgenic rice plants expressing an HKT protein are preferably produced via Agrobacterium-mediated transformation using any of the well known methods for rice transformation, such as described in any of the following: published European patent application EP 1198985 A1, Aldemita and Hodges (Planta 199, 612-617, 1996); Chan et al. (Plant Mol. Biol. 22, 491-506, 1993), Hiei et al. (Plant J. 6, 271-282, 1994), which disclosures are incorporated by reference herein as if fully set forth. In the case of corn transformation, the preferred method is as described in either Ishida et al. (Nature Biotechnol. 14, 745-50, 1996) or Frame et al. (Plant Physiol. 129, 13-22, 2002), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art. The cultivation of transformed plant cells into mature plants may thus encompass steps of selection and/or regeneration and/or growing to maturity.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. The invention also includes host cells containing an isolated HKT nucleic acid or variant thereof. Preferred host cells according to the invention are plant cells. The invention also extends to harvestable parts of a plant according to the invention, such as but not limited to seeds, leaves, fruits, flowers, stems, rhizomes, tubers and bulbs. The invention furthermore relates to products directly derived from a harvestable part of such a plant, such as dry pellets or powders, oil, fat and fatty acids, starch or proteins.

The present invention also encompasses the use of HKT nucleic acids or variants thereof and to the use of HKT polypeptides or homologues thereof.

One such use relates to improving the growth characteristics of plants, in particular in improving yield, especially seed yield. The seed yield may include one or more of the following: increased number of (filled) seeds, increased seed weight, increased harvest index, increased thousand kernel weight, seed filling rate, among others.

HKT nucleic acids or variants thereof or HKT polypeptides or homologues thereof may find use in breeding programmes in which a DNA marker is identified which may be genetically linked to an HKT gene or variant thereof. The HKT or variants thereof or HKT or homologues thereof may be used to define a molecular marker. This DNA or protein marker may then be used in breeding programmes to select plants having improved growth characteristics. The HKT gene or variant thereof may, for example, be a nucleic acid as represented by SEQ ID NO: 1, or a nucleic acid encoding any of the above-mentioned homologues.

Allelic variants of an HKT may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place by, for example, PCR. This is followed by a selection step for selection of superior allelic variants of the sequence in question and which give rise to improved growth characteristics in a plant. Selection is typically carried out by monitoring growth performance of plants containing different allelic variants of the sequence in question, for example, different allelic variants of SEQ ID NO: 1, or of nucleic acids encoding any of the above mentioned plant homologues. Growth performance may be monitored in a greenhouse or in the field. Further optional steps include crossing plants, in which the superior allelic variant was identified, with another plant. This could be used, for example, to make a combination of interesting phenotypic features.

HKT nucleic acids or variants thereof may also be used as probes for genetically and physically mapping the genes that they are a part of, and as markers for traits linked to those genes. Such information may be useful in plant breeding in order to develop lines with desired phenotypes. Such use of HKT nucleic acids or variants thereof requires only a nucleic acid sequence of at least 10 nucleotides in length. The HKT nucleic acids or variants thereof may be used as restriction fragment length polymorphism (RFLP) markers. Southern blots of restriction-digested plant genomic DNA may be probed with the HKT nucleic acids or variants thereof. The resulting banding patterns may then be subjected to genetic analyses using computer programs such as MapMaker (Lander et al. (1987) Genomics 1, 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the HKT nucleic acid or variant thereof in the genetic map previously obtained using this population (Botstein et al. (1980) Am. J. Hum. Genet. 32, 314-331).

The production and use of plant gene-derived probes for use in genetic mapping is described in Bematzky and Tanksley (Plant Mol. Biol. Reporter 4, 37-41, 1986). Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Nonmammalian Genomic Analysis: A Practical Guide, Academic press 1996, pp. 319-346, and references cited therein).

In another embodiment, the nucleic acid probes may be used in direct fluorescence in situ hybridization (FISH) mapping (Trask (1991) Trends Genet. 7, 149-154). Although current methods of FISH mapping favour use of large clones (several to several hundred kb; see Laan et al. (1995) Genome Res. 5, 13-20), improvements in sensitivity may allow performance of FISH mapping using shorter probes.

A variety of nucleic acid amplification-based methods of genetic and physical mapping may be carried out using the nucleic acids. Examples include allele-specific amplification (Kazazian (1989) J. Lab. Clin. Med. 11, 95-96), polymorphism of PCR-amplified fragments (CAPS; Sheffield et al. (1993) Genomics 16, 325-332), allele-specific ligation (Landegren et al. (1988) Science 241, 1077-1080), nucleotide extension reactions (Sokolov (1990) Nucleic Acid Res. 18, 3671), Radiation Hybrid Mapping (Walter et al. (1997) Nat. Genet. 7, 22-28) and Happy Mapping (Dear and Cook (1989) Nucleic Acid Res. 17, 6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

In this way, generation, identification and/or isolation of plants with increased HKT activity displaying improved growth characteristics can be performed.

HKT nucleic acids or variants thereof or HKT polypeptides or homologues thereof may also find use as growth regulators. Since these molecules have been shown to be useful in improving the growth characteristics of plants, they would also be useful growth regulators, such as herbicides or growth stimulators. The present invention therefore provides a composition comprising an HKT or variant thereof or an HKT polypeptide or homologue thereof, together with a suitable carrier, diluent or excipient, for use as a growth regulator, preferably as a growth promoter.

Performance of the methods according to the present invention result in plants having improved growth characteristics, as described hereinbefore. These advantageous growth characteristics may also be combined with other economically advantageous traits, such as further yield-enhancing traits, tolerance to various stresses, traits modifying various architectural features and/or biochemical and/or physiological features, with the proviso that the sequences represented in GenBank Acc nr U16709 are not used for modifying salt tolerance of a plant or for accumulating alkali metals.

### DESCRIPTION OF FIGURES

The present invention will now be described with reference to the following figures in which:

FIG. 1 Schematic representation of an HKT protein (Mäser et al., 2002). The transmembrane domains are labelled with roman numerals I to VIII and the four pore forming domains are indicated with the letters A to D. The alignment shows the pore forming domain A of various plant HKTs compared with Trk1 from S. cerevisiae (M21328), TrkH from Pseudomonas aeruginosa (AAG06598), KtrB from Vibrio alginolyticus (BAA32063), and to the pore forming domain of the Drosophila Shaker channel (S00479). The residue corresponding to the first glycine of the K+ channel GYG motif is marked with an asterisk.

FIG. 2 Multiple alignment of various HKT protein sequences. Database accession numbers are given, abbreviations used: At, Arabidopsis thaliana; Mc, Mesembryanthemum crystallinum; Ec, Eucalyptus camaldulensis; Os, Oryza sativa. JU0466 represents the TRK1 sequence from Saccharomyces uvarum and CAA82128 is the TRK2 sequence from Saccharomyces cerevisiae.

FIG. 3 Schematic presentation of the entry clone p036, containing CDS1532 within the AttL1 and AttL2 sites from Gateway® cloning in the pDONR201 backbone. CDS1532 is the internal code for the Arabidopsis HKT encoding sequence. This vector contains also a bacterial kanamycin-resistance cassette and a bacterial origin of replication.

FIG. 4 shows a binary vector for expression in Oryza sativa of the Arabidopsis HKT gene (internal reference CDS1532) under the control of the rice WSI18 promoter (internal reference PRO0151). This vector contains T-DNA derived from the Ti Plasmid, limited by a left border (LB repeat, LB Ti C58) and a right border (RB repeat, RB Ti C58)). From the left border to the right border, this T-DNA contains: a cassette for antibiotic selection of transformed plants; a constitutive promoter—selectable marker—NOS terminator cassette for visual screening of transformed plants; the PRO0151-CDS1532 construct-zein and rbcS-deltaGA double terminator cassette for expression of the Arabidopsis HKT gene. This vector also contains an origin of replication from pBR322 for bacterial replication and a selectable marker (Spe/SmeR) for bacterial selection with spectinomycin and streptomycin.

FIG. 5 details examples of sequences useful in performing the methods according to the present invention.

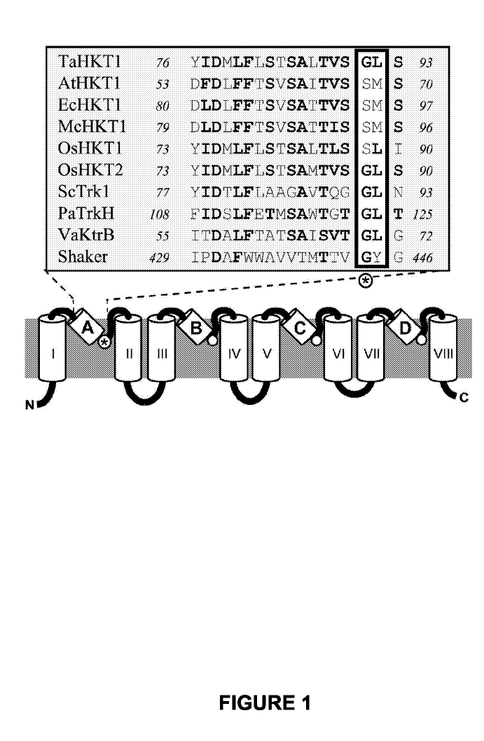

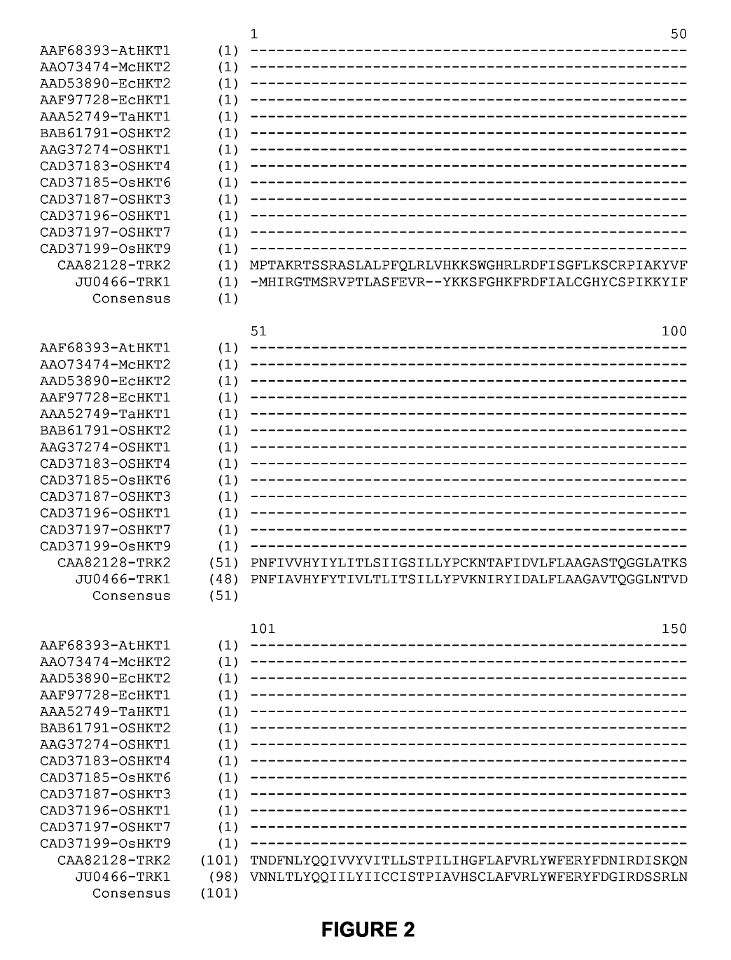

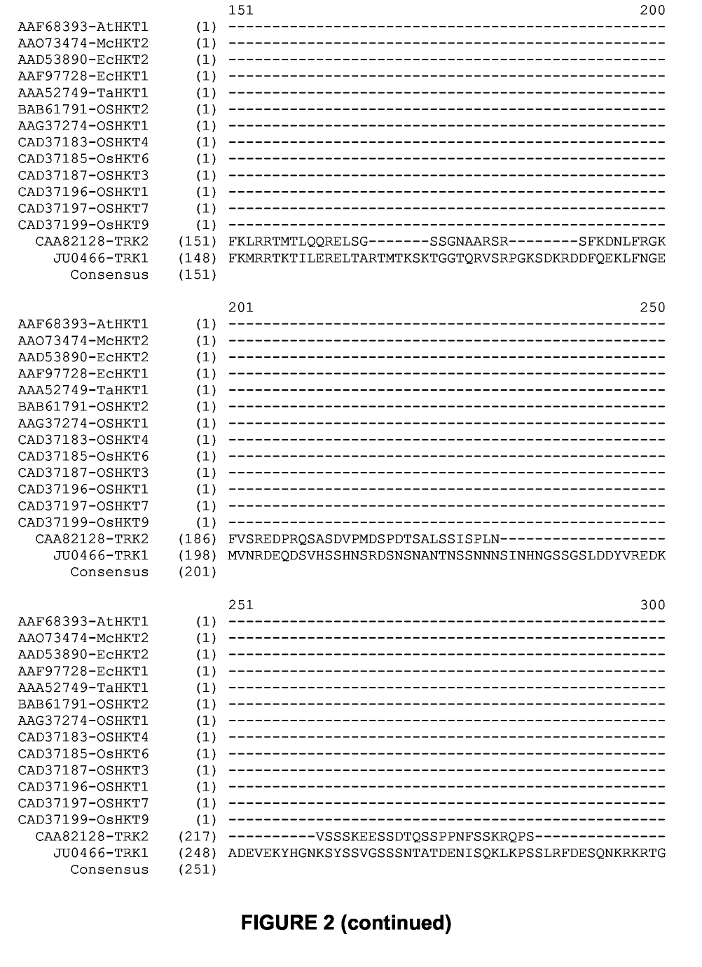

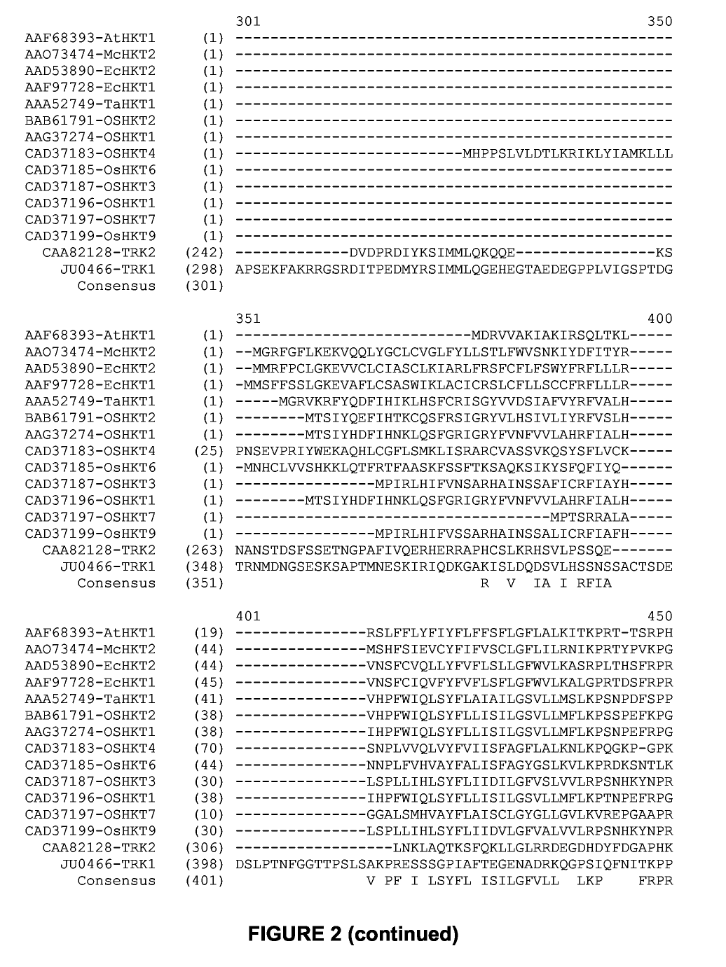

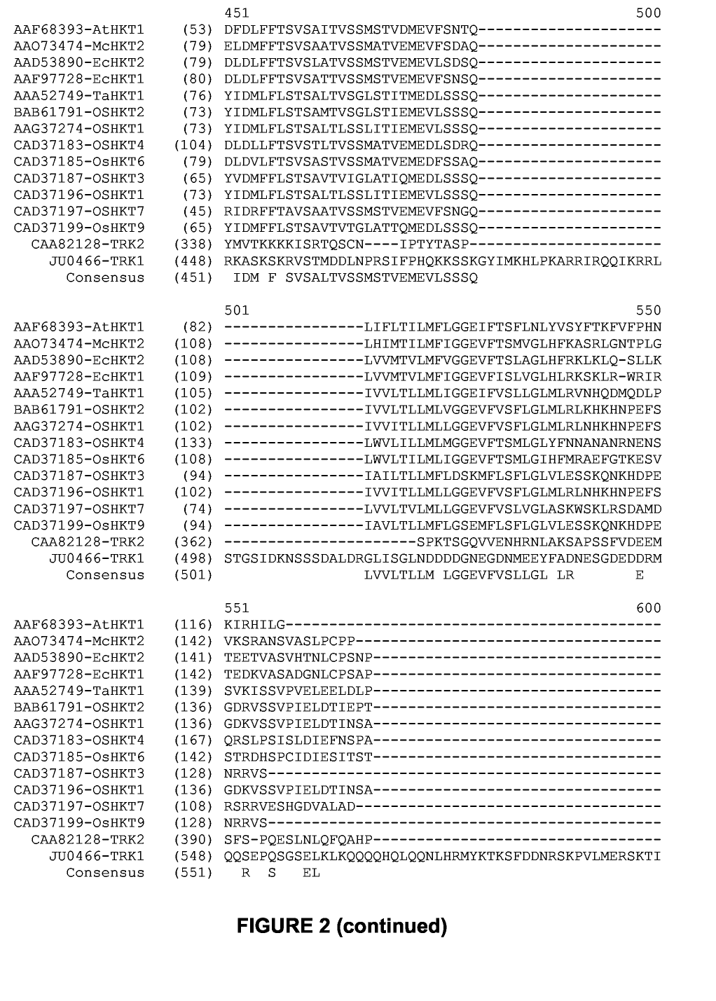

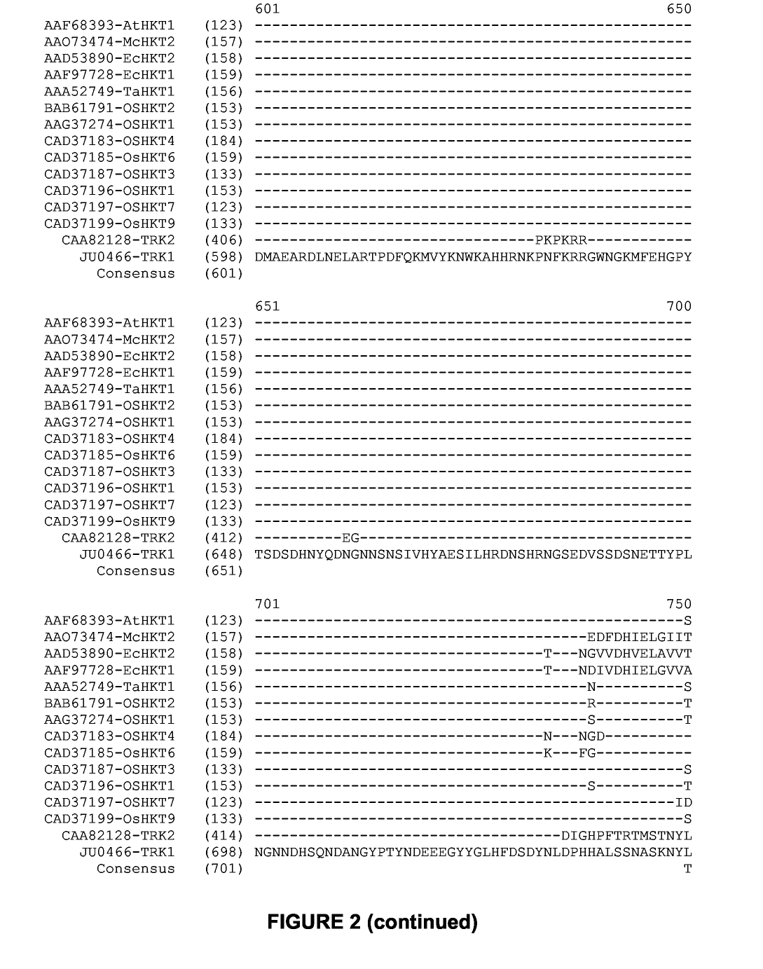

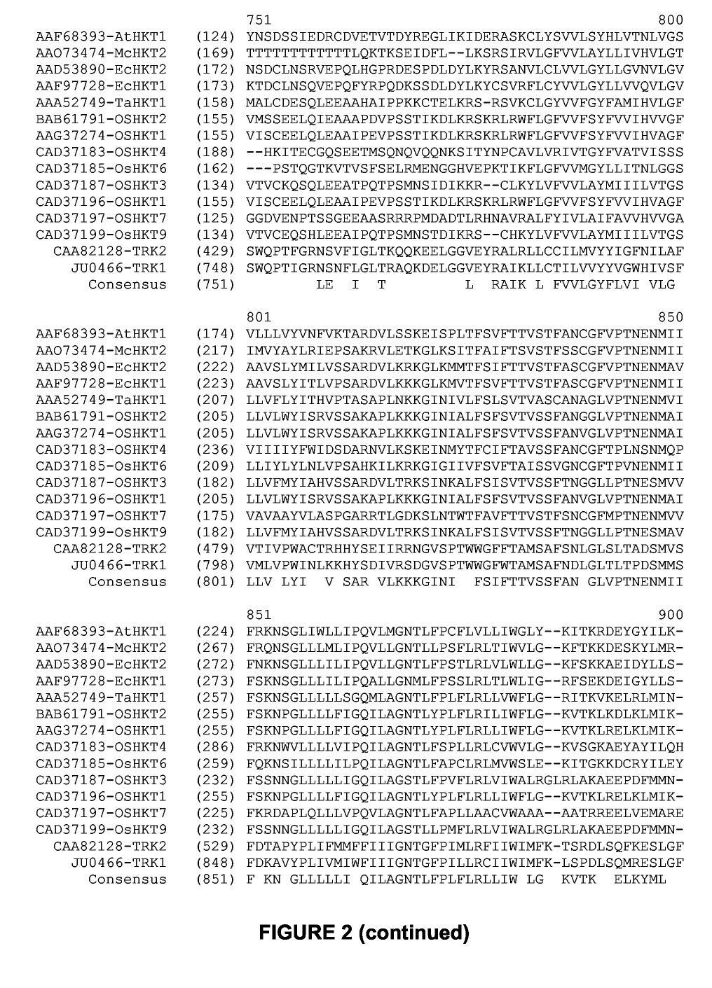

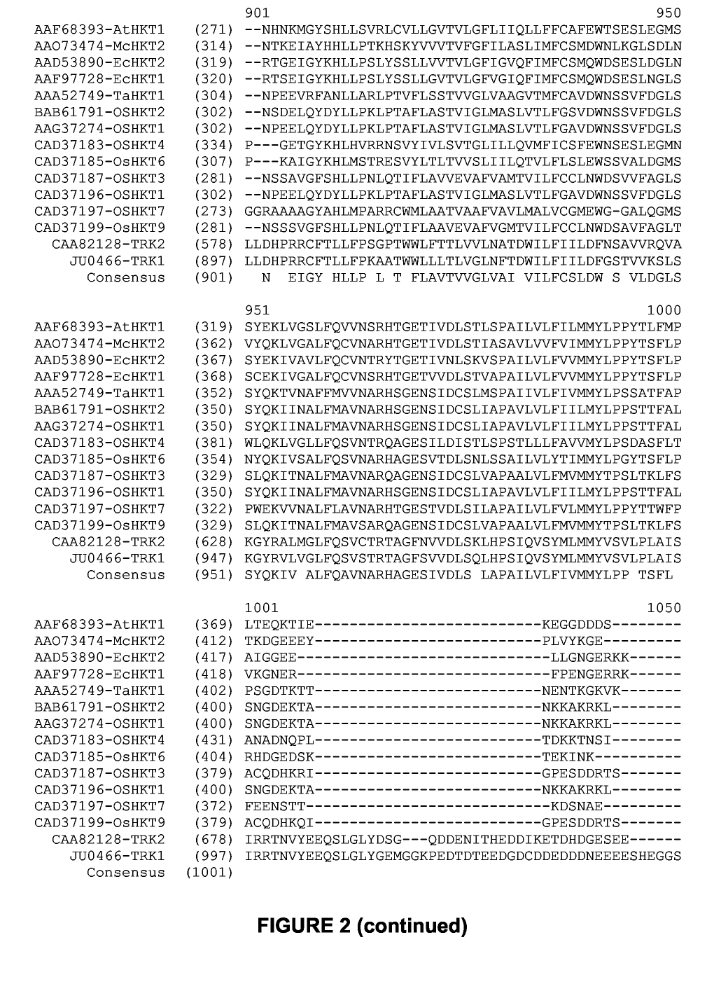

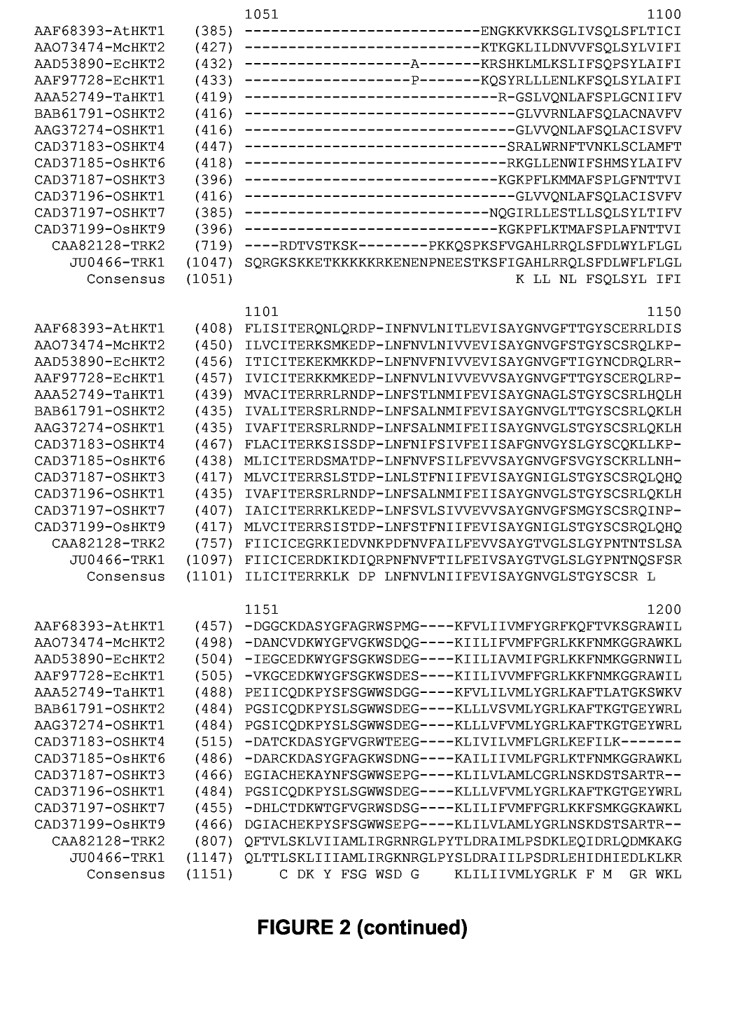

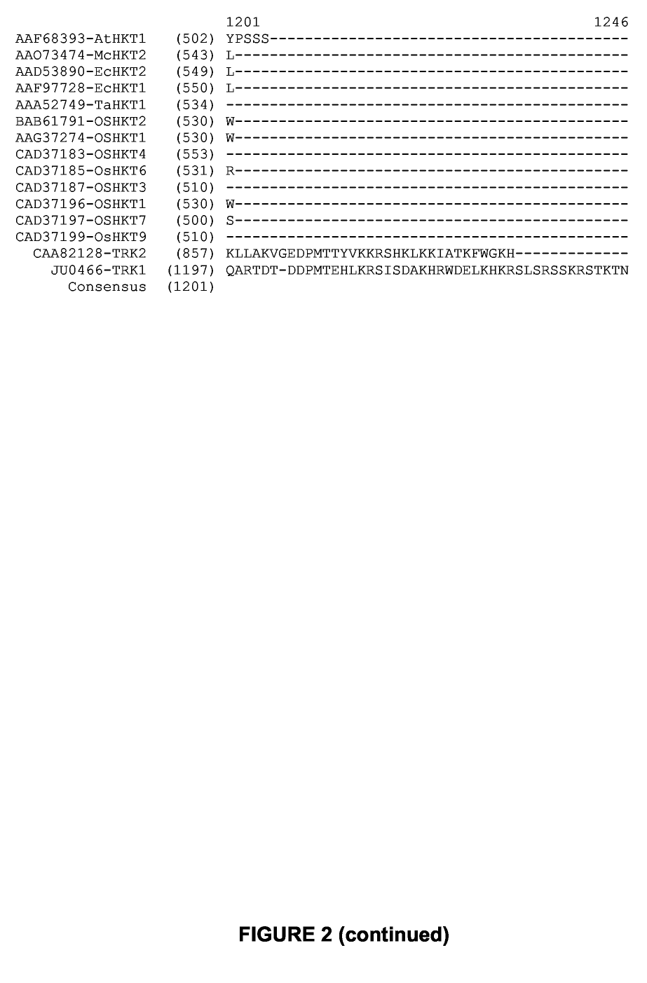

### EXAMPLES

The present invention will now be described with reference to the following examples, which are by way of illustration alone.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Gene Cloning

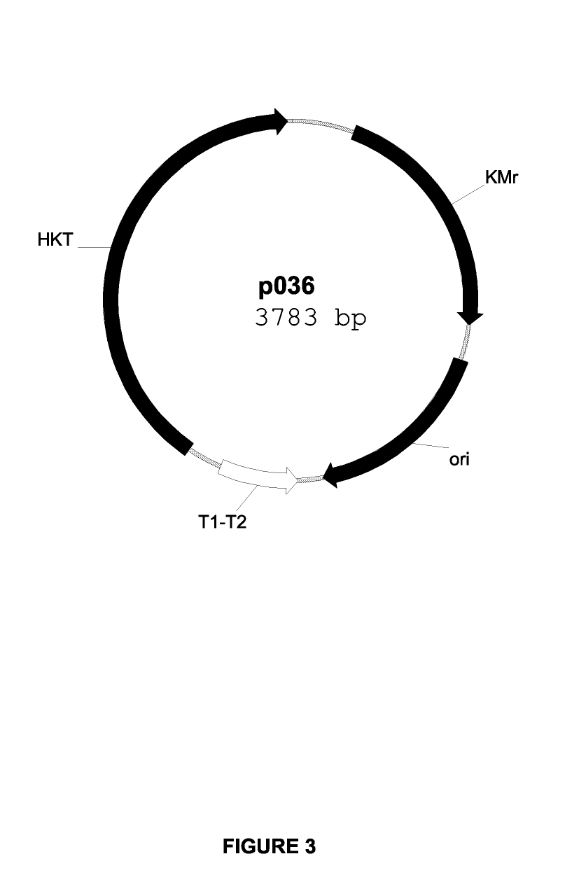

The Arabidopsis HKT (internal reference CDS1532) was amplified by PCR using as template an Arabidopsis thaliana seedling cDNA library (Invitrogen, Paisley, UK). After reverse transcription of RNA extracted from seedlings, the cDNAs were cloned into pCMV Sport 6.0. Average insert size of the bank was 1.5 kb, and original number of clones was of 1.59×10⁷ cfu. The original titer was determined to be 9.6×10⁵ cfu/ml, and became after a first amplification 6×10¹¹ cfu/ml. After plasmid extraction, 200 ng of template was used in a 50 μl PCR mix. Primers prm3283 (SEQ ID NO: 5) and prm8443 (SEQ ID NO: 6), which include the AttB sites for Gateway recombination, were used for PCR amplification. PCR was performed using Hifi Taq DNA polymerase under standard conditions. A PCR fragment of 1613 bp was amplified and purified also using standard methods. The first step of the Gateway procedure, the BP reaction, was then performed, during which the PCR fragment recombines in vivo with the pDONR201 plasmid to produce, according to the Gateway terminology, an “entry clone”, p036 (FIG. 3). Plasmid pDONR201 was purchased from Invitrogen, as part of the Gateway® technology.

### Example 2

### Vector Construction

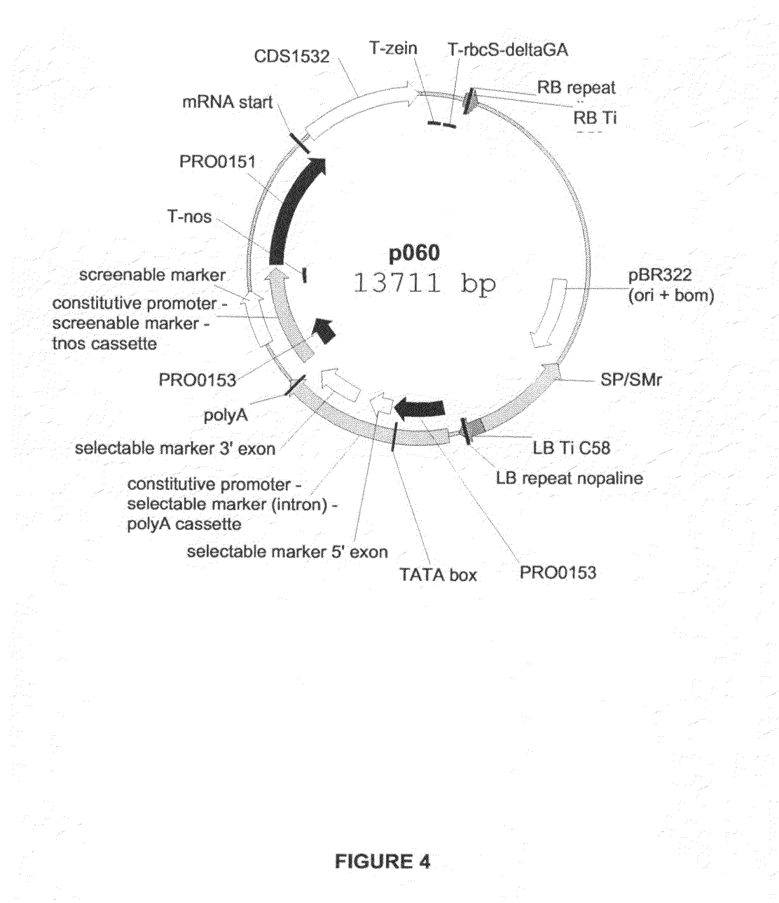

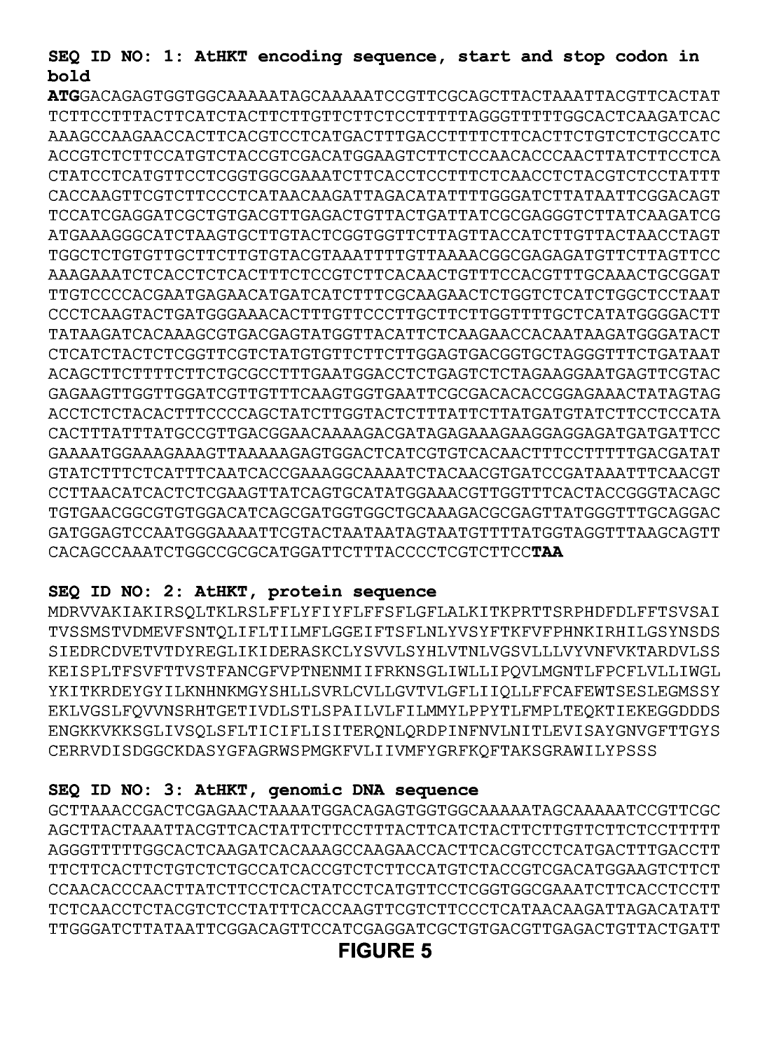

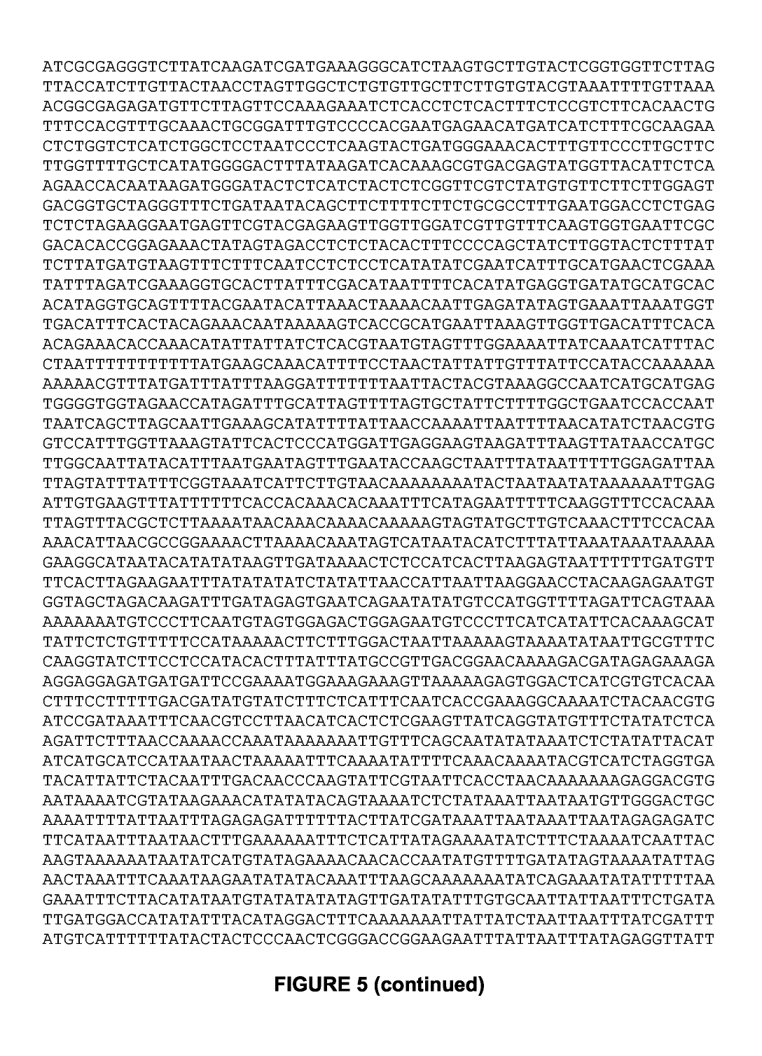

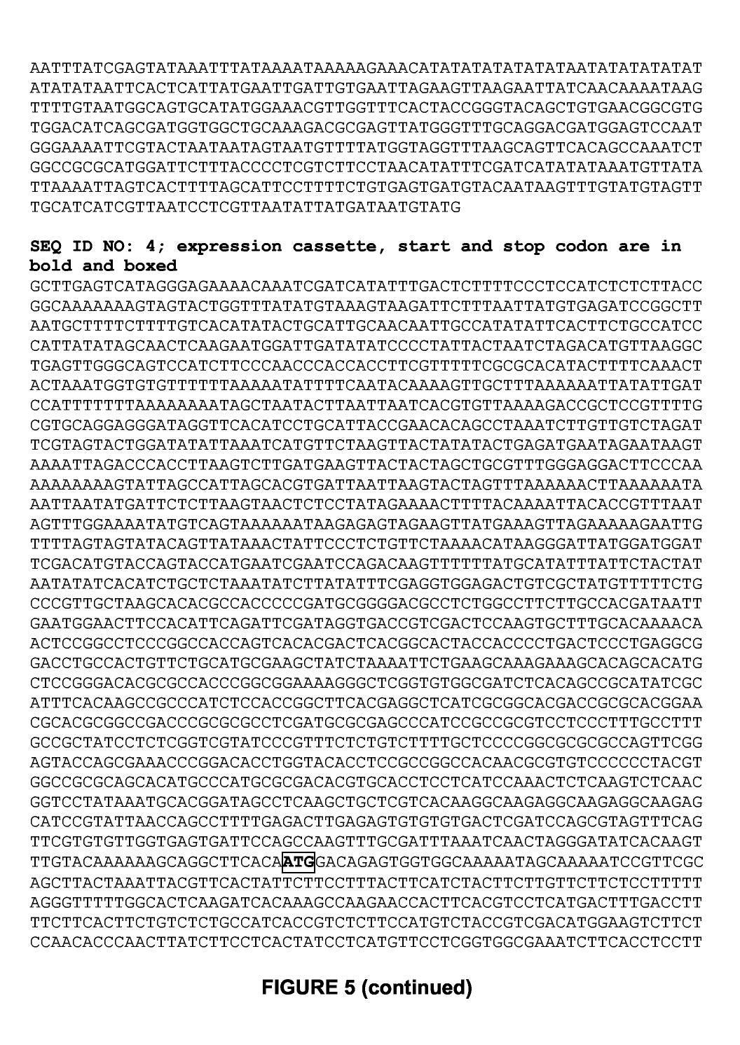

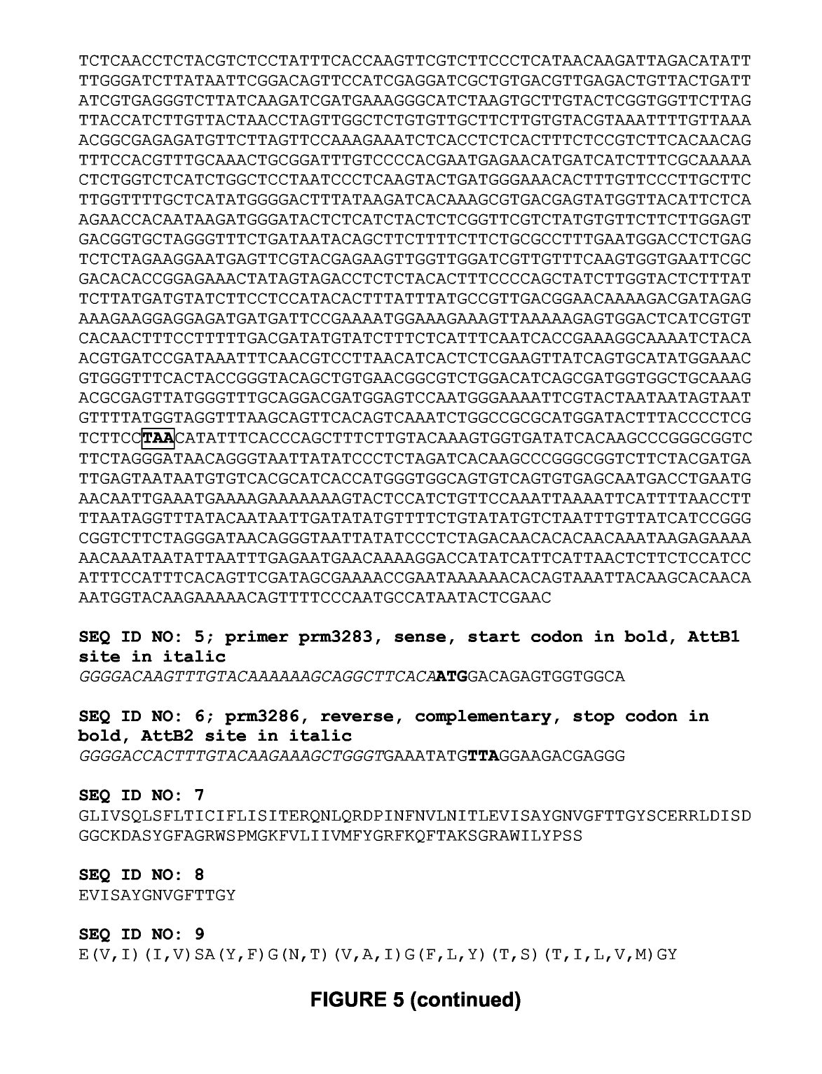

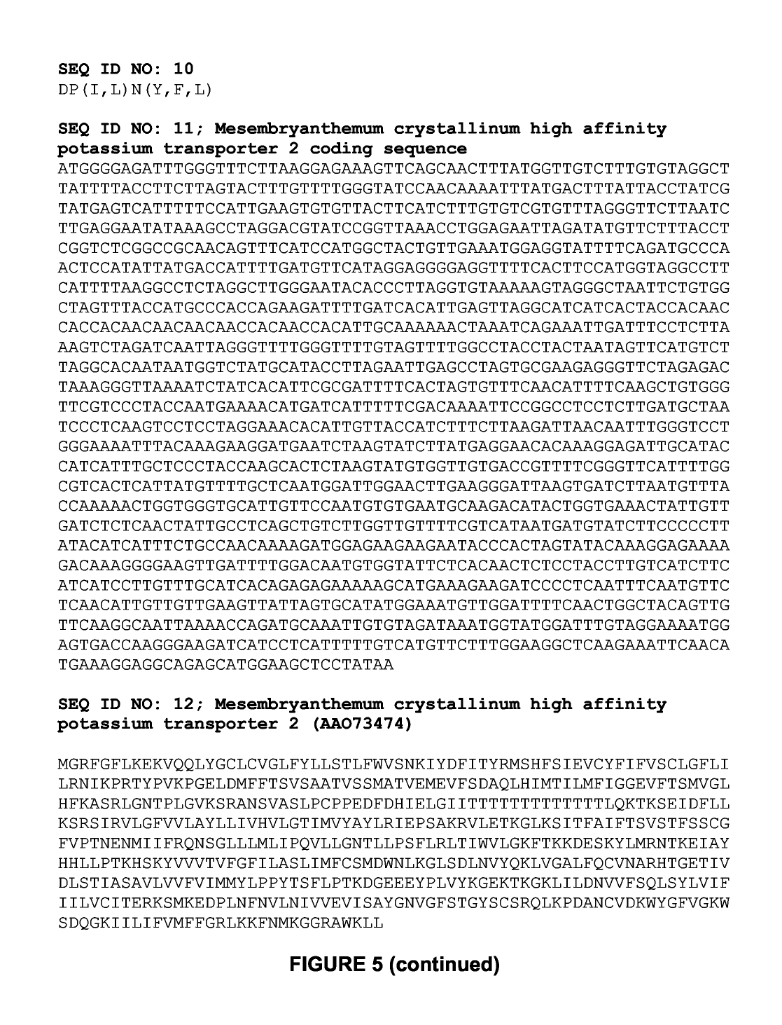

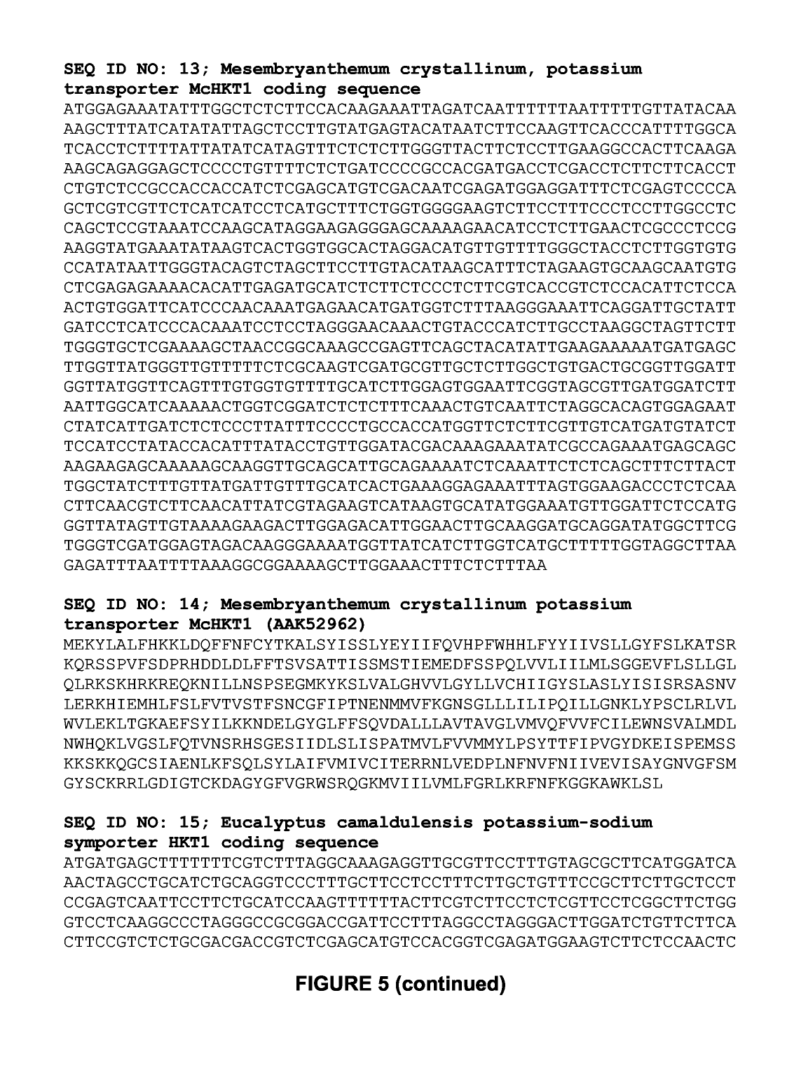

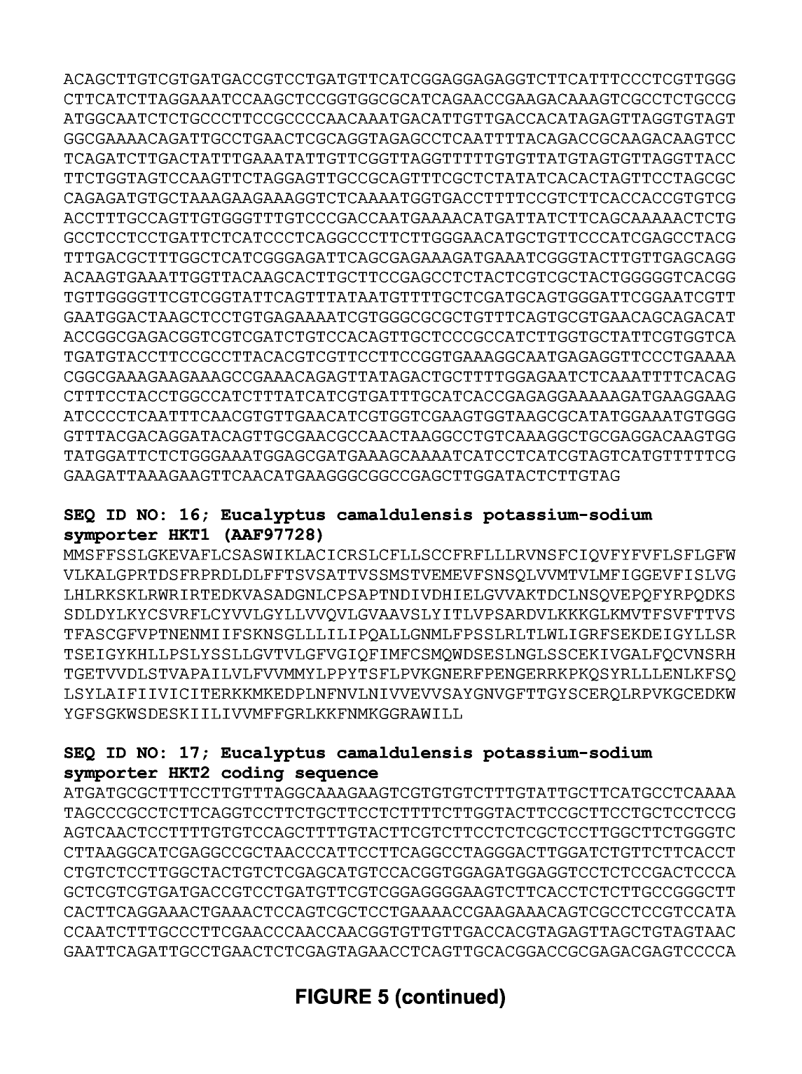

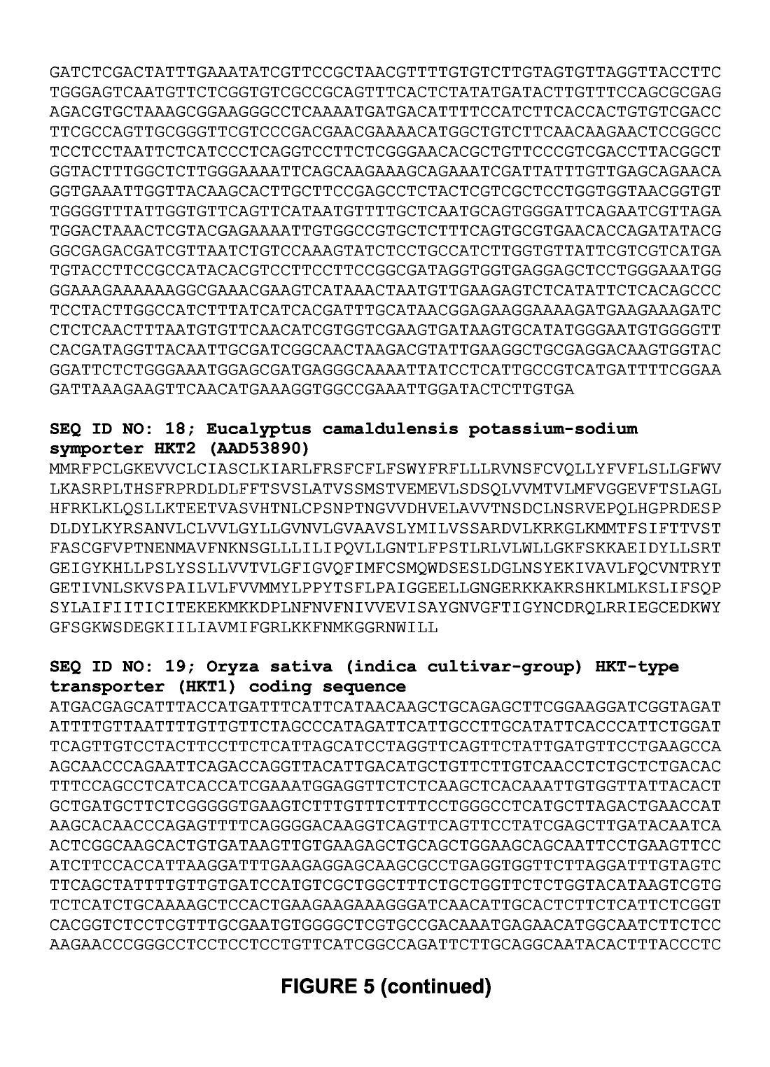

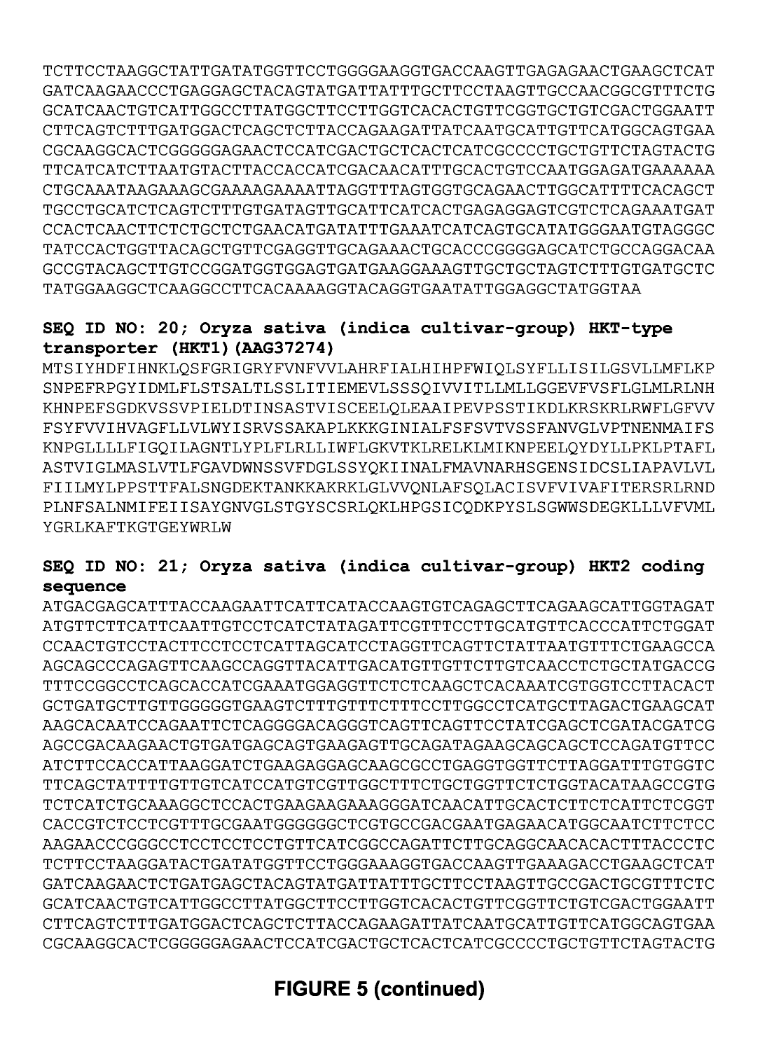

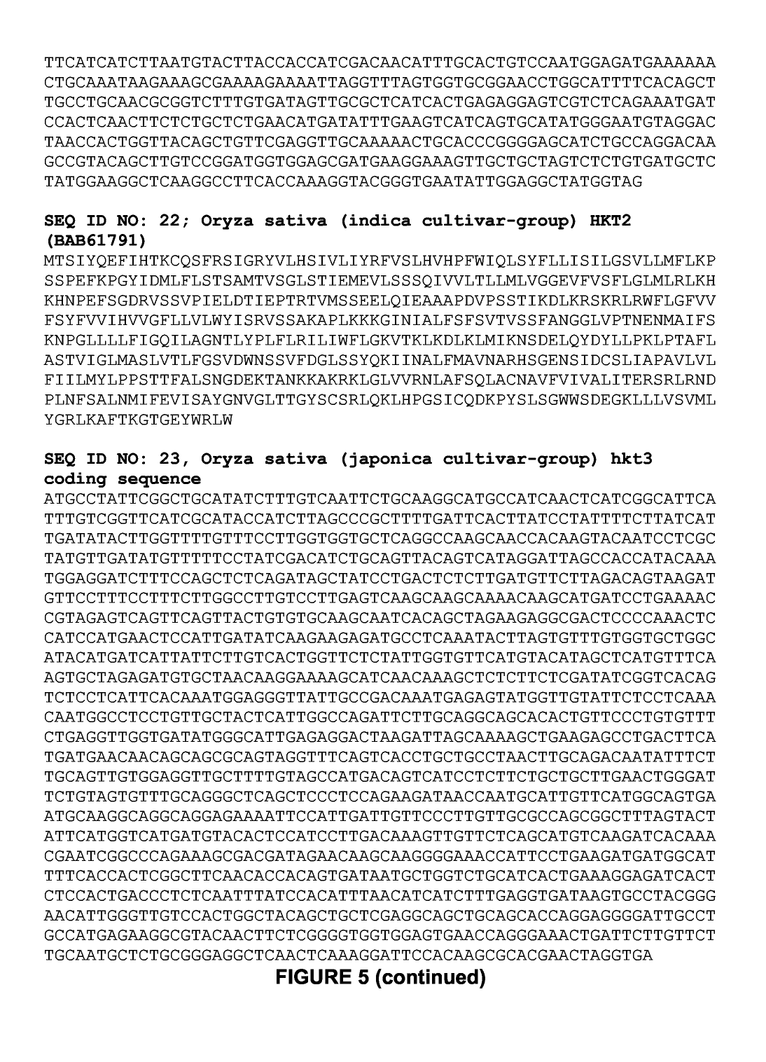

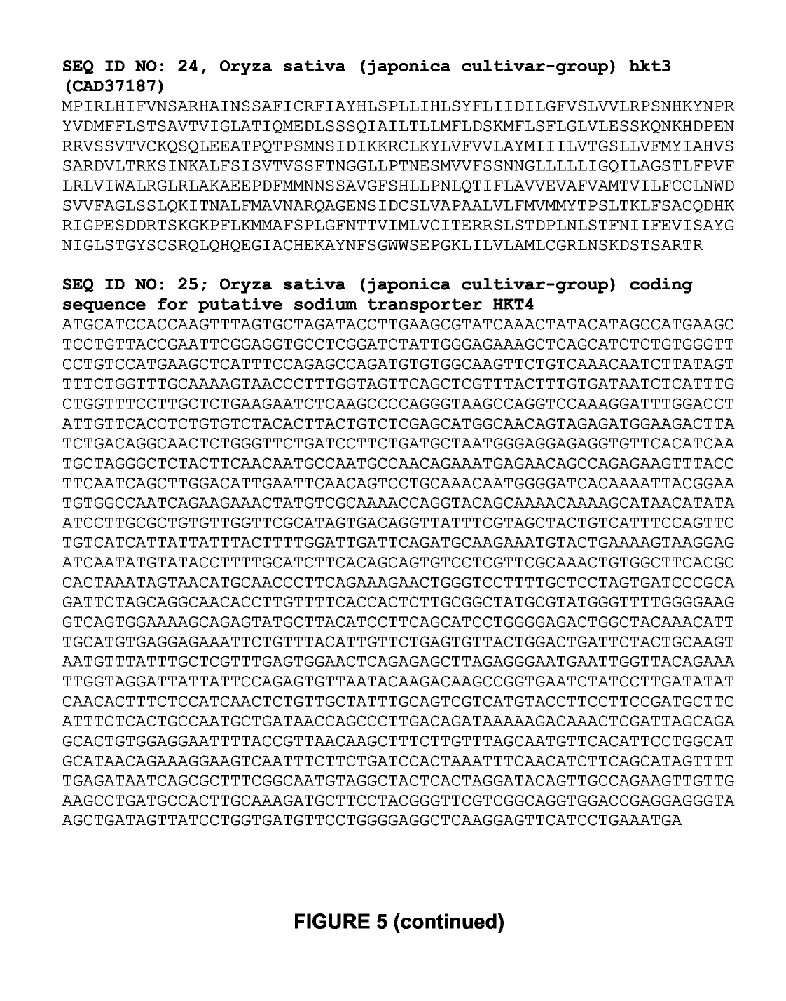

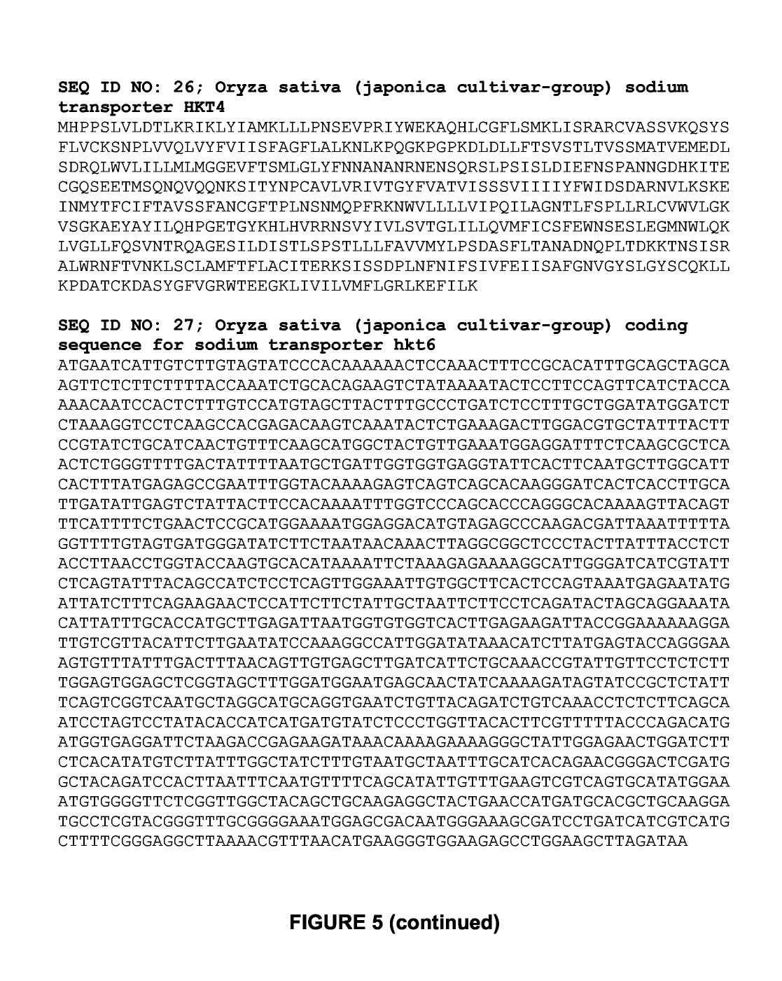

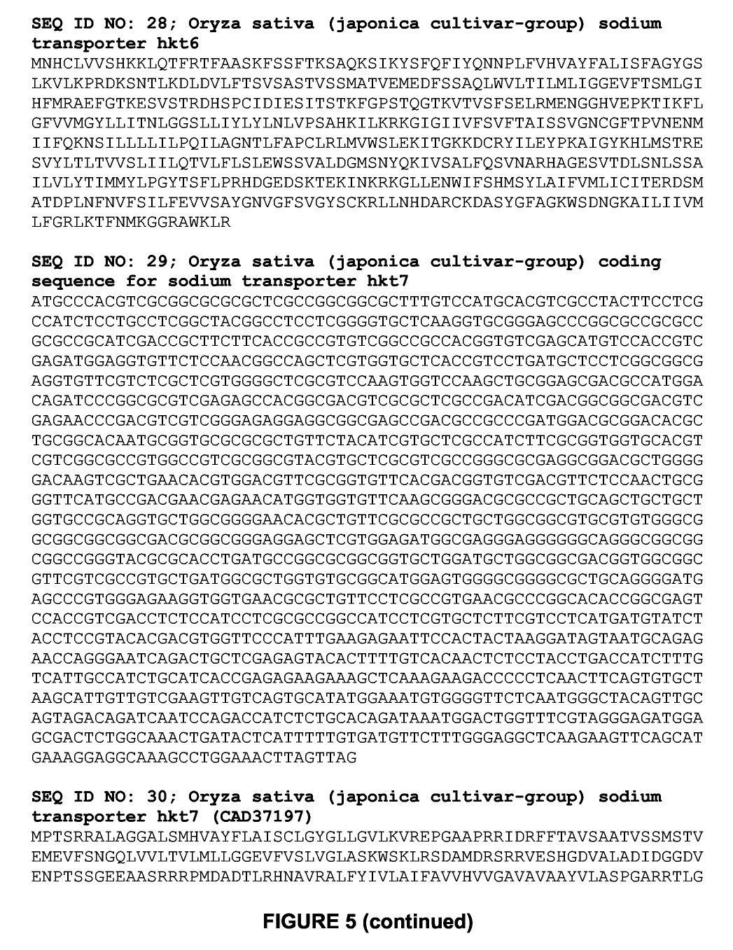

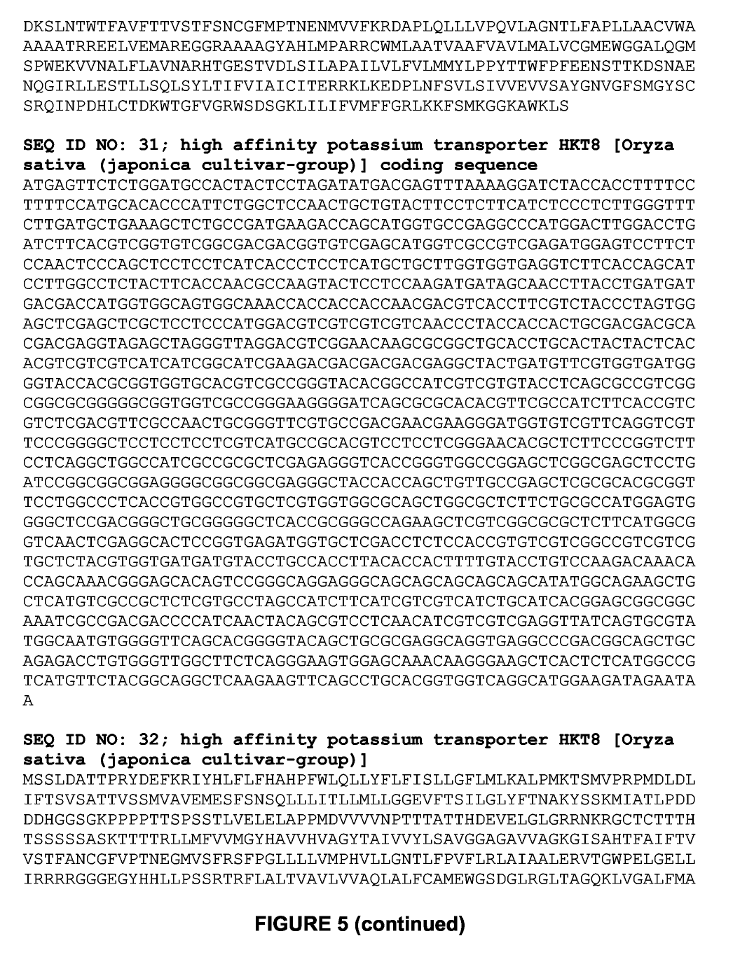

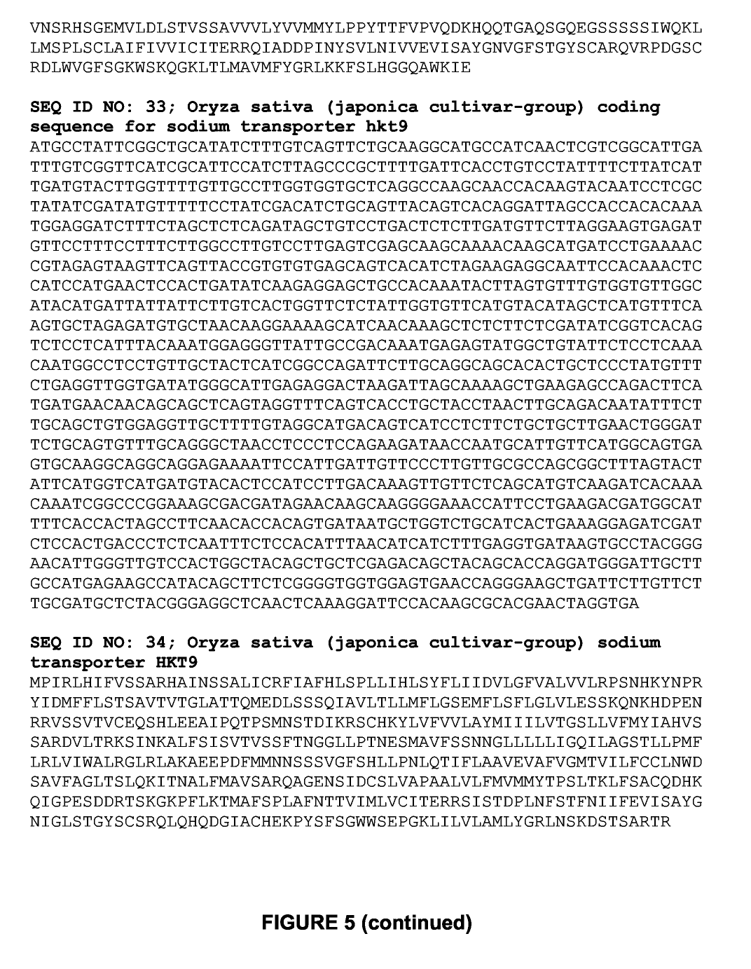

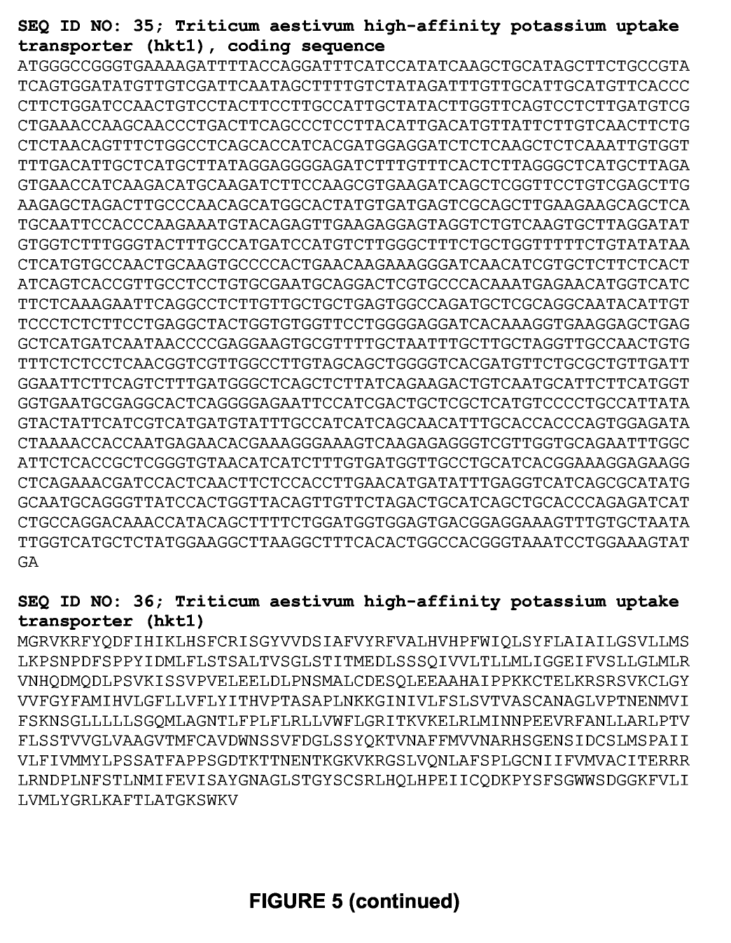

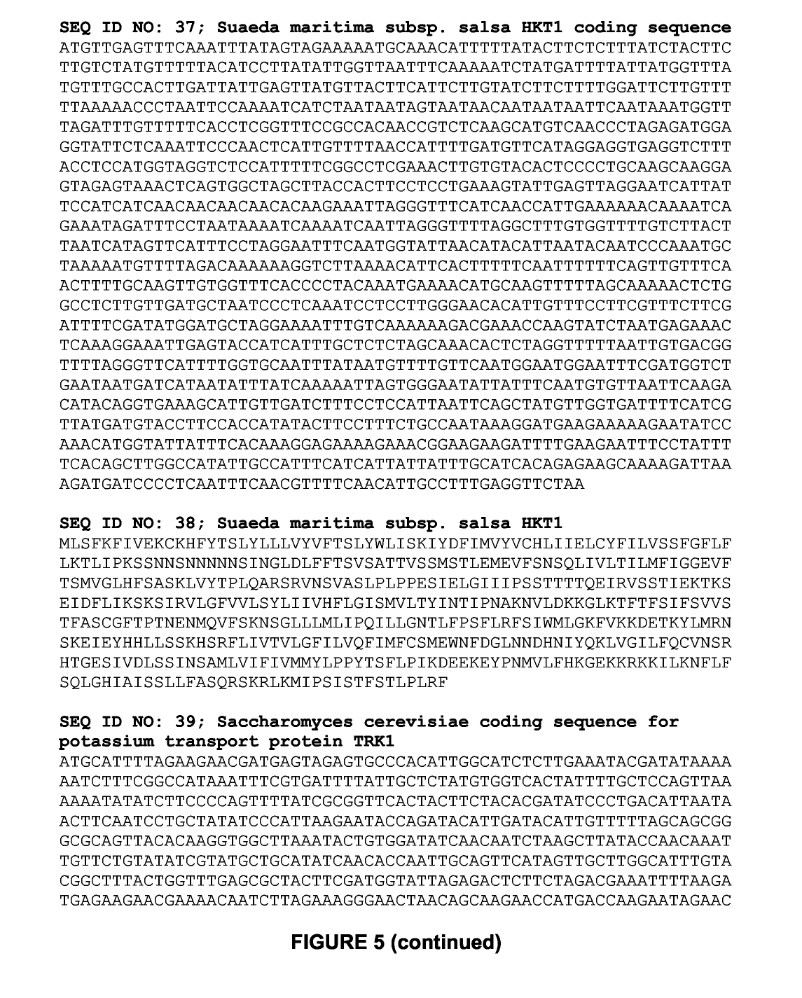

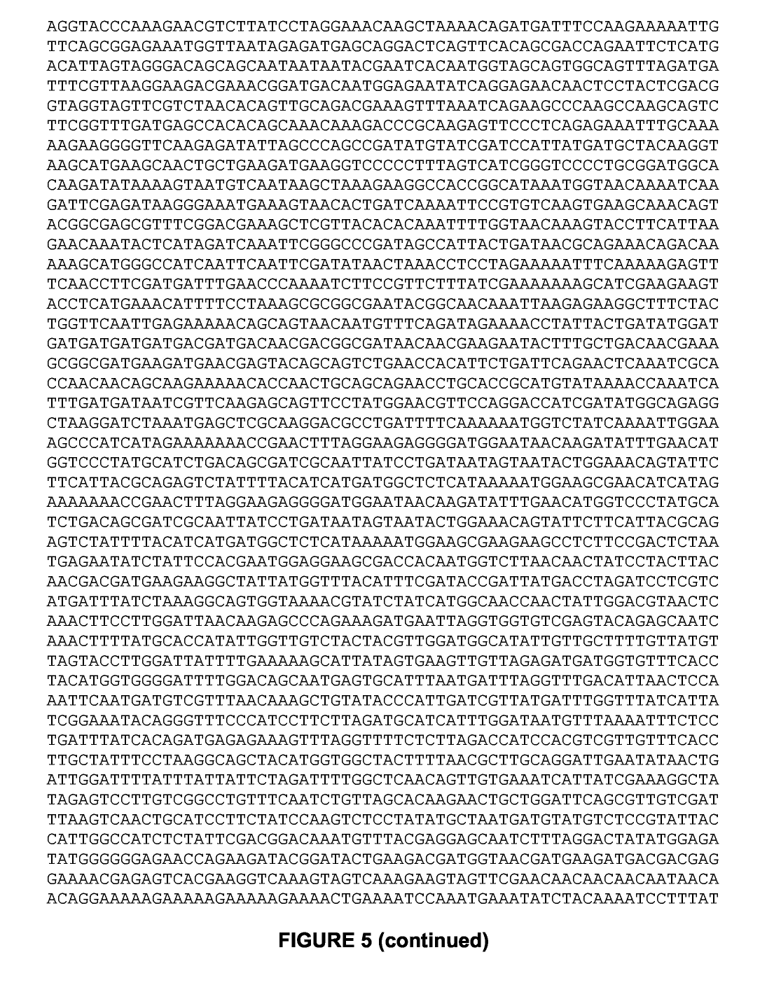

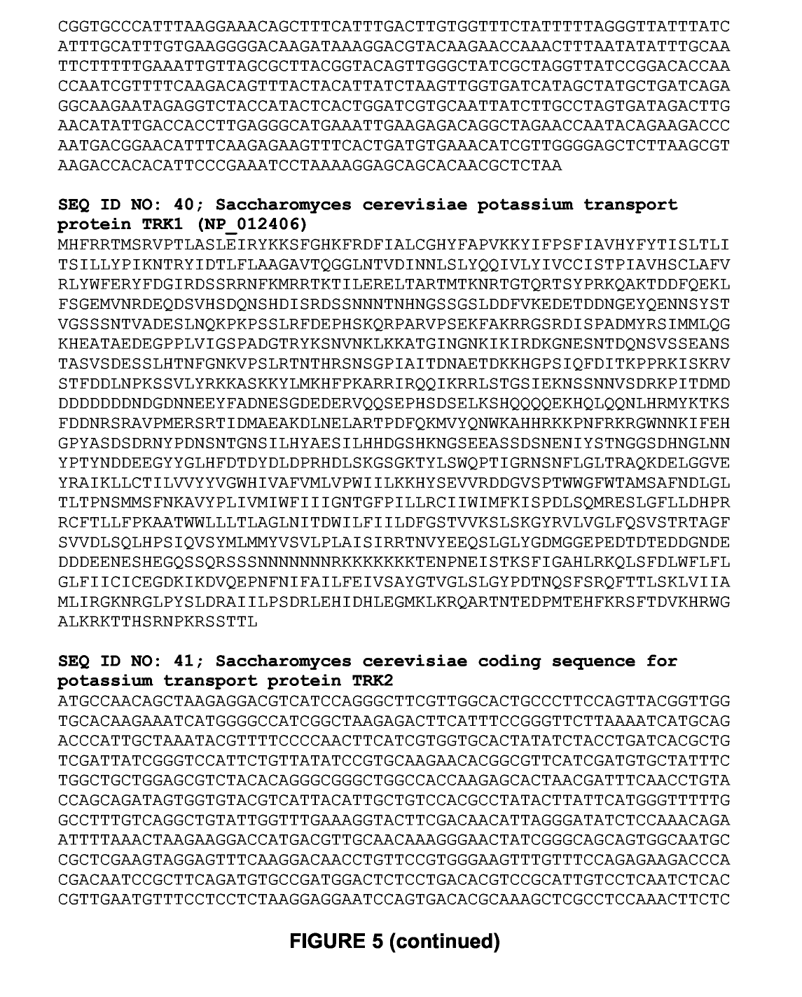

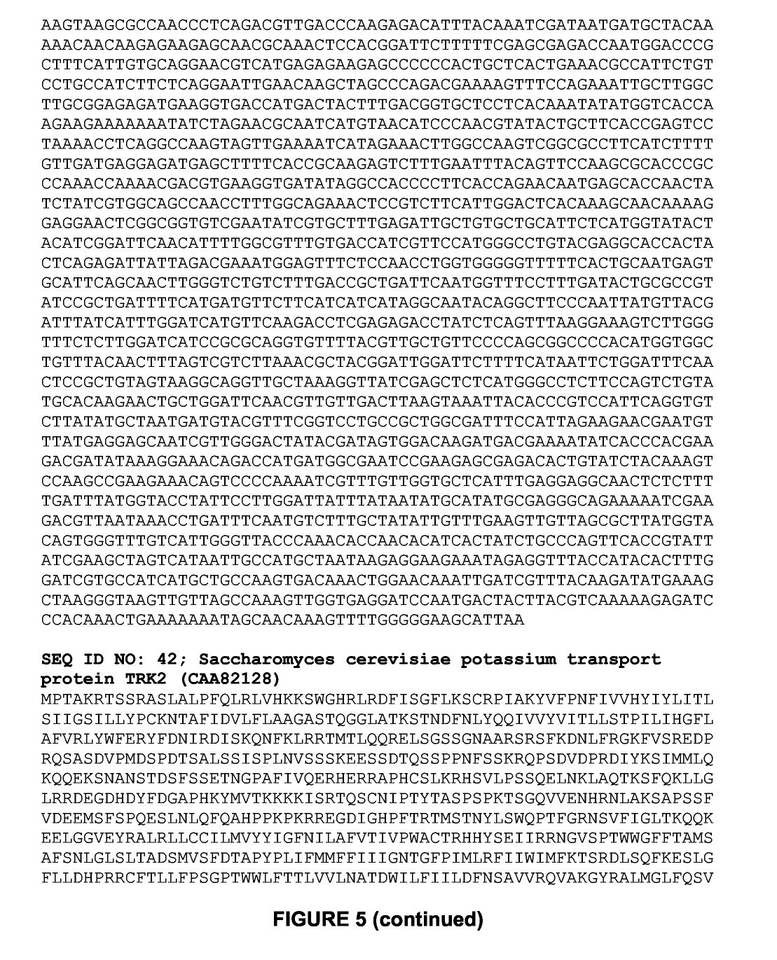

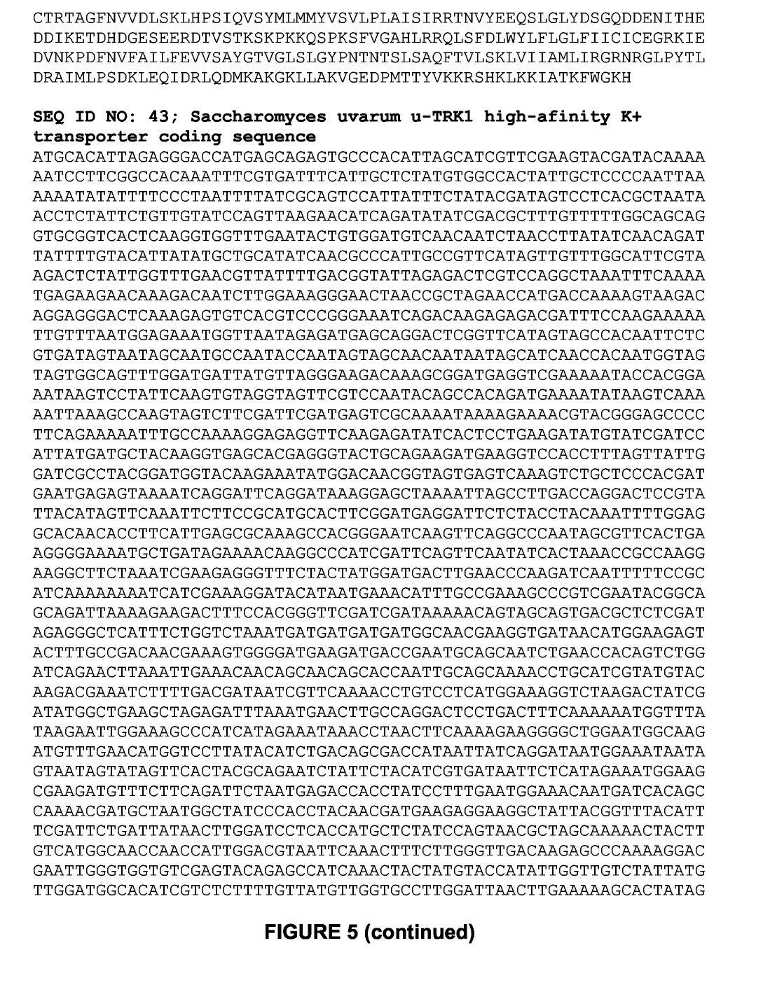

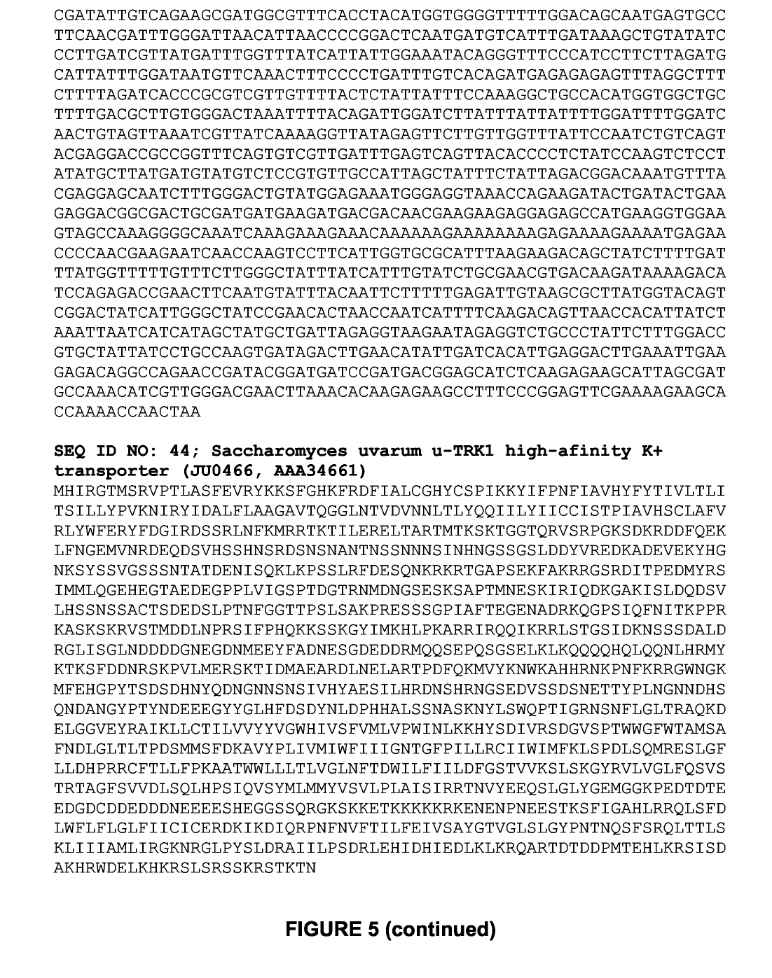

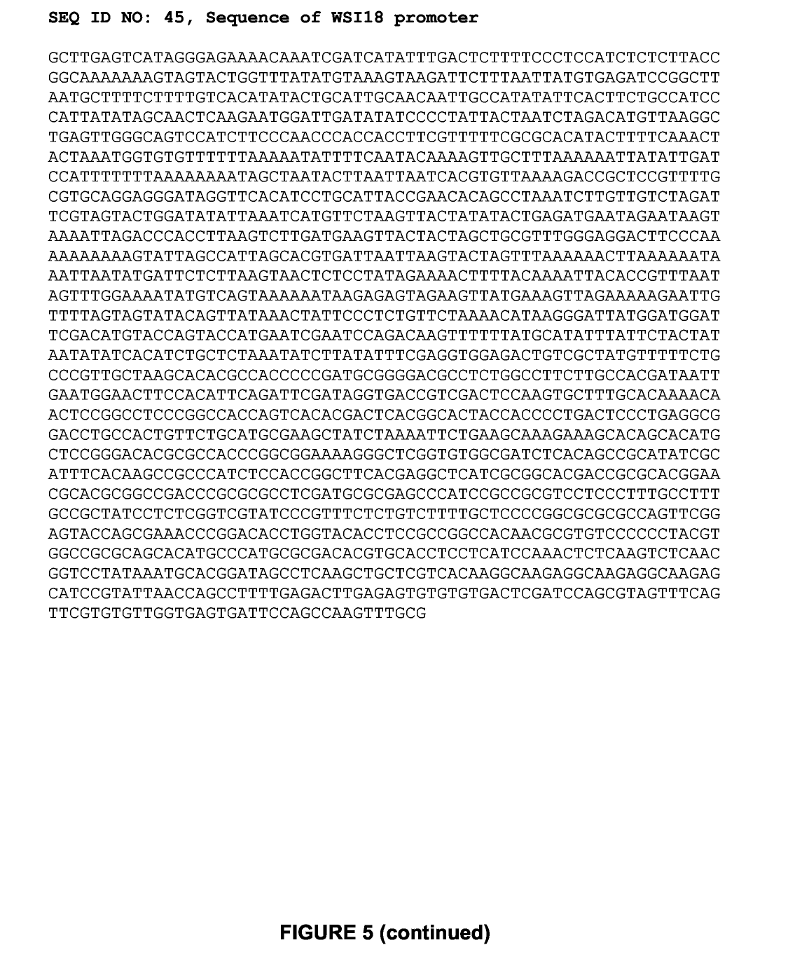

The entry clone p036 was subsequently used in an LR reaction with p56, a destination vector used for Oryza sativa transformation. This vector contains as functional elements within the T-DNA borders: a plant selectable marker; a visual marker; and a Gateway cassette intended for LR in vivo recombination with the sequence of interest already cloned in the entry clone. A rice WSI18 promoter for constitutive expression (PRO0151) was located upstream of this Gateway cassette. After the LR recombination step, the resulting expression vector p060 (FIG. 4) comprising the expression cassette of SEQ ID NO: 4 can be transformed into the Agrobacterium strain LBA4404 and subsequently to Oryza sativa plants. Transformed rice plants were allowed to grow and were then examined for the parameters described in Example 3.

### Example 3

### Evaluation of Transformants

Approximately 15 to 20 independent T0 transformants were generated. The primary transformants were transferred from tissue culture chambers to a greenhouse for growing and harvest of T1 seed. Five events of which the T1 progeny segregated 3:1 for presence/absence of the transgene were retained. For each of these events, 10 T1 seedlings containing the transgene (hetero- and homo-zygotes), and 10 T1 seedlings lacking the transgene (nullizygotes), were selected by visual marker screening. The selected T1 plants were transferred to a greenhouse. Each plant received a unique barcode label to link unambiguously the phenotyping data to the corresponding plant. The selected T1 plants were grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Transgenic plants and the corresponding nullizygotes were grown side-by-side at random positions. From the stage of sowing until the stage of maturity, the plants were passed several times through a digital imaging cabinet. At each time point digital images (2048×1536 pixels, 16 million colours) were taken of each plant from at least 6 different angles.

The mature primary panicles were harvested, bagged, barcode-labelled and then dried for three days in the oven at 37° C. The panicles were then threshed and all the seeds collected. The filled husks were separated from the empty ones using an air-blowing device. After separation, both seed lots were then counted using a commercially available counting machine. The empty husks were discarded. The filled husks were weighed on an analytical balance and the cross-sectional area of the seeds was measured using digital imaging. This procedure resulted in the set of seed-related parameters described below.

These parameters were derived in an automated way from the digital images using image analysis software and were analysed statistically. A two factor ANOVA (analyses of variance) corrected for the unbalanced design was used as statistical model for the overall evaluation of plant phenotypic characteristics. An F-test was carried out on all the parameters measured of all the plants of all the events transformed with that gene. The F-test was carried out to check for an effect of the gene over all the transformation events and to verify for an overall effect of the gene, also named herein “global gene effect”. If the value of the F test shows that the data are significant, than it is concluded that there is a “gene” effect, meaning that not only presence or the position of the gene is causing the effect. The threshold for significance for a true global gene effect is set at 5% probability level for the F test.

To check for an effect of the genes within an event, i.e., for a line-specific effect, a t-test was performed within each event using data sets from the transgenic plants and the corresponding null plants. “Null plants” or “null segregants” or “nullizygotes” are the plants treated in the same way as the transgenic plant, but from which the transgene has segregated. Null plants can also be described as the homozygous negative transformed plants. The threshold for significance for the t-test is set at 10% probability level. The results for some events can be above or below this threshold. This is based on the hypothesis that a gene might only have an effect in certain positions in the genome, and that the occurrence of this position-dependent effect is not uncommon. This kind of gene effect is also named herein a “line effect of the gene”. The p-value is obtained by comparing the t-value to the t-distribution or alternatively, by comparing the F-value to the F-distribution. The p-value then gives the probability that the null hypothesis (i.e., that there is no effect of the transgene) is correct.

The data obtained in the first experiment were confirmed in a second experiment with T2 plants. Three lines that had the correct expression pattern were selected for further analysis. Seed batches from the positive plants (both hetero- and homozygotes) in T1, were screened by monitoring marker expression. For each chosen event, the heterozygote seed batches were then retained for T2 evaluation. Within each seed batch an equal number of positive and negative plants were grown in the greenhouse for evaluation.

A total number of 120 HKT transformed plants were evaluated in the T2 generation, that is 30 plants per event of which 15 positives for the transgene, and 15 negatives.

Because two experiments with overlapping events have been carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment—event—segregants). P-values are obtained by comparing likelihood ratio test to chi square distributions.

### Example 4

### Evaluation of Transformants: Measurement of Yield Related Parameters

Upon analysis of the seeds as described above, the inventors found that plants transformed with the AtHKT gene construct scored better for several yield parameters, including the number of filled seeds, total yield and harvest index when compared to the nullizygous plants. The total seed yield was measured by weighing all filled husks harvested from a plant. The number of filled seeds was determined by counting the number of filled husks that remained after the separation step. The harvest index is defined in the present invention as the ratio between the total seed yield and the above ground area (in mm²).

Four events were selected for an evaluation in T2 plants. The results for increased yield were also present in the T2 generation. The increases for the individual lines varied between 8 to 20% for harvest index, between 12 to 24% for the total weight of seeds and was around 15% for the number of filled seeds.

1. A method for improving growth characteristics of a plant, relative to corresponding wild type plants, comprising increasing activity in a plant of an HKT protein or a homologue thereof and selecting for plants having improved growth characteristics.

2. The method of claim 1, wherein said increased activity is effected by introducing a genetic modification in the locus of a gene encoding an HKT polypeptide or a homologue thereof.

3. The method of claim 2, wherein said genetic modification is effected by one of site-directed mutagenesis, directed evolution, homologous recombination, TILLING or T-DNA activation.

4. A method for improving plant growth characteristics, relative to corresponding wild type plants, comprising introducing and expressing in a plant an HKT nucleic acid molecule or a functional variant thereof.

5. The method of claim 4, wherein said functional variant is a portion of an isolated HKT nucleic acid molecule or a sequence capable of hybridizing to an HKT nucleic acid molecule.

6. The method of claim 4, wherein said isolated HKT nucleic acid molecule or functional variant thereof is overexpressed in a plant.

7. The method of claim 4, wherein said isolated HKT nucleic acid molecule or functional variant thereof is of plant origin.

8. The method of claim 4, wherein said functional variant encodes an orthologue or paralogue of HKT.

9. The method of claim 4, wherein said isolated HKT nucleic acid molecule or functional variant thereof is operably linked to a seed specific promoter.

10. The method of claim 4, wherein said isolated HKT nucleic acid molecule or functional variant thereof is operably linked to an embryo- or aleurone-specific promoter or an embryo- and aleurone-specific promoter.

11. The method of claim 9, wherein said promoter has a comparable expression profile to a WSI18 promoter.

12. The method of claim 1, wherein said improved plant growth characteristic is increased yield relative to corresponding wild type plants.

13. The method of claim 12, wherein said increased yield is increased biomass or increased seed yield or increased biomass and increased seed yield.

14. The method of claim 13, wherein said increased seed yield is selected from one or more of (i) increased seed biomass; (ii) increased number of seeds; (iii) increased number of filled seeds; (iv) increased seed size; (v) increased seed volume; (vi) increased harvest index (HI); or (vii) increased thousand kernel weight (TKW).

15. A plant or plant cell obtained by the method of claim 1.

16. A construct comprising:
(i) an isolated HKT nucleic acid molecule or functional variant thereof;
(ii) one or more seed-specific control sequence(s) capable of driving expression of the nucleic acid sequence of (i); and optionally
(iii) a transcription termination sequence.

17. The construct of claim 16, wherein said seed specific control sequence is an embryo- or aleurone-specific promoter or an embryo- and aleurone-specific promoter.

18. The construct of claim 17, wherein said promoter has a comparable expression profile to a WSI18 promoter.

19. The construct of claim 18, wherein said promoter is as represented in SEQ ID NO: 45.

20. A construct comprising an expression cassette essentially similar to SEQ ID NO 4.

21. A plant or plant cell transformed with the construct of claim 16.

22. A method for the production of a transgenic plant having improved growth characteristics, which method comprises:
(i) introducing into a plant an isolated HKT nucleic acid molecule or functional variant thereof; and
(ii) cultivating the plant cell under conditions for promoting plant growth and development.

23. A transgenic plant or plant cell having improved growth characteristics compared to corresponding wild type plants, which improved growth characteristics result from an isolated HKT nucleic acid molecule or functional variant thereof introduced into said plant or plant cell.

24. The transgenic plant or plant cell of claim 23, wherein said plant is a monocotyledonous plant, or wherein said plant cell is derived from a monocotyledonous plant.

25. Harvestable parts, or products directly derived thereof, of the plant of claim.

26. The harvestable parts, or products directly derived thereof, according to claim 25, wherein said harvestable parts are seeds.

27. (canceled)

28. The method of claim 14, wherein said improved seed yield comprises at least increased number of filled seeds.

29. A method of selecting a plant with improved growth characteristics relative to a corresponding wild type plant, comprising utilizing an isolated HKT nucleic acid molecule or functional variant thereof as a molecular marker.

30. A composition for use as a growth regulator comprising an isolated HKT nucleic acid molecule or functional variant thereof for improving growth and development of plants.

31. A composition for use as a growth regulator comprising an isolated HKT protein or a homologue thereof for improving growth and development of plants.

32. The method of claim 4, wherein said isolated HKT nucleic acid molecule or functional variant thereof is from a dicotyledonous plant.

33. The method of claim 32, wherein said dicotyledonous plant is from the family Brassicaceae.

34. The method of claim 32, wherein said dicotyledonous plant is Arabidopsis thaliana.

35. The transgenic plant or plant cell of claim 24, wherein the monocotyledonous plant is selected from the group consisting of sugar cane, rice, maize, wheat, barley, millet, rye, oats, and sorghum.
